(19) United States
(12) Patent Application Publication
(10) Pub. No.: US 2007/0218061 A1
(43) Pub. Date: Sep. 20, 2007

(54) Use of ephrinb2 directed agents for the treatment or prevention of viral infections

(75) Inventors: Parkash Gill, Agoura Hills, CA (US); Valery Krasnoperov, South Pasadena, CA (US)

Correspondence Address: FISH & NEAVE IP GROUP;ROPES & GRAY LLP, ONE INTERNATIONAL PLACE, BOSTON, MA, 02110-2624, US

(73) Assignee: VasGene Therapeutics, Inc., Broomall, PA (US)

(21) Appl. No.: 11/527,253

(22) Filed: Sep. 25, 2006

Related U.S. Application Data
(60) Provisional application No. 60/719,942, filed on Sep. 23, 2005.

(51) Int. Cl. A61K 48/00; A61K 39/395

(52) U.S. Cl. 424/143.1; 514/12; 514/44

(57) ABSTRACT

In certain embodiments, this present invention provides EphrinB2-targeted agents, including polypeptide compositions and nucleic acid compositions for the treatment or prevention of infections by viruses of the family Paramyxoviridae.

### RELATED APPLICATIONS

This application claims the benefit of priority of U.S. Provisional Application No. 60/719,942 filed Sep. 23, 2005. The entire teachings of the referenced Provisional Application are incorporated herein by reference in its entirety.

### BACKGROUND OF THE INVENTION

Viral pathogens present a significant worldwide health risk and vaccines or therapeutics are often unavailable. Nipah virus (NiV) and the related Hendra virus (HeV) are members of the Henipavirus genus of the Paramyxoviridae. NiV outbreaks have occurred in Malaysia, Singapore and Bangladesh. NiV has a broad host range which includes humans, pigs, dogs, cats, horses, guinea pigs, hamsters, and fruit bats. Therefore, NiV has effects on human health and on agricultural animals and pets. Endothelial cells are the major cellular targets for NiV and HeV, which infect cells through a pH-independent membrane fusion process mediated by their fusion and attachment glycoproteins. Recently, Negrete et al. (Nature 2005 Jul. 21; 436(7049):401-5) and Bonaparte et al. (Proc Natl Acad Sci USA. 2005 Jul. 26; 102(30):10652-7) demonstrated that NiV and HeV use the host protein EphrinB2 as a receptor to gain entry into host cells.

It is an objective of the present disclosure to provide methods and compositions for managing viral infections caused by EphrinB2-binding members of the Paramyxoviridae.

### SUMMARY OF THE INVENTION

In certain aspects, the disclosure provides EphrinB2-targeted agents for the treatment or prevention of Paramyxovirus infections. In certain embodiments, the EphrinB2-targeted agents are polypeptide agents that bind to EphrinB2 or interfere with EphrinB2 mediated functions, including monomeric or dimeric ligand-binding portions of the EphB4 and EphrinB2 proteins and antibodies to EphrinB2. In certain aspects, the EphrinB2-targeted agents are nucleic acid compounds that decrease the expression of EphrinB2. These agents may be used to treat or prevent infections by viruses of the family Paramyxoviridae that bind to EphrinB2, particularly members of the genus Henipavirus.

In certain embodiments, the present disclosure provides methods of inhibiting membrane fusion between a virus of the family Paramyxoviridae (e.g, a Henipavirus) and a target cell (e.g, an endothelial cell) by use of the therapeutic agents relating to EphrinB2 or EphB4.

In certain aspects, the disclosure provides soluble EphB4 polypeptides comprising an amino acid sequence of an extracellular domain of an EphB4 protein. The soluble EphB4 polypeptides bind specifically to an EphrinB2 polypeptide. The term “soluble” is used merely to indicate that these polypeptides do not contain a transmembrane domain or a portion of a transmembrane domain sufficient to compromise the solubility of the polypeptide in a physiological salt solution. Soluble polypeptides are preferably prepared as monomers that compete with EphB4 for binding to ligand such as EphrinB2 and inhibit the signaling that results from EphB4 activation. Optionally, a soluble polypeptide may be prepared in a multimeric form, by, for example, expressing as an Fc fusion protein or fusion with another multimerization domain. Such multimeric forms may have complex activities, having agonistic or antagonistic effects depending on the context. In certain embodiments the soluble EphB4 polypeptide comprises a globular domain of an EphB4 protein. A soluble EphB4 polypeptide may comprise a sequence at least 90% identical to residues 1-522 of the amino acid sequence of SEQ ID NO: 10. A soluble EphB4 polypeptide may comprise a sequence at least 90% identical to residues 1-412 of the amino acid sequence of SEQ ID NO: 10. A soluble EphB4 polypeptide may comprise a sequence at least 90% identical to residues 1-312 of the amino acid sequence of SEQ ID NO: 10. A soluble EphB4 polypeptide may comprise a sequence encompassing the globular (G) domain (amino acids 29-197 of SEQ ID NO: 10), and optionally additional domains, such as the cysteine-rich domain (amino acids 239-321 of SEQ ID NO: 10), the first fibronectin type 3 domain (amino acids 324-429 of SEQ ID NO: 10) and the second fibronectin type 3 domain (amino acids 434-526 of SEQ ID NO: 10). Preferred polypeptides described herein and demonstrated as having ligand binding activity include polypeptides corresponding to 1-537, 1-427 and 1-326, respectively, of the amino acid sequence shown in SEQ ID NO: 10. A soluble EphB4 polypeptide may comprise a sequence as set forth in SEQ ID NO: 1 or 2. As is well known in the art, expression of such EphB4 polypeptides in a suitable cell, such as HEK293T cell line, will result in cleavage of a leader peptide. Although such cleavage is not always complete or perfectly consistent at a single site, it is known that EphB4 tends to be cleaved so as to remove the first 15 amino acids of the sequence shown in SEQ ID NO: 10. Accordingly, as specific examples, the disclosure provides unprocessed soluble EphB4 polypeptides that bind to EphrinB2 and comprise an amino acid sequence selected from the following group (numbering is with respect to the sequence of SEQ ID NO: 10): 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537. Additionally, heterologous leader peptides may be substituted for the endogenous leader sequences. Polypeptides may be used in a processed form, such forms having a predicted amino acid sequence selected from the following group (numbering is with respect to the sequence of SEQ ID NO: 10): 16-197, 16-312, 16-321, 16-326, 16-412, 16-427, 16-429, 16-526 and 16-537. Additionally, a soluble EphB4 polypeptide may be one that comprises an amino acid sequence at least 90%, and optionally 95% or 99% identical to any of the preceding amino acid sequences while retaining EphrinB2 binding activity. Preferably, any variations in the amino acid sequence from the sequence shown in SEQ ID NO: 10 are conservative changes or deletions of no more than 1, 2, 3, 4 or 5 amino acids, particularly in a surface loop region. In certain embodiments, the soluble EphB4 polypeptide may inhibit the interaction between EphrinB2 and EphB4. The soluble EphB4 polypeptide may inhibit clustering of or phosphorylation of EphrinB2 or EphB4. Phosphorylation of EphrinB2 or EphB4 is generally considered to be one of the initial events in triggering intracellular signaling pathways regulated by these proteins. As noted above, the soluble EphB4 polypeptide may be prepared as a monomeric or multimeric fusion protein. The soluble polypeptide may include one or more modified amino acids. Such amino acids may contribute to desirable properties, such as increased resistance to protease digestion.

The present disclosure provides soluble EphB4 polypeptides having an additional component that confers increased serum half-life while still retaining EphrinB2 binding activity. In certain embodiments soluble EphB4 polypeptides are monomeric and are covalently linked to one or more polyoxyaklylene groups (e.g., polyethylene, polypropylene), and preferably polyethylene glycol (PEG) groups. Accordingly, one aspect of the invention provides modified EphB4 polypeptides, wherein the modification comprises a single polyethylene glycol group covalently bonded to the polypeptide. Other aspects provide modified EphB4 polypeptides covalently bonded to one, two, three, or more polyethylene glycol groups.

The one or more PEG may have a molecular weight ranging from about 1 kDa to about 100 kDa, and will preferably have a molecular weight ranging from about 10 to about 60 kDa or about 10 to about 40 kDa. The PEG group may be a linear PEG or a branched PEG. In a preferred embodiment, the soluble, monomeric EphB4 conjugate comprises an EphB4 polypeptide covalently linked to one PEG group of from about 10 to about 40 kDa (monoPEGylated EphB4), or from about 15 to 30 kDa, preferably via an ε-amino group of EphB4 lysine or the N-terminal amino group. Most preferably, EphB4 is randomly PEGylated at one amino group out of the group consisting of the ε-amino groups of EphB4 lysine and the N-terminal amino group.

In one embodiment, the pegylated polypeptides provided by the invention have a serum half-life in vivo at least 50%, 75%, 100%, 150% or 200% greater than that of an unmodified EphB4 polypeptide. In another embodiment, the pegylated EphB4 polypeptides provided by the invention inhibit EphrinB2 activity. In a specific embodiment, they inhibit EphrinB2 receptor clustering, EphrinB2 phosphorylation, and/or EphrinB2 kinase activity.

Surprisingly, it has been found that monoPEGylated EphB4 according to the invention has superior properties in regard to the therapeutic applicability of unmodified soluble EphB4 polypeptides and poly-PEGylated EphB4. Nonetheless, the disclosure also provides poly-PEGylated EphB4 having PEG at more than one position. Such polyPEGylated forms provide improved serum-half life relative to the unmodified form.

In certain embodiments, a soluble EphB4 polypeptide is stably associated with a second stabilizing polypeptide that confers improved half-life without substantially diminishing EphrinB2 binding. A stabilizing polypeptide will preferably be immunocompatible with human patients (or animal patients, where veterinary uses are contemplated) and have little or no significant biological activity.

In a preferred embodiment, the stabilizing polypeptide is a human serum albumin, or a portion thereof. A human serum albumin may be stably associated with the EphB4 polypeptide covalently or non-covalently. Covalent attachment may be achieved by expression of the EphB4 polypeptide as a co-translational fusion with human serum albumin. The albumin sequence may be fused at the N-terminus, the C-terminus or at a non-disruptive internal position in the soluble EphB4 polypeptide. Exposed loops of the EphB4 would be appropriate positions for insertion of an albumin sequence. Albumin may also be post-translationally attached to the EphB4 polypeptide by, for example, chemical cross-linking. An EphB4 polypeptide may also be stably associated with more than one albumin polypeptide. In some embodiments, the albumin is selected from the group consisting of a human serum albumin (HSA) and bovine serum albumin (BSA). In other embodiments, the albumin is a naturally occurring variant. In one preferred embodiment, the EphB4-HSA fusion inhibits the interaction between EphrinB2 and EphB4, the clustering of EphrinB2 or EphB4, the phosphorylation of EphrinB2 or EphB4, or combinations thereof. In other embodiments, the EphB4-HSA fusion has enhanced in vivo stability relative to the unmodified wildtype polypeptide.

In certain aspects, the disclosure provides soluble EphrinB2 polypeptides comprising an amino acid sequence of an extracellular domain of an EphrinB2 protein. The soluble EphrinB2 polypeptides bind specifically to an EphB4 polypeptide. The term “soluble” is used merely to indicate that these polypeptides do not contain a transmembrane domain or a portion of a transmembrane domain sufficient to compromise the solubility of the polypeptide in a physiological salt solution. Soluble polypeptides are preferably prepared as monomers that compete with EphrinB2 for binding to ligand such as EphB4 and inhibit the signaling that results from EphrinB2 activation. Optionally, a soluble polypeptide may be prepared in a multimeric form, by, for example, expressing as an Fc fusion protein or fusion with another multimerization domain. Such multimeric forms may have complex activities, having agonistic or antagonistic effects depending on the context. A soluble EphrinB2 polypeptide may comprise residues 1-225 of the amino acid sequence defined by SEQ ID NO: 11. A soluble EphrinB2 polypeptide may comprise a sequence defined by SEQ ID NO: 3. As is well known in the art, expression of such EphrinB2 polypeptides in a suitable cell, such as HEK293T cell line, will result in cleavage of a leader peptide. Although such cleavage is not always complete or perfectly consistent at a single site, it is known that EphrinB2 tends to be cleaved so as to remove the first 26 amino acids of the sequence shown in SEQ ID NO: 11. Accordingly, as specific examples, the disclosure provides unprocessed soluble EphrinB2 polypeptides that bind to EphB4 and comprise an amino acid sequence corresponding to amino acids 1-225 of SEQ ID NO: 11. Such polypeptides may be used in a processed form, such forms having a predicted amino acid sequence selected from the following group (numbering is with respect to the sequence of SEQ ID NO: 11): 26-225. In certain embodiments, the soluble EphrinB2 polypeptide may inhibit the interaction between EphrinB2 and EphB4. The soluble EphrinB2 polypeptide may inhibit clustering of or phosphorylation of EphrinB2 or EphB4. As noted above, the soluble EphrinB2 polypeptide may be prepared as a monomeric or multimeric fusion protein. The soluble polypeptide may include one or more modified amino acids. Such amino acids may contribute to desirable properties, such as increased resistance to protease digestion.

In certain aspects, the disclosure provides isolated nucleic acid compounds comprising at least a portion that hybridizes to an EphrinB2 transcript under physiological conditions and decreases the expression of EphrinB2 in a cell. The EphrinB2 transcript may be any pre-splicing transcript (i.e., including introns), post-splicing transcript, as well as any splice variant. In certain embodiments, the EphrinB2 transcript has a sequence set forth in SEQ ID NO: 9. Examples of categories of nucleic acid compounds include antisense nucleic acids, RNAi constructs and catalytic nucleic acid constructs. A nucleic acid compound may be single or double stranded. A double stranded compound may also include regions of overhang or non-complementarity, where one or the other of the strands is single stranded. A single stranded compound may include regions of self-complementarity, meaning that the compound forms a so-called “hairpin” or “stem-loop” structure, with a region of double helical structure. A nucleic acid compound may comprise a nucleotide sequence that is complementary to a region consisting of no more than 1000, no more than 500, no more than 250, no more than 100 or no more than 50 nucleotides of the EphrinB2 nucleic acid sequence as designated by SEQ ID NO: 9. The region of complementarity will preferably be at least 8 nucleotides, and optionally at least 10 or at least 15 nucleotides. A region of complementarity may fall within an intron, a coding sequence or a noncoding sequence of an EphrinB2 transcript, such as the coding sequence portion of the sequences set forth in SEQ ID NO: 9. Generally, a nucleic acid compound will have a length of about 8 to about 500 nucleotides or base pairs in length, and optionally the length will be about 14 to about 50 nucleotides. A nucleic acid may be a DNA (particularly for use as an antisense), RNA or RNA:DNA hybrid. Any one strand may include a mixture of DNA and RNA, as well as modified forms that cannot readily be classified as either DNA or RNA. Likewise, a double stranded compound may be DNA:DNA, DNA:RNA or RNA:RNA, and any one strand may also include a mixture of DNA and RNA, as well as modified forms that cannot readily be classified as either DNA or RNA. A nucleic acid compound may include any of a variety of modifications, including one or modifications to the backbone (the sugar-phosphate portion in a natural nucleic acid, including internucleotide linkages) or the base portion (the purine or pyrimidine portion of a natural nucleic acid). An antisense nucleic acid compound will preferably have a length of about 15 to about 30 nucleotides and will often contain one or more modifications to improve characteristics such as stability in the serum, in a cell or in a place where the compound is likely to be delivered, such as the stomach in the case of orally delivered compounds and the lung for inhaled compounds. Examples of various EphrinB2 antisense and RNAi constructs having differing levels of efficacy are presented in Tables 1-2. In the case of an RNAi construct, the strand complementary to the target transcript will generally be RNA or modifications thereof. The other strand may be RNA, DNA or any other variation. The duplex portion of double stranded or single stranded “hairpin” RNAi construct will preferably have a length of 18 to 25 nucleotides in length and optionally about 21 to 23 nucleotides in length. Catalytic or enzymatic nucleic acids may be ribozymes or DNA enzymes and may also contain modified forms. Nucleic acid compounds may inhibit expression of the target by about 50%, 75%, 90% or more when contacted with cells under physiological conditions and at a concentration where a nonsense or sense control has little or no effect. Preferred concentrations for testing the effect of nucleic acid compounds are 1, 5 and 10 micromolar. Nucleic acid compounds may also be tested for effects on cellular phenotypes. In the case of certain cancer cell lines, cell death or decreased rate of expansion may be measured upon administration of EphB4 or EphrinB2-targeted nucleic acid compounds. Preferably, cell expansion will be inhibited by greater than 50% at an experimentally meaningful concentration of the nucleic acid.

In certain aspects, the disclosure provides pharmaceutical or vaccine formulations comprising an EphrinB2-targeted agent disclosed herein reagent and a pharmaceutically acceptable carrier. The disclosure further provides the use of EphrinB2-targeted agents for the preparation of a medicament or vaccine for the treatment or prevention of infections by members of the Paramyxoviridae, particularly members of the genus Henipavirus and preferably those viruses that bind to EphrinB2.

### BRIEF DESCRIPTION OF THE DRAWINGS

FIG. 1 shows amino acid sequence of the B4ECv3 protein (predicted sequence of the precursor including uncleaved Eph B4 leader peptide is shown; SEQ ID NO: 1).

FIG. 2 shows amino acid sequence of the B4ECv3NT protein (predicted sequence of the precursor including uncleaved Eph B4 leader peptide is shown; SEQ ID NO: 2).

FIG. 3 shows amino acid sequence of the B2EC protein (predicted sequence of the precursor including uncleaved EphrinB2 leader peptide is shown; SEQ ID NO: 3).

FIG. 4 shows amino acid sequence of the B4ECv3-FC protein (predicted sequence of the precursor including uncleaved Eph B4 leader peptide is shown; SEQ ID NO: 4).

FIG. 5 shows amino acid sequence of the B2EC-FC protein (predicted sequence of the precursor including uncleaved EphrinB2 leader peptide is shown; SEQ ID NO: 5).

FIG. 6 shows B4EC-FC binding assay (Protein A-agarose based).

FIG. 7 shows B4EC-FC inhibition assay (Inhibition in solution).

FIG. 8 shows B2EC-FC binding assay (Protein-A-agarose based assay).

FIG. 9 is a schematic representation of human EphrinB2 constructs.

FIG. 10 is a schematic representation of human EphB4 constructs.

FIG. 11 shows the domain structure of the recombinant soluble EphB4EC proteins. Designation of the domains are as follows: L—leader peptide, G—globular (ligand-binding domain), C—Cys-rich domain, F1, F2—fibronectin type III repeats, H—6×His-tag.

FIG. 12 shows purification and ligand binding properties of the EphB4EC proteins. A. SDS-PAAG gel electrophoresis of purified EphB4-derived recombinant soluble proteins (Coomassie-stained). B. Binding of EphrinB2-AP fusion to EphB4-derived recombinant proteins immobilized on Ni-NTA-agarose beads. Results of three independent experiments are shown for each protein. Vertical axis—optical density at 420 nm.

FIG. 13 shows tyrosine phosphorylation of EphB4 receptor in PC3 cells in response to stimulation with EphrinB2-Fc fusion in presence or absence of EphB4-derived recombinant soluble proteins.

FIG. 14 shows four human EphB4 constructs.

FIG. 15 shows three human EphrinB2 constructs.

FIGS. 16A-B show a cDNA nucleotide sequence of human EphB4 (SEQ ID NO: 8).

FIGS. 17A-B show a cDNA nucleotide sequence of human EphrinB2 (SEQ ID NO: 9).

FIG. 18 shows an amino acid sequence of human EphB4 (SEQ ID NO: 10).

FIG. 19 shows an amino acid sequence of human EphrinB2 (SEQ ID NO: 11).

FIG. 20 shows a comparison of the EphrinB2 binding properties of the HSA-EphB4 fusion protein and other EphB4 polypeptides.

FIG. 21 shows a comparison between the in vivo stability of an EphB4-HSA fusion protein and an EphB4 polypeptide in mice.

FIG. 22 shows the EphrinB2 binding activity of soluble EphB4 polypeptides pegylated under specific pH conditions.

FIG. 23 shows the chromatographic separation of PEG derivatives of EphB4 protein on SP-Sepharose columns. Purity of the PEG-modified EphB4 protein was analyzed by PAGE. The EphrinB2 binding of the pegylation reaction products is also shown.

FIG. 24 shows the purity, as determined by SDS-PAGE, of chromatography-separated unpegylated, monopegylated and poly-pegylated EphB4 fractions.

FIG. 25 shows the EphrinB2-binding activity of the chromatography fractions from the EphB4 pegylation reaction.

FIG. 26 shows the retention of EphrinB2-binding activity of the chromatography fractions from the EphB4 pegylation reaction after incubation in mouse serum at 37° C. for three days.

FIG. 27 shows the in vivo stability of unpegylated, monopegylated and polypegylated EphB4 in mice over time.

### DETAILED DESCRIPTION OF THE INVENTION

I. Overview

Recently, Negrete et al. (Nature 2005 Jul. 21; 436(7049):401-5) and Bonaparte et al. (Proc Natl Acad Sci USA. 2005 Jul. 26; 102(30):10652-7) demonstrated that NiV and HeV use the host protein EphrinB2 as a receptor to gain entry into host cells. The current invention is based in part on the insight that certain EphrinB2-targeted agents can be used to treat or prevent infections by members of the Paramyxoviridae, and particularly those that use EphrinB2 as a receptor for entry into host cells, such as members of the genus Henipnavirus, e.g., HeV and NiV. In certain embodiments, the EphrinB2-targeted agents disclosed are polypeptides, including soluble monomeric polypeptides of the extracellular domains of EphrinB2 or EphB4 and antibodies that bind to portions of EphrinB2. Applicants have generated modified forms of EphrinB2 and EphB4 polypeptides and have demonstrated that such modified forms have markedly improved pharmacokinetic properties. In certain embodiments, the EphrinB2-targeted agents disclosed are antisense and siRNA nucleic acids that inhibit EphrinB2 expression.

As used herein, the terms Ephrin and Eph are used to refer, respectively, to ligands and receptors. They can be from any of a variety of animals (e.g., mammals/non-mammals, vertebrates/non-vertebrates, including humans). The nomenclature in this area has changed rapidly and the terminology used herein is that proposed as a result of work by the Eph Nomenclature Committee, which can be accessed, along with previously-used names at web site http://www.eph-nomenclature.com.

The work described herein, particularly in the examples, refers to EphrinB2 and EphB4. However, the present invention contemplates any ephrin ligand and/or Eph receptor within their respective family, which is expressed in a tumor. The ephrins (ligands) are of two structural types, which can be further subdivided on the basis of sequence relationships and, functionally, on the basis of the preferential binding they exhibit for two corresponding receptor subgroups. Structurally, there are two types of ephrins: those which are membrane-anchored by a glycerophosphatidylinositol (GPI) linkage and those anchored through a transmembrane domain. Conventionally, the ligands are divided into the Ephrin-A subclass, which are GPI-linked proteins which bind preferentially to EphA receptors, and the Ephrin-B subclass, which are transmembrane proteins which generally bind preferentially to EphB receptors.

The Eph family receptors are a family of receptor protein-tyrosine kinases which are related to Eph, a receptor named for its expression in an erythropoietin-producing human hepatocellular carcinoma cell line. They are divided into two subgroups on the basis of the relatedness of their extracellular domain sequences and their ability to bind preferentially to Ephrin-A proteins or Ephrin-B proteins. Receptors which interact preferentially with Ephrin-A proteins are EphA receptors and those which interact preferentially with Ephrin-B proteins are EphB receptors.

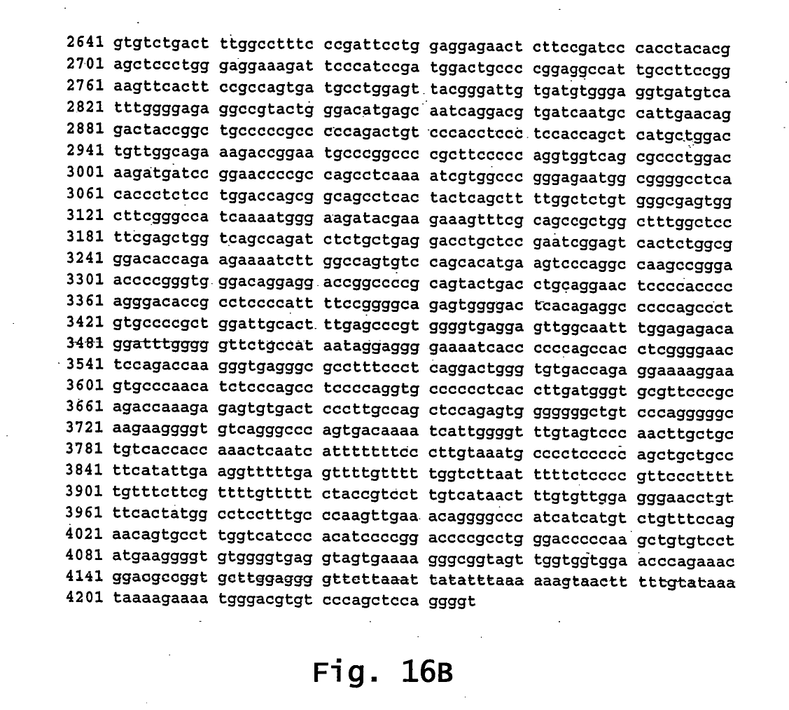

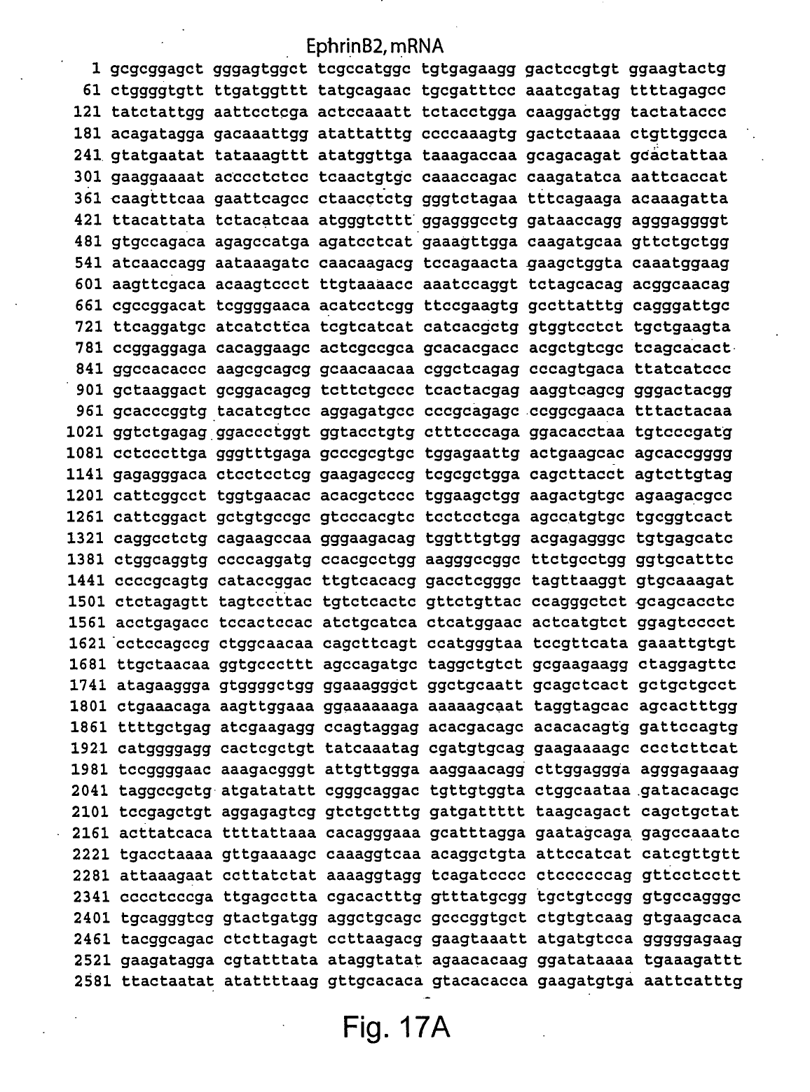

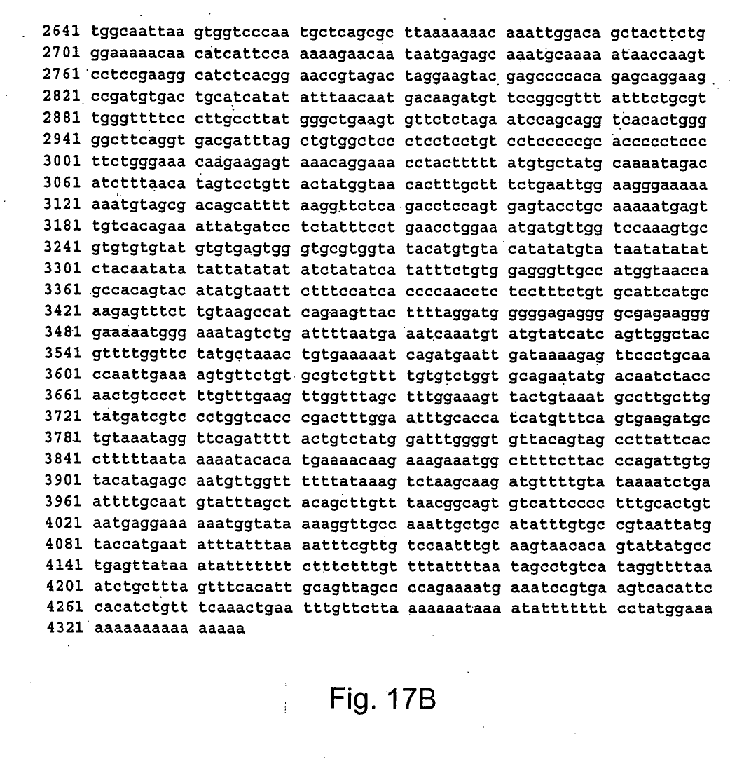

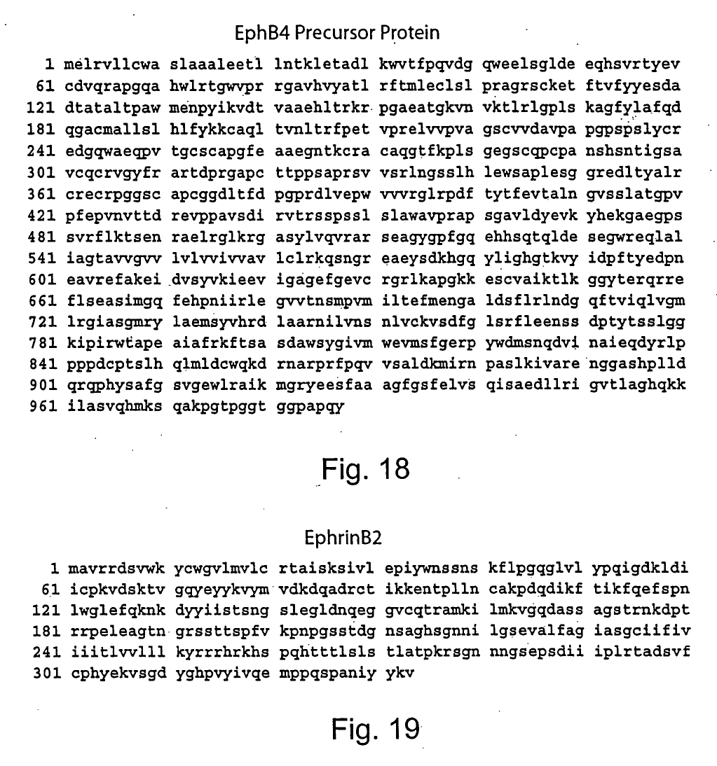

Eph receptors have an extracellular domain composed of the ligand-binding globular domain, a cysteine rich region followed by a pair of fibronectin type III repeats (e.g., see FIG. 16). The cytoplasmic domain consists of a juxtamembrane region containing two conserved tyrosine residues; a protein tyrosine kinase domain; a sterile α-motif (SAM) and a PDZ-domain binding motif. EphB4 is specific for the membrane-bound ligand EphrinB2 (Sakano, S. et al 1996; Brambilla R. et al 1995). EphrinB2 belongs to the class of Eph ligands that have a transmembrane domain and cytoplasmic region with five conserved tyrosine residues and PDZ domain. Eph receptors are activated by binding of clustered, membrane attached ephrins (Davis S et al, 1994), indicating that contact between cells expressing the receptors and cells expressing the ligands is required for Eph activation.

Upon ligand binding, an Eph receptor dimerizes and autophosphorylates the juxtamembrane tyrosine residues to acquire full activation (Kalo M S et al, 1999, Binns K S, 2000). In addition to forward signaling through the Eph receptor, reverse signaling can occur through the ephrin Bs. Eph engagement of ephrins results in rapid phosphorylation of the conserved intracellular tyrosines (Bruckner K, 1997) and somewhat slower recruitment of PDZ binding proteins (Palmer A 2002). Recently, several studies have shown that high expression of Eph/ephrins may be associated with increased potentials for tumor growth, tumorigenicity, and metastasis (Easty D J, 1999; Kiyokawa E, 1994; Tang X X, 1999; Vogt T, 1998; Liu W, 2002; Stephenson S A, 2001; Steube K G 1999; Berclaz G, 1996).

In certain embodiments, the present invention provides polypeptide therapeutic agents that inhibit activity of EphrinB2, EphB4, or both. As used herein, the term “polypeptide therapeutic agent” or “polypeptide agent” is a generic term which includes any polypeptide that blocks signaling through the EphrinB2/EphB4 pathway. A preferred polypeptide therapeutic agent of the invention is a soluble polypeptide of EphrinB2 or EphB4. Another preferred polypeptide therapeutic agent of the invention is an antagonist antibody that binds to EphrinB2 or EphB4. For example, such polypeptide therapeutic agent can inhibit function of EphrinB2 or EphB4, inhibit the interaction between EphrinB2 and EphB4, inhibit the phosphorylation of EphrinB2 or EphB4, or inhibit any of the downstream signaling events upon binding of EphrinB2 to EphB4. Such polypeptides may include EphB4 or EphrinB2 that are modified so as to improve serum half-life, such as by PEGylation or stable association with a serum albumin protein.

II. Soluble Polypeptides

In certain aspects, the invention relates to a soluble polypeptide comprising an extracellular domain of an EphrinB2 protein (referred to herein as an EphrinB2 soluble polypeptide) or comprising an extracellular domain of an EphB4 protein (referred to herein as an EphB4 soluble polypeptide). Preferably, the subject soluble polypeptide is a monomer and is capable of binding with high affinity to EphrinB2 or EphB4. In a specific embodiment, the EphB4 soluble polypeptide of the invention comprises a globular domain of an EphB4 protein. Specific examples EphB4 soluble polypeptides are provided in SEQ ID NOs: 1, 2, 12, 13, 14, 16, 17, 18, 19, and 20. Specific examples of EphrinB2 soluble polypeptides are provided in SEQ ID NOs: 3 and 5.

As used herein, the subject soluble polypeptides include fragments, functional variants, and modified forms of EphB4 soluble polypeptide or an EphrinB2 soluble polypeptide. These fragments, functional variants, and modified forms of the subject soluble polypeptides antagonize function of EphB4, EphrinB2 or both.

In certain embodiments, isolated fragments of the subject soluble polypeptides can be obtained by screening polypeptides recombinantly produced from the corresponding fragment of the nucleic acid encoding an EphB4 or EphrinB2 soluble polypeptides. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function to inhibit function of EphB4 or EphrinB2, for example, by testing the ability of the fragments to bind to EphrinB2 or EphB4, inhibit EphrinB2 or EphB4 kinase activity, or inhibit viral binding to cell or viral infection of a cell.

In certain embodiments, a functional variant of an EphB4 soluble polypeptide comprises an amino acid sequence that is at least 90%, 95%, 97%, 99% or 100% identical to residues 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of the amino acid sequence defined by SEQ ID NO: 10. Such polypeptides may be used in a processed form, and accordingly, in certain embodiments, an EphB4 soluble polypeptide comprises an amino acid sequence that is at least 90%, 95%, 97%, 99% or 100% identical to residues 16-197, 16-312, 16-321, 16-326, 16-412, 16-427, 16-429, 16-526 and 16-537 of the amino acid sequence defined by SEQ ID NO: 10.

In other embodiments, a functional variant of an EphrinB2 soluble polypeptide comprises a sequence at least 90%, 95%, 97%, 99% or 100% identical to residues 1-225 of the amino acid sequence defined by SEQ ID NO: 11 or a processed form, such as one comprising a sequence at least 90%, 95%, 97%, 99% or 100% identical to residues 26-225 of the amino acid sequence defined by SEQ ID NO: 11.

In certain embodiments, the present invention contemplates making functional variants by modifying the structure of the subject soluble polypeptide for such purposes as enhancing therapeutic or prophylactic efficacy, or stability (e.g., ex vivo shelf life and resistance to proteolytic degradation in vivo). Such modified soluble polypeptide are considered functional equivalents of the naturally-occurring EphB4 or EphrinB2 soluble polypeptide. Modified soluble polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

This invention further contemplates a method of generating sets of combinatorial mutants of the EphB4 or EphrinB2 soluble polypeptides, as well as truncation mutants, and is especially useful for identifying functional variant sequences. The purpose of screening such combinatorial libraries may be to generate, for example, soluble polypeptide variants which can act as antagonists of EphB4, EphrinB2, or both. Combinatorially-derived variants can be generated which have a selective potency relative to a naturally occurring soluble polypeptide. Likewise, mutagenesis can give rise to variants which have intracellular half-lives dramatically different than the corresponding wild-type soluble polypeptide. For example, the altered protein can be rendered either more stable or less stable to proteolytic degradation or other cellular process which result in destruction of, or otherwise inactivation of the protein of interest (e.g., a soluble polypeptide). Such variants, and the genes which encode them, can be utilized to alter the subject soluble polypeptide levels by modulating their half-life. For instance, a short half-life can give rise to more transient biological effects and, when part of an inducible expression system, can allow tighter control of recombinant soluble polypeptide levels within the cell. As above, such proteins, and particularly their recombinant nucleic acid constructs, can be used in gene therapy protocols.

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential soluble polypeptide sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, soluble polypeptide variants (e.g., the antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the subject soluble polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the subject soluble polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, the subject soluble polypeptides of the invention include a small molecule such as a peptide and a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the EphB4 or EphrinB2 soluble polypeptides.

In certain embodiments, the soluble polypeptides of the invention may further comprise post-translational modifications. Exemplary post-translational protein modification include phosphorylation, acetylation, methylation, ADP-ribosylation, ubiquitination, glycosylation, carbonylation, sumoylation, biotinylation or addition of a polypeptide side chain or of a hydrophobic group. As a result, the modified soluble polypeptides may contain non-amino acid elements, such as lipids, poly- or mono-saccharide, and phosphates. Effects of such non-amino acid elements on the functionality of a soluble polypeptide may be tested for its antagonizing role in EphB4 or EphrinB2 function, e.g, inhibitory effect on viral infection.

In one specific embodiment of the present invention, modified forms of the subject soluble polypeptides comprise linking the subject soluble polypeptides to nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. Examples of the modified polypeptide of the invention include PEGylated soluble EphrinB2 and PEGylated soluble EphB4.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH (1), where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini, C., et al., Bioconjugate Chem. 6 (1995) 62-69).

PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap. 21 and 22). It is noted that an EphB4 containing a PEG molecule is also known as a conjugated protein, whereas the protein lacking an attached PEG molecule can be referred to as unconjugated.

Any molecular mass for a PEG can be used as practically desired, e.g., from about 1,000 Daltons (Da) to 100,000 Da (n is 20 to 2300), for conjugating to Eph4 or EphrinB2 soluble peptides. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

In a specific embodiment of the invention, an EphB4 polypeptide is covalently linked to one poly(ethylene glycol) group of the formula: —CO—(CH₂)ₓ—(OCH₂CH₂)ₘ—OR, with the —CO (i.e. carbonyl) of the poly(ethylene glycol) group forming an amide bond with one of the amino groups of EphB4; R being lower alkyl; x being 2 or 3; m being from about 450 to about 950; and n and m being chosen so that the molecular weight of the conjugate minus the EphB4 protein is from about 10 to 40 kDa. In one embodiment, an EphB4 E-amino group of a lysine is the available (free) amino group.

The above conjugates may be more specifically presented by formula (II): P—NHCO— (CH₂)ₙ—(OCH₂CH₂)ₘ—OR (II), wherein P is the group of an EphB4 protein as described herein, (i.e. without the amino group or amino groups which form an amide linkage with the carbonyl shown in formula (II); and wherein R is lower alkyl; x is 2 or 3; m is from about 450 to about 950 and is chosen so that the molecular weight of the conjugate minus the EphB4 protein is from about 10 to about 40 kDa. As used herein, the given ranges of “m” have an orientational meaning. The ranges of “m” are determined in any case, and exactly, by the molecular weight of the PEG group.

One skilled in the art can select a suitable molecular mass for PEG, e.g., based on how the pegylated EphB4 will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 10: 91-114 (1993).

In one embodiment of the invention, PEG molecules may be activated to react with amino groups on EphB4, such as with lysines (Bencham C. O. et al., Anal. Biochem., 131, 25 (1983); Veronese, F. M. et al., Appl. Biochem., 11, 141 (1985).; Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)).

In one specific embodiment, carbonate esters of PEG are used to form the PEG-EphB4 conjugates. N,N′-disuccinimidylcarbonate (DSC) may be used in the reaction with PEG to form active mixed PEG-succinimidyl carbonate that may be subsequently reacted with a nucleophilic group of a linker or an amino group of EphB4 (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively.

In one embodiment, additional sites for PEGylation are introduced by site-directed mutagenesis by introducing one or more lysine residues. For instance, one or more arginine residues may be mutated to a lysine residue. In another embodiment, additional PEGylation sites are chemically introduced by modifying amino acids on EphB4. In one specific embodiment, carboxyl groups in EphB4 are conjugated with diaminobutane, resulting in carboxyl amidation (see Li et al., Anal Biochem. 2004; 330(2):264-71). This reaction may be catalyzed by 1-ethyl-3-(3-dimethylaminopropyl) carbodiimide, a water-soluble carbodiimide. The resulting amides can then conjugated to PEG.

PEGylation of EphB4 can be performed according to the methods of the state of the art, for example by reaction of EphB4 with electrophilically active PEGs (supplier: Shearwater Corp., USA, www.shearwatercorp.com). Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C., et al., Bioconjugate Chem. 6 (1995) 62-69). Such methods may used to PEGylated at an ε-amino group of an EphB4 lysine or the N-terminal amino group of EphB4.

In another embodiment, PEG molecules may be coupled to sulfhydryl groups on EphB4 (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups.

In some embodiments where PEG molecules are conjugated to cysteine residues on EphB4, the cysteine residues are native to Eph4, whereas in other embodiments, one or more cysteine residues are engineered into EphB4. Mutations may be introduced into an EphB4 coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of EphB4 an EphB2, given that the crystal structure of EphB2 has been solved (see Himanen et al., Nature. (2001) 20-27; 414(6866):933-8) and thus the surface-exposed residues identified. In one embodiment, cysteine residues are introduced into EphB4 at or near the N- and/or C-terminus, or within loop regions. Loop regions may be identified by comparing the EphB4 sequence to that of EphB2.

In some embodiments, the pegylated EphB4 comprises a PEG molecule covalently attached to the alpha amino group of the N-terminal amino acid. Site specific N-terminal reductive amination is described in Pepinsky et al., (2001) JPET, 297, 1059, and U.S. Pat. No. 5,824,784. The use of a PEG-aldehyde for the reductive amination of a protein utilizing other available nucleophilic amino groups is described in U.S. Pat. No. 4,002,531, in Wieder et al., (1979) J. Biol. Chem. 254, 12579, and in Chamow et al., (1994) Bioconjugate Chem. 5, 133.

In another embodiment, pegylated EphB4 comprises one or more PEG molecules covalently attached to a linker, which in turn is attached to the alpha amino group of the amino acid residue at the N-terminus of EphB4. Such an approach is disclosed in U.S. Patent Publication No. 2002/0044921 and in WO94/01451.

In one embodiment, EphB4 is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5):1005-1009.

Monopegylation of EphB4 can also be produced according to the general methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of EphB4 (or EphrinB2) to activated PEG in the conjugation reaction can be from about 1:0.5 to 1:50, between from about 1:1 to 1:30, or from about 1:5 to 1:15. Various aqueous buffers can be used in the present method to catalyze the covalent addition of PEG to EphB4. In one embodiment, the pH of a buffer used is from about 7.0 to 9.0. In another embodiment, the pH is in a slightly basic range, e.g., from about 7.5 to 8.5. Buffers having a pKa close to neutral pH range may be used, e.g., phosphate buffer.

In one embodiment, the temperature range for preparing a mono-PEG-EphB4 is from about 4° C. to 40° C., or from about 18° C. to 25° C. In another embodiment, the temperature is room temperature.

The pegylation reaction can proceed from 3 to 48 hours, or from 10 to 24 hours. The reaction can be monitored using SE-HPLC to distinguish EphB4, mono-PEG-EphB4 and poly-PEG-EphB4. It is noted that mono-PEG-EphB4 forms before di-PEG-EphB4. When the mono-PEG-EphB4 concentration reaches a plateau, the reaction can be terminated by adding a quenching agent to react with unreacted PEG. In some embodiments, the quenching agent is a free amino acid, such as glycine, cysteine or lysine.

Conventional separation and purification techniques known in the art can be used to purify pegylated EphB4 or EphrinB2 products, such as size exclusion (e.g. gel filtration) and ion exchange chromatography. Products may also be separated using SDS-PAGE. Products that may be separated include mono-, di-, tri- poly- and un-pegylated EphB4, as well as free PEG. The percentage of mono-PEG conjugates can be controlled by pooling broader fractions around the elution peak to increase the percentage of mono-PEG in the composition. About ninety percent mono-PEG conjugates represents a good balance of yield and activity. Compositions in which, for example, at least ninety-two percent or at least ninety-six percent of the conjugates are mono-PEG species may be desired. In an embodiment of this invention the percentage of mono-PEG conjugates is from ninety percent to ninety-six percent.

In one embodiment, pegylated EphB4 proteins of the invention contain one, two or more PEG moieties. In one embodiment, the PEG moiety(ies) are bound to an amino acid residue which is on the surface of the protein and/or away from the surface that contacts EphrinB2. In one embodiment, the combined or total molecular mass of PEG in PEG-EphB4 is from about 3,000 Da to 60,000 Da, optionally from about 10,000 Da to 36,000 Da. In a one embodiment, the PEG in pegylated EphB4 is a substantially linear, straight-chain PEG.

In one embodiment of the invention, the PEG in pegylated EphB4 or EphrinB2 is not hydrolyzed from the pegylated amino acid residue using a hydroxylamine assay, e.g., 450 mM hydroxylamine (pH 6.5) over 8 to 16 hours at room temperature, and is thus stable. In one embodiment, greater than 80% of the composition is stable mono-PEG-EphB4, more preferably at least 90%, and most preferably at least 95%.

In another embodiment, the pegylated EphB4 proteins of the invention will preferably retain at least 25%, 50%, 60%, 70%, 80%, 85%, 90%, 95% or 100% of the biological activity associated with the unmodified protein. In one embodiment, biological activity refers to its ability to bind to EphrinB2. In one specific embodiment, the pegylated EphB4 protein shows an increase in binding to EphrinB2 relative to unpegylated EphB4.

In a preferred embodiment, the PEG-EphB4 has a half-life (t_{1/2}) which is enhanced relative to the half-life of the unmodified protein. Preferably, the half-life of PEG-EphB4 is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified EphB4 protein. In some embodiments, the protein half-life is determined in vitro, such as in a buffered saline solution or in serum. In other embodiments, the protein half-life is an in vivo half life, such as the half-life of the protein in the serum or other bodily fluid of an animal.

In certain aspects, functional variants or modified forms of the subject soluble polypeptides include fusion proteins having at least a portion of the soluble polypeptide and one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Another fusion domain well known in the art is green fluorescent protein (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation.

In certain embodiments, the soluble polypeptides of the present invention contain one or more modifications that are capable of stabilizing the soluble polypeptides. For example, such modifications enhance the in vitro half life of the soluble polypeptides, enhance circulatory half life of the soluble polypeptides or reducing proteolytic degradation of the soluble polypeptides.

In certain embodiments, the soluble polypeptides of the present invention may be fused to other therapeutic proteins or to other proteins such as Fc or serum albumin for pharmacokinetic purposes. See for example U.S. Pat. Nos. 5,766,883 and 5,876,969, both of which are incorporated by reference. In some embodiments, soluble peptides of the present invention are fused to Fc variants. In a specific embodiment, the soluble polypeptide is fused to an Fc variant which does not homodimerize, such as one lacking the cysteine residues which form cysteine bonds with other Fc chains.

In some embodiments, the modified proteins of the invention comprise fusion proteins with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

In one embodiment, the class of immunoglobulin from which the heavy chain constant region is derived is IgG (Igγ) (γ subclasses 1, 2, 3, or 4), including nucleotide and amino acid sequences of human Fcγ-1 and murine Fcγ-2a. Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM.

Furthermore, it is contemplated that substitution or deletion of amino acids within the immunoglobulin heavy chain constant regions may be useful in the practice of the invention. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In a specific embodiment of the present invention, the modified forms of the subject soluble polypeptides are fusion proteins having at least a portion of the soluble polypeptide (e.g., an ectodomain of EphrinB2 or EphB4) and a stabilizing domain such as albumin. As used herein, “albumin” refers collectively to albumin protein or amino acid sequence, or an albumin fragment or variant, having one or more functional activities (e.g., biological activities) of albumin. In particular, “albumin” refers to human albumin or fragments thereof (see EP 201 239, EP 322 094 WO 97/24445, WO95/23857) especially the mature form of human albumin, or albumin from other vertebrates or fragments thereof, or analogs or variants of these molecules or fragments thereof.

The present invention describes that such fusion proteins are more stable relative to the corresponding wildtype soluble protein. For example, the subject soluble polypeptide (e.g., an ectodomain of EphrinB2 or EphB4) can be fused with human serum albumin (HSA), bovine serum albumin (BSA), or any fragment of an albumin protein which has stabilization activity. Such stabilizing domains include human serum albumin (HSA) and bovine serum albumin (BSA).

In particular, the albumin fusion proteins of the invention may include naturally occurring polymorphic variants of human albumin and fragments of human albumin (See WO95/23857), for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the albumin fusion protein may be from a different animal than the EphB4.

In some embodiments, the albumin protein portion of an albumin fusion protein corresponds to a fragment of serum albumin. Fragments of serum albumin polypeptides include polypeptides having one or more residues deleted from the amino terminus or from the C-terminus. Generally speaking, an HA fragment or variant will be at least 100 amino acids long, preferably at least 150 amino acids long. The HA variant may consist of or alternatively comprise at least one whole domain of HA. Domains, with reference to SEQ ID NO: 18 in U.S. Patent Publication No. 2004/0171123, are as follows: domains 1 (amino acids 1-194), 2 (amino acids 195-387), 3 (amino acids 388-585), 1+2 (1-387), 2+3 (195-585) or 1+3 (amino acids 1-194 + amino acids 388-585). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

In one embodiment, the EphB4-HSA fusion has one EphB4 soluble polypeptide linked to one HSA molecule, but other conformations are within the invention. For example, EphB4-HSA fusion proteins can have any of the following formula: R₁-L-R₂; R₂-L-R₁; R₁-L-R₂-L-R₁; or R₂-L-R₁-L-R₂; R₁-R₂; R₂-R₁; R₁-R₂-R₁; or R₂-R₁-R₂; wherein R₁ is a soluble EphB4 sequence, R₂ is HSA, and L is a peptide linker sequence.

In a specific embodiment, the EphB4 and HSA domains are linked to each other, preferably via a linker sequence, which separates the EphB4 and HSA domains by a distance sufficient to ensure that each domain properly folds into its secondary and tertiary structures. Preferred linker sequences (1) should adopt a flexible extended conformation, (2) should not exhibit a propensity for developing an ordered secondary structure which could interact with the functional EphB4 and HSA domains, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence.

In a specific embodiment, a linker sequence length of about 20 amino acids can be used to provide a suitable separation of functional protein domains, although longer or shorter linker sequences may also be used. The length of the linker sequence separating EphB4 and HSA can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. Amino acid sequences useful as linkers of EphB4 and HSA include, but are not limited to, (SerGly₄)y wherein y is greater than or equal to 8, or Gly₄SerGly₅Ser. A preferred linker sequence has the formula (SerGly₄)₄. Another preferred linker has the sequence ((Ser-Ser-Ser-Ser-Gly)3-Ser-Pro).

In one embodiment, the polypeptides of the present invention and HSA proteins are directly fused without a linker sequence. In preferred embodiments, the C-terminus of a soluble EphB4 polypeptide can be directly fused to the N-terminus of HSA or the C-terminus of HSA can be directly fused to the N-terminus of soluble EphB4.

In some embodiments, the immunogenicity of the fusion junction between HSA and EphB4 may be reduced the by identifying a candidate T-cell epitope within a junction region spanning a fusion protein and changing an amino acid within the junction region as described in U.S. Patent Publication No. 2003/0166877.

In certain embodiments, soluble polypeptides (unmodified or modified) of the invention can be produced by a variety of art-known techniques. For example, such soluble polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W.H. Freeman and Company, New York (1992). In addition, automated peptide synthesizers are commercially available (e.g., Advanced ChemTech Model 396; Milligen/Biosearch 9600). Alternatively, the soluble polypeptides, fragments or variants thereof may be recombinantly produced using various expression systems as is well known in the art (also see below).

III. Nucleic Acids Encoding Soluble Polypeptides

In certain aspects, the invention relates to isolated and/or recombinant nucleic acids encoding an EphB4 or EphrinB2 soluble polypeptide. The subject nucleic acids may be single-stranded or double-stranded, DNA or RNA molecules. These nucleic acids are useful as therapeutic agents. For example, these nucleic acids are useful in making recombinant soluble polypeptides which are administered to a cell or an individual as therapeutics. Alternative, these nucleic acids can be directly administered to a cell or an individual as therapeutics such as in gene therapy.

In certain embodiments, the invention provides isolated or recombinant nucleic acid sequences that are at least 80%, 85%, 90%, 95%, 97%, 98%, 99% or 100% identical to a region of the nucleotide sequence depicted in SEQ ID NOs: 8-9. One of ordinary skill in the art will appreciate that nucleic acid sequences complementary to the subject nucleic acids, and variants of the subject nucleic acids are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleotide sequences that hybridize under highly stringent conditions to the nucleotide sequence depicted in SEQ ID NOs: 8-9, or complement sequences thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the subject nucleic acids due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

In certain embodiments, the recombinant nucleic acids of the invention may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspect of the invention, the subject nucleic acid is provided in an expression vector comprising a nucleotide sequence encoding an EphB4 or EphrinB2 soluble polypeptide and operably linked to at least one regulatory sequence. Regulatory sequences are art-recognized and are selected to direct expression of the soluble polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a soluble polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This invention also pertains to a host cell transfected with a recombinant gene including a coding sequence for one or more of the subject soluble polypeptide. The host cell may be any prokaryotic or eukaryotic cell. For example, a soluble polypeptide of the invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject soluble polypeptides. For example, a host cell transfected with an expression vector encoding an EphB4 soluble polypeptide can be cultured under appropriate conditions to allow expression of the EphB4 soluble polypeptide to occur. The EphB4 soluble polypeptide may be secreted and isolated from a mixture of cells and medium containing the soluble polypeptides. Alternatively, the soluble polypeptides may be retained cytoplasmically or in a membrane fraction and the cells harvested, lysed and the protein isolated. A cell culture includes host cells, media and other byproducts. Suitable media for cell culture are well known in the art. The soluble polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the soluble polypeptides. In a preferred embodiment, the soluble polypeptide is a fusion protein containing a domain which facilitates its purification.

A recombinant nucleic acid of the invention can be produced by ligating the cloned gene, or a portion thereof, into a vector suitable for expression in either prokaryotic cells, eukaryotic cells (yeast, avian, insect or mammalian), or both. Expression vehicles for production of a recombinant soluble polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant SLC5A8 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

IV. Nucleic Acid Therapeutic Agents

This disclosure relates to nucleic acid therapeutic agents and methods for inhibiting or reducing gene expression of ephrinB2. By “inhibit” or “reduce,” it is meant that the expression of the gene, or level of nucleic acids or equivalent nucleic acids encoding one or more proteins or protein subunits, such as EphrinB2, is reduced below that observed in the absence of the nucleic acid therapeutic agents of the disclosure. By “gene,” it is meant a nucleic acid that encodes an RNA, for example, nucleic acid sequences including but not limited to structural genes encoding a polypeptide.

As used herein, the term “nucleic acid therapeutic agent” or “nucleic acid agent” or “nucleic acid compound” refers to any nucleic acid-based compound that contains nucleotides and has a desired effect on a target gene. The nucleic acid therapeutic agents can be single-, double-, or multiple-stranded, and can comprise modified or unmodified nucleotides or non-nucleotides or various mixtures, and combinations thereof. Examples of nucleic acid therapeutic agents of the disclosure include, but are not limited to, antisense nucleic acids, dsRNA, siRNA, and enzymatic nucleic acid compounds.

In one embodiment, the disclosure features one or more nucleic acid therapeutic agents that independently or in combination modulate expression of the EphrinB2 gene encoding an EphrinB2 protein (e.g., Genbank Accession No.: NP_{—}004084).

A. Antisense Nucleic Acids

In certain embodiments, the disclosure relates to antisense nucleic acids. By “antisense nucleic acid,” it is meant a non-enzymatic nucleic acid compound that binds to a target nucleic acid by means of RNA-RNA, RNA-DNA or RNA-PNA (protein nucleic acid) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can form a loop and binds to a substrate nucleic acid which forms a loop. Thus, an antisense molecule can be complementary to two (or more) non-contiguous substrate sequences, or two (or more) non-contiguous sequence portions of an antisense molecule can be complementary to a target sequence, or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

In addition, antisense DNA can be used to target nucleic acid by means of DNA-RNA interactions, thereby activating RNase H, which digests the target nucleic acid in the duplex. The antisense oligonucleotides can comprise one or more RNAse H activating region, which is capable of activating RNAse H to cleave a target nucleic acid. Antisense DNA can be synthesized chemically or expressed via the use of a single stranded DNA expression vector or equivalent thereof. By “RNase H activating region” is meant a region (generally greater than or equal to 4-25 nucleotides in length, preferably from 5-11 nucleotides in length) of a nucleic acid compound capable of binding to a target nucleic acid to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to a nucleic acid compound-target nucleic acid complex and cleaves the target nucleic acid sequence.

The RNase H activating region comprises, for example, phosphodiester, phosphorothioate, phosphorodithioate, 5′-thiophosphate, phosphoramidate or methylphosphonate backbone chemistry, or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant disclosure.

Thus, the antisense nucleic acids of the disclosure include natural-type oligonucleotides and modified oligonucleotides including phosphorothioate-type oligodeoxyribonucleotides, phosphorodithioate-type oligodeoxyribonucleotides, methylphosphonate-type oligodeoxyribonucleotides, phosphoramidate-type oligodeoxyribonucleotides, H-phosphonate-type oligodeoxyribonucleotides, triester-type oligodeoxyribonucleotides, alpha-anomer-type oligodeoxyribonucleotides, peptide nucleic acids, other artificial nucleic acids, and nucleic acid-modified compounds.

Other modifications include those which are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as arabinose instead of ribose, or a 3′,5′-substituted oligonucleotide having a sugar which, at both its 3′ and 5′ positions is attached to a chemical group other than a hydroxyl group (at its 3′ position) and other than a phosphate group (at its 5′ position).

Other examples of modifications to sugars include modifications to the 2′ position of the ribose moiety which include but are not limited to 2′-O-substituted with an —O-lower alkyl group containing 1-6 saturated or unsaturated carbon atoms, or with an —O-aryl, or allyl group having 2-6 carbon atoms wherein such —O-alkyl, aryl or allyl group may be unsubstituted or may be substituted, (e.g., with halo, hydroxy, trifluoromethyl cyano, nitro acyl acyloxy, alkoxy, carboxy, carbalkoxyl, or amino groups), or with an amino, or halo group. Nonlimiting examples of particularly useful oligonucleotides of the disclosure have 2′-O-alkylated ribonucleotides at their 3′, 5′, or 31 and 5′ termini, with at least four or five contiguous nucleotides being so modified. Examples of 2′-O-alkylated groups include, but are not limited to, 2′-O-methyl, 2′-O-ethyl, 2′-O-propyl, and 2′-O-butyls.

In certain cases, the synthesis of the natural-type and modified antisense nucleic acids can be carried out with, for example, a 381A DNA synthesizer or 394 DNA/RNA synthesizer manufactured by ABI (Applied Biosystems Inc.) in accordance with the phosphoramidite method (see instructions available from ABI, or F. Eckstein, Oligonucleotides and Analogues: A Practical Approach, IRL Press (1991)). In the phosphoramidite method, a nucleic acid-related molecule is synthesized by condensation between the 3′-terminus of a modified deoxyribonucleoside or modified ribonucleoside and the 5′-terminus of another modified deoxyribonucleoside, modified ribonucleoside, oligo-modified deoxyribonucleotide or oligo-modified-ribonucleotide by use of a reagent containing phosphoramidite protected with a group such as cyanoethyl group. The final cycle of this synthesis is finished to give a product with a protective group (e.g., dimethoxytrityl group) bound to a hydroxyl group at the 5′-terminus of the sugar moiety. The oligomer thus synthesized at room temperature is cleaved off from the support, and its nucleotide and phosphate moieties are deprotected. In this manner, the natural-type oligonucleic acid compound is obtained in a crude form. The phosphorothioate-type nucleic acids can also be synthesized in a similar manner to the above natural type by the phosphoramidite method with the synthesizer from ABI. The procedure after the final cycle of the synthesis is also the same as with the natural type.

The crude nucleic acids (natural type or modified) thus obtained can be purified in a usual manner e.g., ethanol precipitation, or reverse phase chromatography, ion-exchange chromatography and gel filtration chromatography in high performance liquid chromatography (HPLC), supercritical fluid chromatography, and it may be further purified by electrophoresis. A cartridge for reverse phase chromatography, such as tC18-packed SepPak Plus (long body/ENV) (Waters), can also be used. The purity of the natural-type and modified (e.g., phosphorothioate-type) nucleic acids can be analyzed by HPLC.

In certain embodiments, the antisense nucleic acids of the disclosure can be delivered, for example, as an expression plasmid which, when transcribed in the cell, produces RNA which is complementary to at least a unique portion of the cellular mRNA which encodes an EphrinB2 polypeptide. Alternatively, the construct is an oligonucleotide which is generated ex vivo and which, when introduced into the cell causes inhibition of expression by hybridizing with the mRNA and/or genomic sequences encoding an EphrinB2 polypeptide. Such oligonucleotide probes are optionally modified oligonucleotide which are resistant to endogenous nucleases, e.g., exonucleases and/or endonucleases, and are therefore stable in vivo. Exemplary nucleic acid compounds for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

B. dsRNA and RNAi Constructs

In certain embodiments, the disclosure relates to double stranded RNA (dsRNA) and RNAi constructs. The term “dsRNA” as used herein refers to a double stranded RNA molecule capable of RNA interference (RNAi), including siRNA (see for example, Bass, 2001, Nature, 411, 428-429; Elbashir et al., 2001, Nature, 411, 494-498; and Kreutzer et al., PCT Publication No. WO 00/44895; Zernicka-Goetz et al., PCT Publication No. WO 01/36646; Fire, PCT Publication No. WO 99/32619; Plaetinck et al., PCT Publication No. WO 00/01846; Mello and Fire, PCT Publication No. WO 01/29058; Deschamps-Depaillette, PCT Publication No. WO 99/07409; and Li et al., PCT Publication No. WO 00/44914). In addition, RNAi is a term initially applied to a phenomenon observed in plants and worms where double-stranded RNA (dsRNA) blocks gene expression in a specific and post-transcriptional manner. RNAi provides a useful method of inhibiting gene expression in vitro or in vivo.

The term “short interfering RNA,” “siRNA,” or “short interfering nucleic acid,” as used herein, refers to any nucleic acid compound capable of mediating RNAi or gene silencing when processed appropriately be a cell. For example, the siRNA can be a double-stranded polynucleotide molecule comprising self-complementary sense and antisense regions, wherein the antisense region comprises complementarity to a target nucleic acid compound (e.g., EphrinB2). The siRNA can be a single-stranded hairpin polynucleotide having self-complementary sense and antisense regions, wherein the antisense region comprises complementarity to a target nucleic acid compound. The siRNA can be a circular single-stranded polynucleotide having two or more loop structures and a stem comprising self-complementary sense and antisense regions, wherein the antisense region comprises complementarity to a target nucleic acid compound, and wherein the circular polynucleotide can be processed either in vivo or in vitro to generate an active siRNA capable of mediating RNAi. The siRNA can also comprise a single stranded polynucleotide having complementarity to a target nucleic acid compound, wherein the single stranded polynucleotide can further comprise a terminal phosphate group, such as a 5′-phosphate (see for example Martinez et al., 2002, Cell., 110, 563-574), or 5′,3′-diphosphate.

Optionally, the siRNAs of the disclosure contain a nucleotide sequence that hybridizes under physiologic conditions of the cell to the nucleotide sequence of at least a portion of the mRNA transcript for the gene to be inhibited (the “target” gene). The double-stranded RNA need only be sufficiently similar to natural RNA that it has the ability to mediate RNAi. Thus, the disclosure has the advantage of being able to tolerate sequence variations that might be expected due to genetic mutation, strain polymorphism or evolutionary divergence. The number of tolerated nucleotide mismatches between the target sequence and the siRNA sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the siRNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure of dsRNA may be formed by a single self-complementary RNA strand, two complementary RNA strands, or a DNA strand and a complementary RNA strand. Optionally, RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

As described herein, the subject siRNAs are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of the subject dsRNAs (e.g., siRNAs) can be carried out by chemical synthetic methods or by recombinant nucleic acid techniques. Endogenous RNA polymerase of the treated cell may mediate transcription in vivo, or cloned RNA polymerase can be used for transcription in vitro. As used herein, dsRNA or siRNA molecules of the disclosure need not be limited to those molecules containing only RNA, but further encompasses chemically-modified nucleotides and non-nucleotides. For example, the dsRNAs may include modifications to either the phosphate-sugar backbone or the nucleoside, e.g., to reduce susceptibility to cellular nucleases, improve bioavailability, improve formulation characteristics, and/or change other pharmacokinetic properties. To illustrate, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The dsRNAs may be produced enzymatically or by partial/total organic synthesis, any modified ribonucleotide can be introduced by in vitro enzymatic or organic synthesis. Methods of chemically modifying RNA molecules can be adapted for modifying dsRNAs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an dsRNA can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). In certain cases, the dsRNAs of the disclosure lack 2′-hydroxy (2′-OH) containing nucleotides.

In a specific embodiment, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In another specific embodiment, the subject dsRNA can also be in the form of a long double-stranded RNA. For example, the dsRNA is at least 25, 50, 100, 200, 300 or 400 bases. In some cases, the dsRNA is 400-800 bases in length. Optionally, the dsRNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In a further specific embodiment, the dsRNA is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present disclosure provides a recombinant vector having the following unique characteristics: it comprises a viral replicon having two overlapping transcription units arranged in an opposing orientation and flanking a transgene for a dsRNA of interest, wherein the two overlapping transcription units yield both sense and antisense RNA transcripts from the same transgene fragment in a host cell.

C. Enzymatic Nucleic Acid Compounds

In certain embodiments, the disclosure relates to enzymatic nucleic acid compounds. By “enzymatic nucleic acid compound,” it is meant a nucleic acid compound which has complementarity in a substrate binding region to a specified target gene, and also has an enzymatic activity which is active to specifically cleave a target nucleic acid. It is understood that the enzymatic nucleic acid compound is able to intermolecularly cleave a nucleic acid and thereby inactivate a target nucleic acid compound. These complementary regions allow sufficient hybridization of the enzymatic nucleic acid compound to the target nucleic acid and thus permit cleavage. One hundred percent complementarity (identity) is preferred, but complementarity as low as 50-75% can also be useful in this disclosure (see for example Werner and Uhlenbeck, 1995, Nucleic Acids Research, 23, 2092-2096; Hammann et al., 1999, Antisense and Nucleic Acid Drug Dev., 9, 25-31). The enzymatic nucleic acids can be modified at the base, sugar, and/or phosphate groups. As described herein, the term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid compounds with enzymatic activity. The specific enzymatic nucleic acid compounds described in the instant application are not limiting in the disclosure and those skilled in the art will recognize that all that is important in an enzymatic nucleic acid compound of this disclosure is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

Several varieties of naturally-occurring enzymatic nucleic acids are currently known. Each can catalyze the hydrolysis of nucleic acid phosphodiester bonds in trans (and thus can cleave other nucleic acid compounds) under physiological conditions. In general, enzymatic nucleic acids act by first binding to a target nucleic acid. Such binding occurs through the target binding portion of a enzymatic nucleic acid which is held in close proximity to an enzymatic portion of the molecule that acts to cleave the target nucleic acid. Thus, the enzymatic nucleic acid first recognizes and then binds a target nucleic acid through complementary base-pairing, and once bound to the correct site, acts enzymatically to cut the target nucleic acid. Strategic cleavage of such a target nucleic acid will destroy its ability to direct synthesis of an encoded protein. After an enzymatic nucleic acid has bound and cleaved its nucleic acid target, it is released from that nucleic acid to search for another target and can repeatedly bind and cleave new targets.

In a specific embodiment, the subject enzymatic nucleic acid is a ribozyme designed to catalytically cleave an mRNA transcripts to prevent translation of mRNA (see, e.g., PCT International Publication WO90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNAs have the following sequence of two bases: 5′-UG-3′. The construction and production of hammerhead ribozymes is well known in the art and is described more fully in Haseloff and Gerlach, 1988, Nature, 334:585-591. The ribozymes of the present disclosure also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In another specific embodiment, the subject enzymatic nucleic acid is a DNA enzyme. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

In certain embodiments, the nucleic acid therapeutic agents of the disclosure can be between 12 and 200 nucleotides in length. In one embodiment, exemplary enzymatic nucleic acid compounds of the disclosure are between 15 and 50 nucleotides in length, including, for example, between 25 and 40 nucleotides in length (for example see Jarvis et al., 1996, J. Biol. Chem., 271, 29107-29112). In another embodiment, exemplary antisense molecules of the disclosure are between 15 and 75 nucleotides in length, including, for example, between 20 and 35 nucleotides in length (see for example Woolf et al., 1992, PNAS., 89, 7305-7309; Milner et al., 1997, Nature Biotechnology, 15, 537-541). In another embodiment, exemplary siRNAs of the disclosure are between 20 and 27 nucleotides in length, including, for example, between 21 and 23 nucleotides in length. Those skilled in the art will recognize that all that is required is that the subject nucleic acid therapeutic agent be of length and conformation sufficient and suitable for catalyzing a reaction contemplated herein. The length of the nucleic acid therapeutic agents of the instant disclosure is not limiting within the general limits stated.

V. Target Sites

Targets for useful nucleic acid compounds of the disclosure (e.g., antisense nucleic acids, dsRNA, and enzymatic nucleic acid compounds) can be determined as disclosed in Draper et al., 30 WO 93/23569; Sullivan et al., WO 93/23057; Thompson et al., WO 94/02595; Draper et al., WO 95/04818; McSwiggen et al., U.S. Pat. No. 5,525,468. Other examples include the following PCT applications inactivation of expression of disease-related genes: WO 95/23225, WO 95/13380, WO 94/02595. Rather than repeat the guidance provided in those documents here, below are provided specific examples of such methods, not limiting to those in the art.

Enzymatic nucleic acid compounds, siRNA and antisense to such targets are designed as described in those applications and synthesized to be tested in vitro and in vivo, as also described. For examples, the sequence of human EphrinB2 RNAs are screened for optimal nucleic acid target sites using a computer-folding algorithm. Potential nucleic acid binding/cleavage sites are identified. For example, for enzymatic nucleic acid compounds of the disclosure, the nucleic acid compounds are individually analyzed by computer folding (Jaeger et al., 1989 Proc. Natl. Acad. Sci. USA, 86, 7706) to assess whether the sequences fold into the appropriate secondary structure. Those nucleic acid compounds with unfavorable intramolecular interactions such as between the binding arms and the catalytic core can be eliminated from consideration.

The subject nucleic acid (e.g., antisense, RNAi, and/or enzymatic nucleic acid compound) binding/cleavage sites are identified and are designed to anneal to various sites in the nucleic acid target (e.g., EphrinB2). The binding arms of enzymatic nucleic acid compounds of the disclosure are complementary to the target site sequences described above. Antisense and RNAi sequences are designed to have partial or complete complementarity to the nucleic acid target. The nucleic acid compounds can be chemically synthesized. The method of synthesis used follows the procedure for normal DNA/RNA synthesis as described below and in Usman et al., 1987 J Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211, 3-19.

Additionally, it is expected that nucleic acid therapeutic agents having a CpG motif are at an increased likelihood of causing a non-specific immune response. Generally, CpG motifs include a CG (Cytosine-Guanosine) sequence adjacent to one or more purines in the 5′ direction and one or more pyrimidines in the 3′ direction. Lists of known CpG motifs are available in the art. Preferred nucleic acid therapeutics will be selected so as to have a selective effect on the target gene (possibly affecting other closely related genes) without triggering a generalized immune response. By avoiding nucleic acid therapeutics having a CpG motif, it is possible to decrease the likelihood that a particular nucleic acid will trigger an immune response.

VI. Synthesis of Nucleic Acid Therapeutic Agents

Synthesis of nucleic acids greater than 100 nucleotides in length is difficult using automated methods, and the therapeutic cost of such molecules is prohibitive. In this disclosure, small nucleic acid motifs (small refers to nucleic acid motifs less than about 100 nucleotides in length, preferably less than about 80 nucleotides in length, and more preferably less than about 50 nucleotides in length (e.g., antisense oligonucleotides, enzymatic nucleic acids, and RNAi constructs) are preferably used for exogenous delivery. The simple structure of these molecules increases the ability of the nucleic acid to invade targeted regions of RNA structure.

Exemplary molecules of the instant disclosure are chemically synthesized, and others can similarly be synthesized. To illustrate, oligonucleotides (e.g., DNA) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer with a 2.5 min coupling step for 2′-O-methylated nucleotides and a 45 sec coupling step for 2′-deoxy nucleotides. Alternatively, syntheses can be performed on a 96-well plate synthesizer, such as the instrument produced by Protogene (Palo Alto, Calif.) with minimal modification to the cycle.

Optionally, the nucleic acid compounds of the present disclosure can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

Preferably, the nucleic acid compounds of the present disclosure are modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water

VII. Optimizing Activity of the Nucleic Acid Compounds

Nucleic acid compounds with modifications (e.g., base, sugar and/or phosphate) can prevent their degradation by serum ribonucleases and thereby increase their potency. There are several examples in the art describing sugar, base and phosphate modifications that can be introduced into nucleic acid compounds with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid compounds have been extensively described in the art (see Eckstein et al., PCT Publication No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. PCT Publication No. WO 93/15187; Sproat, U.S. Pat. No. 5,334,711 and Beigelman et al., 1995, J. Biol. Chem., 270, 25702; Beigelman et al., PCT publication No. WO 97/26270; Beigelman et al., U.S. Pat. No. 5,716,824; Usman et al., U.S. Pat. No. 5,627,053; Woolf et al., PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010). Similar modifications can be used to modify the nucleic acid compounds of the instant disclosure.

While chemical modification of oligonucleotide internucleotide linkages with phosphorothioate, phosphorothioate, and/or 5′-methylphosphonate linkages improves stability, an over-abundance of these modifications can cause toxicity. Therefore, the amount of these internucleotide linkages should be evaluated and appropriately minimized when designing the nucleic acid compounds. The reduction in the concentration of these linkages should lower toxicity resulting in increased efficacy and higher specificity of these molecules.

In one embodiment, nucleic acid compounds of the disclosure include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example, Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid compounds of the disclosure results in both enhanced affinity and specificity to nucleic acid targets. In another embodiment, nucleic acid compounds of the disclosure include one or more LNA (locked nucleic acid) nucleotides such as a 2′,4′-C methylene bicyclo nucleotide (see for example Wengel et al., PCT Publication Nos. WO 00/66604 and WO 99/14226).

In another embodiment, the disclosure features conjugates and/or complexes of nucleic acid compounds targeting EphrinB2. Such conjugates and/or complexes can be used to facilitate delivery of nucleic acid compounds into a biological system, such as cells. The conjugates and complexes provided by the instant disclosure can impart therapeutic activity by transferring therapeutic compounds across cellular membranes, altering the pharmacokinetics, and/or modulating the localization of nucleic acid compounds of the disclosure.

Therapeutic nucleic acid compounds, such as the molecules described herein, delivered exogenously are optimally stable within cells until translation of the target RNA has been inhibited long enough to reduce the levels of the undesirable protein. This period of time varies between hours to days depending upon the disease state. These nucleic acid compounds should be resistant to nucleases in order to function as effective intracellular therapeutic agents. Improvements in the chemical synthesis of nucleic acid compounds described in the instant disclosure and in the art have expanded the ability to modify nucleic acid compounds by introducing nucleotide modifications to enhance their nuclease stability as described above.

In another aspect the nucleic acid compounds comprise a 5′ and/or a 3′-cap structure. By “cap structure,” it is meant chemical modifications, which have been incorporated at either terminus of the oligonucleotide (see for example Wincott et al., WO 97/26270). These terminal modifications protect the nucleic acid compound from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al, PCT publication No. WO 97/26270). In other non-limiting examples, the 3′-cap includes, for example, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threopentofuranosy nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925).

VIII. Antibodies

In certain aspects, the present invention provides antibodies against EphrinB2 that inhibit interaction of viruses with EphrinB2. Preferably, the antibody binds to an extracellular domain of EphrinB2. It is understood that antibodies of the invention may be polyclonal or monoclonal; intact or truncated, e.g., F(ab′)2, Fab, Fv; xenogeneic, allogeneic, syngeneic, or modified forms thereof, e.g., humanized, chimeric, etc.

For example, by using immunogens derived from an EphrinB2 polypeptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of the peptide. (e.g., a polypeptide or an antigenic fragment which is capable of eliciting an antibody response, or a fusion protein). Techniques for conferring immunogenicity on a protein or peptide include conjugation to carriers or other techniques well known in the art. An immunogenic portion of an EphrinB2 polypeptide can be administered in the presence of adjuvant. The progress of immunization can be monitored by detection of antibody titers in plasma or serum. Standard ELISA or other immunoassays can be used with the immunogen as antigen to assess the levels of antibodies. In one embodiment, antibodies of the invention are specific for the extracellular portion of the EphrinB2 protein. In another embodiment, antibodies of the invention are specific for the intracellular portion or the transmembrane portion of the EphrinB2 protein. In a further embodiment, antibodies of the invention are specific for the extracellular portion of the EphrinB2 protein.

Following immunization of an animal with an antigenic preparation of an EphrinB2 polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an EphrinB2 polypeptide and monoclonal antibodies isolated from a culture comprising such hybridoma cells.

The term “antibody” as used herein is intended to include fragments thereof which are also specifically reactive with an EphrinB2 polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an EphrinB2 polypeptide conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. In preferred embodiments, the antibodies further comprise a label attached thereto and able to be detected (e.g., the label can be a radioisotope, fluorescent compound, enzyme or enzyme co-factor).

In certain preferred embodiments, an antibody of the invention is a monoclonal antibody, and in certain embodiments the invention makes available methods for generating novel antibodies. For example, a method for generating a monoclonal antibody that binds specifically to an EphrinB2 polypeptide may comprise administering to a mouse an amount of an immunogenic composition comprising the EphrinB2 polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the EphrinB2 polypeptide. Once obtained, a hybridoma can be propagated in a cell culture, optionally in culture conditions where the hybridoma-derived cells produce the monoclonal antibody that binds specifically to the EphrinB2 polypeptide. The monoclonal antibody may be purified from the cell culture.

IX. Methods of Treatment

In certain embodiments, the present invention provides methods of preventing or treating viral infections in humans and animals by use of the therapeutic agents relating to EphrinB2 or EphB4. For example, the polypeptide agents disclosed herein may be useful in treating or preventing viral infections caused by viruses of the Paramyxoviridae and preferably members of the genus Henipavirus (e.g., NiV, HeV), and particularly those that bind to EphrinB2. These methods are particularly aimed at therapeutic and prophylactic treatments of susceptible animals (e.g., horses, sheep, pigs, cattle, and more particularly, humans).

In certain embodiments, the present invention provides methods of inhibiting membrane fusion between a virus of the family Paramyxoviridae (e.g, a Henipavirus) and a target cell (e.g, an endothelial cell) by use of the therapeutic agents relating to EphrinB2 or EphB4.

X. Methods of Administration and Pharmaceutical Compositions

In certain embodiments, the subject polypeptide therapeutic agents (e.g., soluble polypeptides or antibodies) of the present invention are formulated with a pharmaceutically acceptable carrier. Such therapeutic agents can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject polypeptide therapeutic agents include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of antiviral therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject polypeptide therapeutic agent as an active ingredient.

In solid dosage forms for oral administration (capsules, tablets, pills, dragees, powders, granules, and the like), one or more polypeptide therapeutic agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such is water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Pharmaceutical compositions suitable for parenteral administration may comprise one or more polypeptide therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal or rectally administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

In other embodiments, the polypeptide therapeutic agents of the instant invention can be expressed within cells from eukaryotic promoters. For example, a soluble polypeptide of EphB4 or EphrinB2 can be expressed in eukaryotic cells from an appropriate vector. The vectors are preferably DNA plasmids or viral vectors. Viral vectors can be constructed based on, but not limited to, adeno-associated virus, retrovirus, adenovirus, or alphavirus. Preferably, the vectors stably introduced in and persist in target cells. Alternatively, viral vectors can be used that provide for transient expression. Such vectors can be repeatedly administered as necessary. Delivery of vectors encoding the subject polypeptide therapeutic agent can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

Methods for delivering the subject nucleic acid compounds are known in the art (see, e.g., Akhtar et al., 1992, Trends Cell Bio., 2, 139; and Delivery Strategies for Antisense Oligonucleotide Therapeutics, ed. Akhtar, 1995; Sullivan et al., PCT Publication No. WO 94/02595). These protocols can be utilized for the delivery of virtually any nucleic acid compound. Nucleic acid compounds can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to, oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT Publication No. WO99/05094, and Klimuk et al., PCT Publication No. WO99/04819.

In other embodiments, certain of the nucleic acid compounds of the instant disclosure can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; propulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45). Those skilled in the art realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the disclosure, RNA molecules of the present disclosure are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid compounds are delivered as described above, and persist in target cells. Alternatively, viral vectors can be used that provide for transient expression of nucleic acid compounds. Such vectors can be repeatedly administered as necessary. Once expressed, the nucleic acid compound binds to the target mRNA. Delivery of nucleic acid compound expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect, the disclosure contemplates an expression vector comprising a nucleic acid sequence encoding at least one of the nucleic acid compounds of the instant disclosure. The nucleic acid sequence is operably linked in a manner which allows expression of the nucleic acid compound of the disclosure. For example, the disclosure features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant disclosure; and wherein said sequence is operably linked to said initiation region and said termination region, in a manner which allows expression and/or delivery of said nucleic acid compound. The vector can optionally include an open reading frame (ORF) for a protein operably linked on the 5′ side or the 3′-side of the sequence encoding the nucleic acid catalyst of the disclosure; and/or an intron (intervening sequences).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Soluble Derivatives of the Extracellular Domains of Human EphrinB2 and EphB4 Proteins

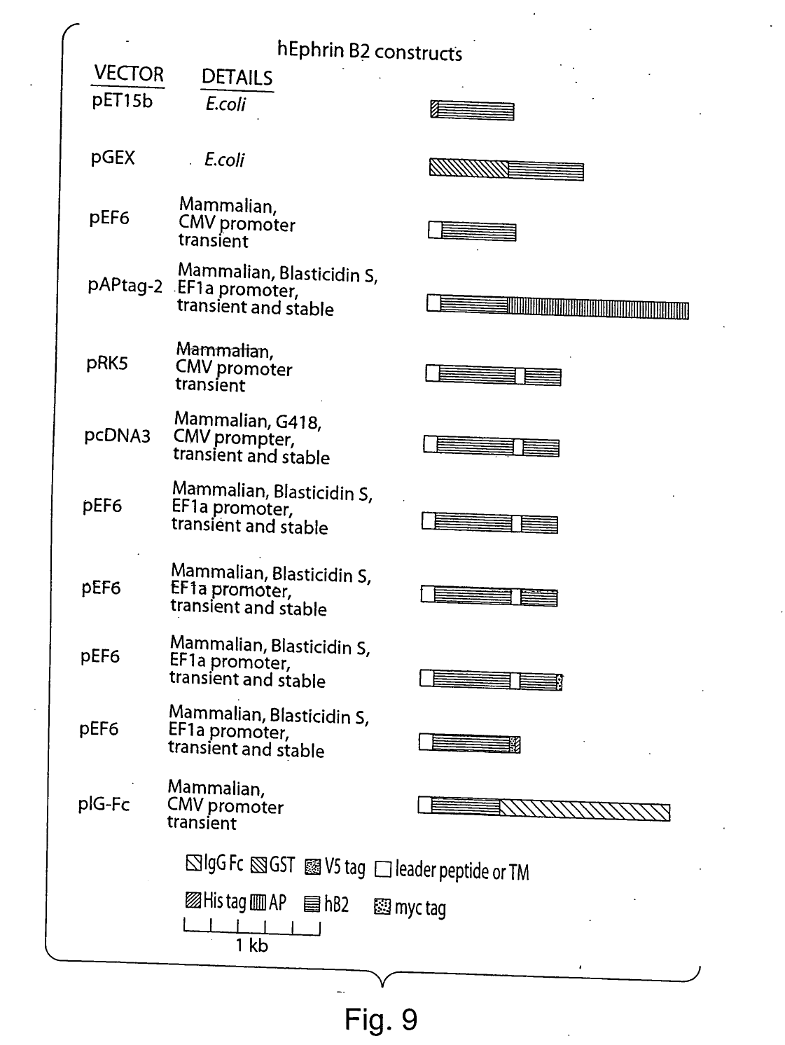

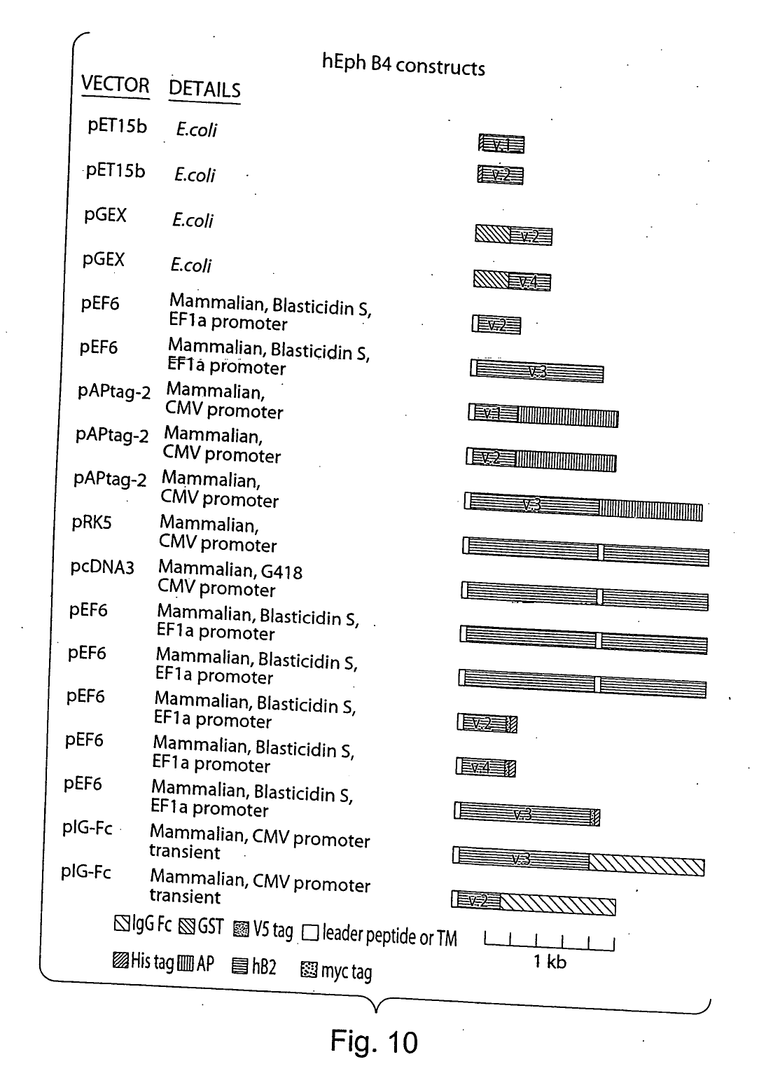

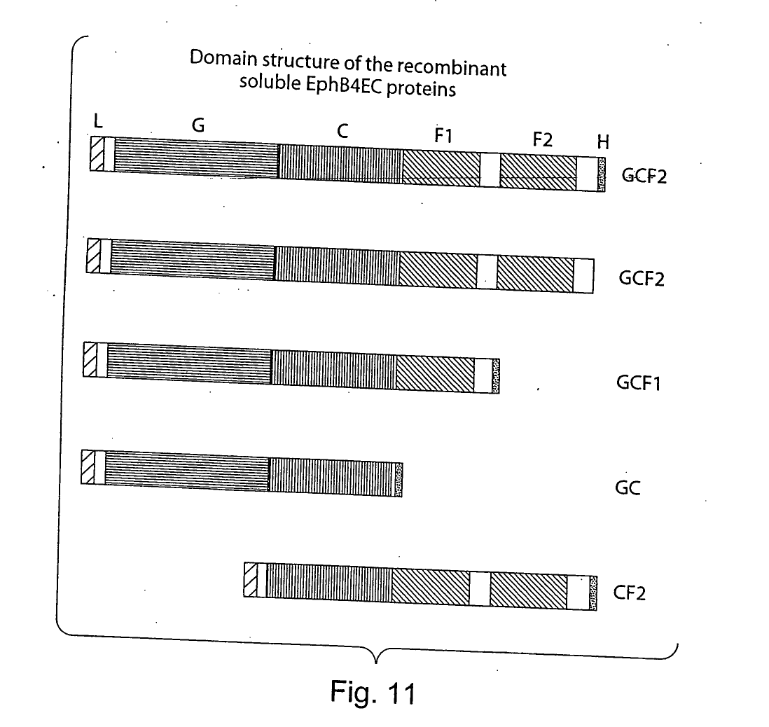

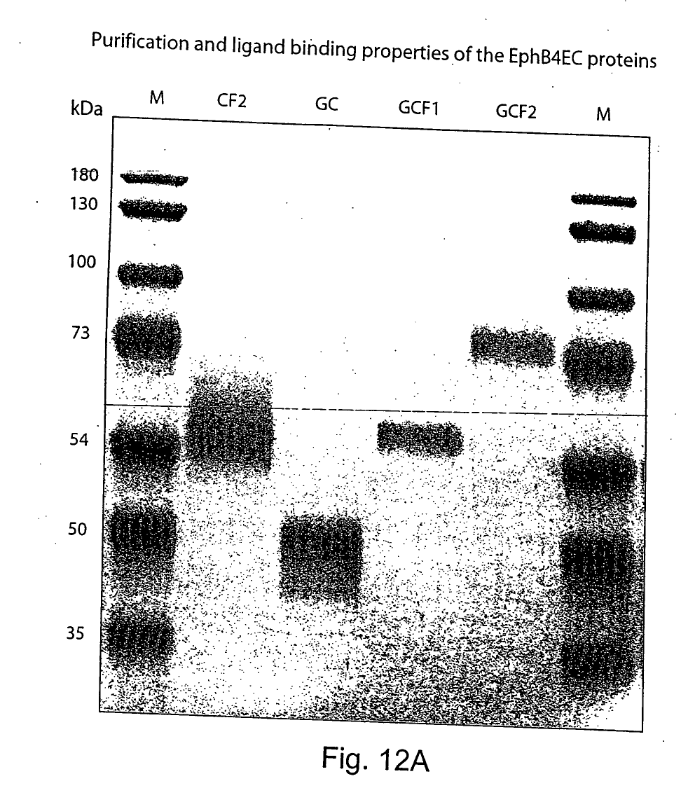

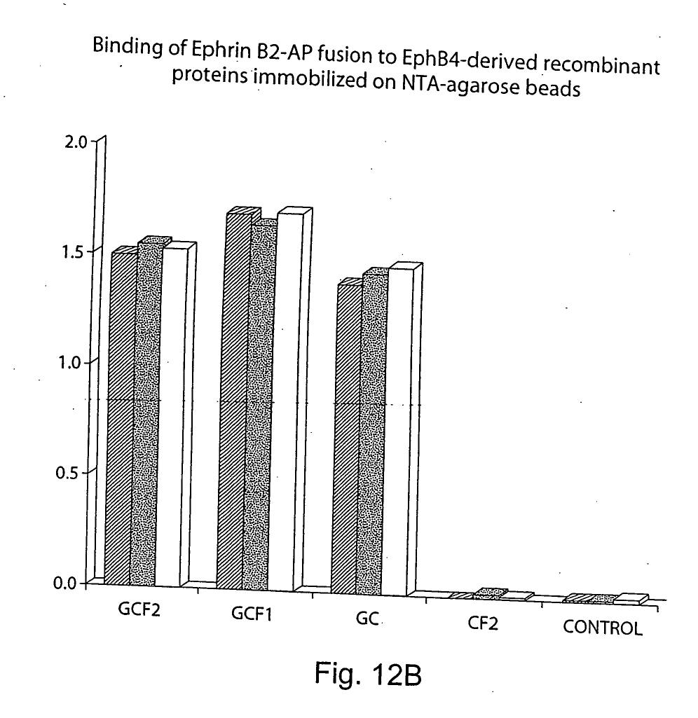

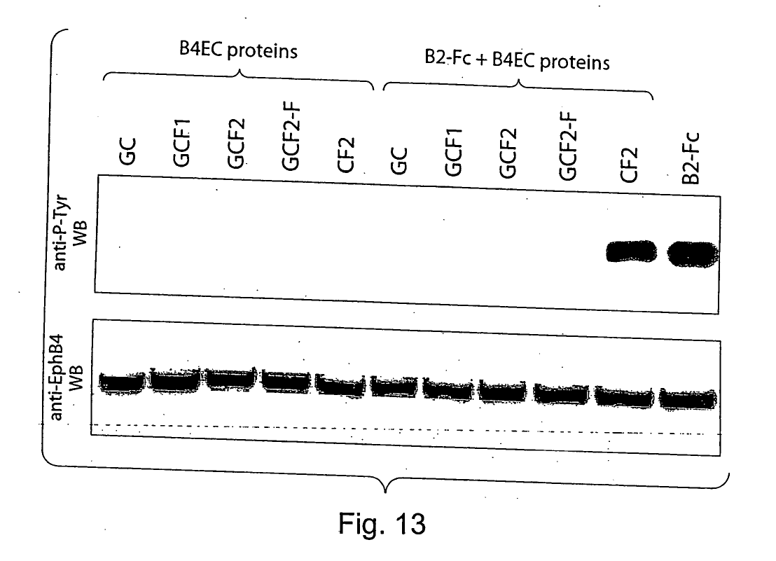

Soluble derivatives of the extracellular domains of human EphrinB2 and EphB4 proteins represent either truncated full-length predicted extracellular domains of EphrinB2 (B4ECv3, B2EC) or translational fusions of the domains with constant region of human immunoglobulins (IgG1 Fc fragment), such as B2EC-FC, B4ECv2-FC and B4ECv3-FC. Representative human EphrinB2 constructs and human EphB4 constructs are shown in FIGS. 9 and 10.

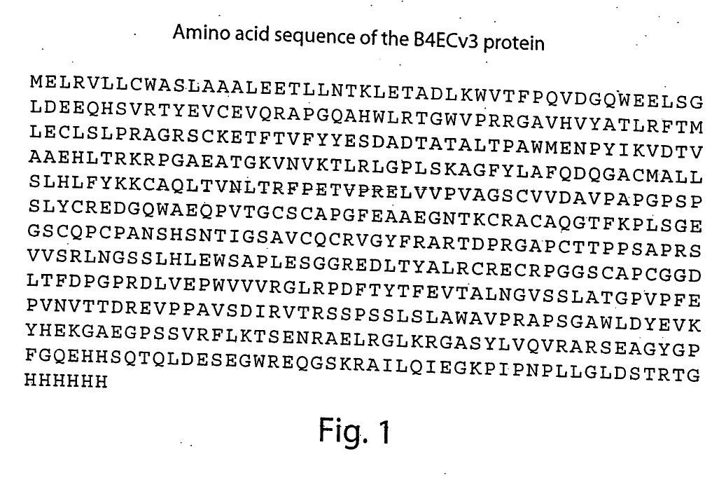

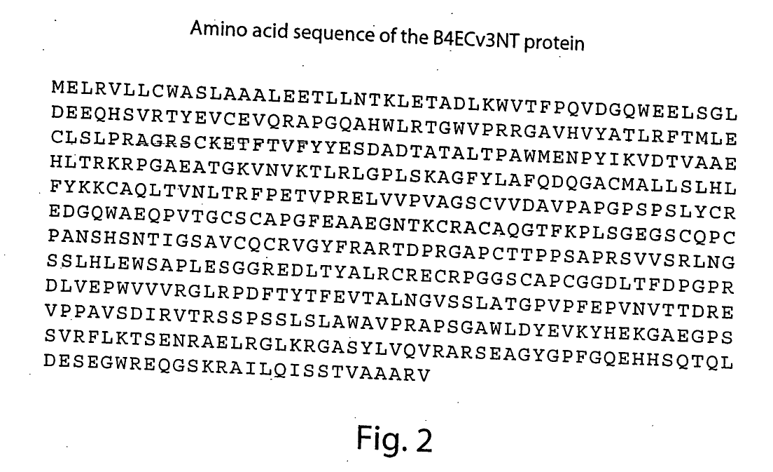

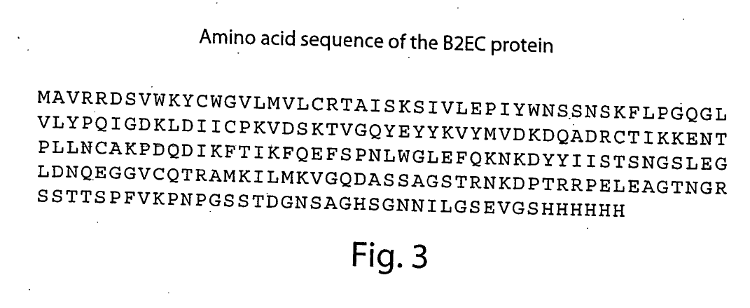

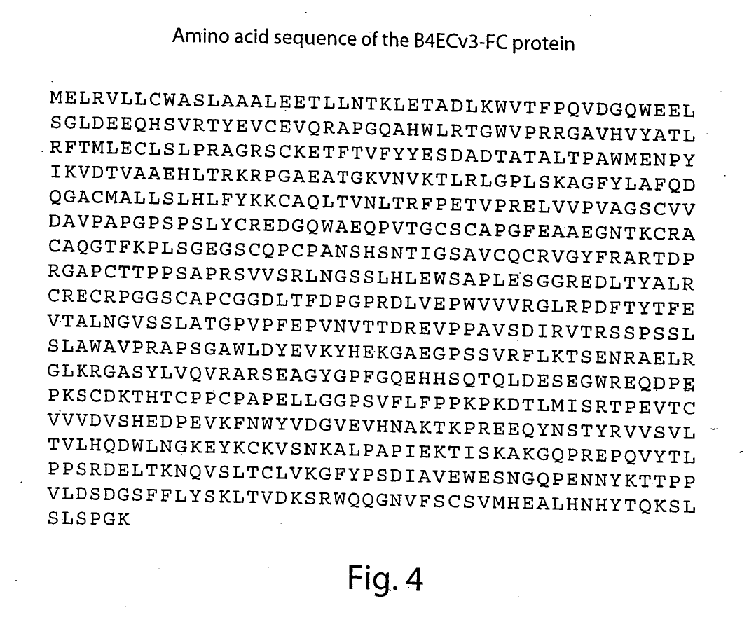

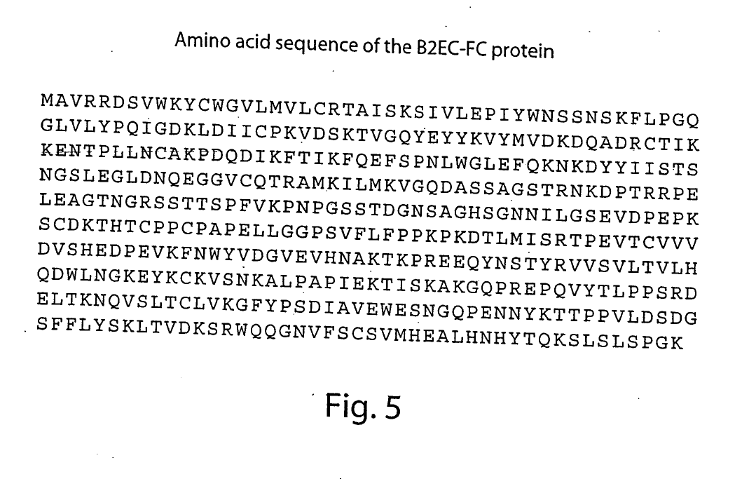

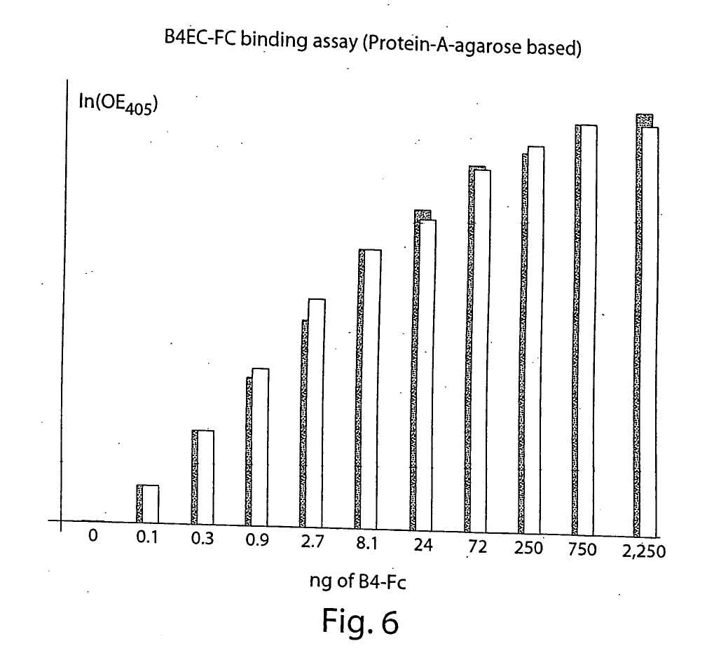

The cDNA fragments encoding these recombinant proteins were subcloned into mammalian expression vectors, expressed in transiently or stably transfected mammalian cell lines and purified to homogeneity as described in detail in Materials and Methods section (see below). Predicted amino acid sequences of the proteins are shown in FIGS. 1-5. High purity of the isolated proteins and their recognition by the corresponding anti-EphrinB2 and anti-EphB4 monoclonal or polyclonal antibodies were confirmed. The recombinant proteins exhibit the expected high-affinity binding, binding competition and specificity properties with their corresponding binding partners as corroborated by the biochemical assays (e.g., FIGS. 6-8).

Such soluble derivative proteins human EphrinB2 and EphB4 exhibit potent biological activity in several cell-based assays and in vivo assays which measure angiogenesis or anti-cancer activities, and are therefore effective in modulating the EphrinB2-EphB4 signaling axis in vivo.

The sequence of the Globular domain+Cys-rich domain (B4EC-GC, also called B4-GC), precursor protein is below (SEQ ID NO: 12):

MELRVLLCWASLAAALEETLLNTKLETADLKWVTFPQVDGQWEELSGLDE
EQHSVRTYEVCEVQRAPGQAHWLRTGWVPRRGAVHVYATLRFTMLECLSL
PRAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFEAAEGNTKCRACAQGTFKPLSGEGSCQPCPANSHSNTIGSA
VCQCRVGYFRARTDPRGAPCTTPPSAHHHHHH

For many uses including therapeutic use, the leader sequence (first 15 amino acids, so that the processed form begins Leu-Glu-Glu . . . ) and the c-terminal hexahistidine tag may be removed or omitted.

The sequence of the GCF precursor protein is below (SEQ ID NO: 13):

MELRVLLCWASLAAALEETLLNTKLETADLKWVTFPQVDGQWEELSGLDE
EQHSVRTYEVCEVQRAPGQAHWLRTGWVPRRGAVHVYATLRFTMLECLSL
PRAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFAEGNTKCRACAQGTFKPLSGEGSCQPCPANSHSNTIGSAVC
QCRVGYFRARTDPRGAPCTTPPSAPRSVVSRLNGSSLHLEWSAPLESGGR
EDLTYALRCRECRPGGSCAPCGGDLTFDPGPRDLVEPWVVVRGLRPDFTY
TFEVTALNGVSSLATGPVPFEPVNVHHHHHH

For many uses including therapeutic use, the leader sequence (first 15 amino acids, so that the processed form begins Leu-Glu-Glu . . . ) and the c-terminal hexahistidine tag may be removed or omitted.

The amino acid sequence of encoded FL-hB4EC precursor (His-tagged) is below (SEQ ID NO: 14):

MELRVLLCWASLAAALEETLLNTKLETADLKWVTFPQVDGQWEELSGLDE
EQHSVRTYEVCEVQPAPGQAHWLRTGWVPRRGAVHVYATLRFTMLECLSL
PRAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFEAAEGNTKCRACAQGTFKPLSGEGSCQPCPANSHSNTIGSA
VCQCRVGYFRARTDPRGAPCTTPPSAPRSVVSRLNGSSLHLEWSAPLESG
GREDLTYALRCRECRPGGSCAPCGGDLTFDPGPRDLVEPWVVVRGLRPDF
TYTFEVTALNGVSSLATGPVPFEPVNVTTDREVPPAVSDIRVTRSSPSSL
SLAWAVPPAPSGAWLDYEVKYHEKGAEGPSSVRFLKTSENRAELRGLKRG
ASYLVQVRARSEAGYGPFGQEHHSQTQLDESEGWREQGSKRAILQIEGKP
IPNPLLGLDSTRTGHHHHHH

For many uses including therapeutic use, the leader sequence (first 15 amino acids, so that the processed form begins Leu-Glu-Glu . . . ) and the c-terminal hexahistidine tag may be removed or omitted.

The sequence of EphB4 CF2 protein, precursor is below (SEQ ID NO: 15):

MELRVLLCWASLAAALEETLLNTKLETQLTVNLTRFPETVPRELVVPVAG
SCVVDAVPAPGPSPSLYCREDGQWAEQPVTGCSCAPGFEAAEGNTKCRAC
AQGTFKPLSGEGSCQPCPANSHSNTIGSAVCQCRVGYFRARTDPRGAPCT
TPPSAPRSVVSRLNGSSLHLEWSAPLESGGREDLTYALRCRECRPGGSCA
PCGGDLTFDPGPRDLVEPWVVVRGLRPDFTYTFEVTALNGVSSLATGPVP
FEPVNVTTDREVPPAVSDIRVTRSSPSSLSLAWAVPRAPSGAWLDYEVKY
HEKGAEGPSSVRFLKTSENRAELRGLKRGASYLVQVRARSEAGYGPFGQE
HHSQTQLDESEGWREQGGRSSLEGPRFEGKPIPNPLLGLDSTRTGHHHHH
H

The precursor sequence of the preferred GCF2 protein (also referred to herein as GCF2F, B4EC) is below (SEQ ID NO: 16):

MELRVLLCWASLAAALEETLLNTKLETADLKWVTFPQVDGQWEELSGLDE
EQHSVRTYEVCEVQRAPGQAHWLRTGWVPRRGAVHVYATLRFTMLECLSL
PRAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFEAAEGNTKCRACAQGTFKPLSGEGSCQPCPANSHSNTIGSA
VCQCRVGYFRARTDPRGAPCTTPPSAPRSVVSRLNGSSLHLEWSAPLESG
GREDLTYALRCRECRPGGSCAPCGGDLTFDPGPRDLVEPWVVVRGLRPDF
TYTFEVTALNGVSSLATGPVPFEPVNVTTDREVPPAVSDIRVTRSSPSSL
SLAWAVPRAPSGAWLDYEVKYHEKGAEGPSSVRFLKTSENRAELRGLKRG
ASYLVQVRARSEAGYGPFGQEHHSQTQLDESEGWREQ

The processed sequence is below (SEQ ID NO: 17):

LEETLLNTKLETADLKWVTFPQVDGQWEELSGLDEEQHSVRTYEVCEVQR
APGQAHWLRTGWVPRRGAVHVYATLRFTMLECLSLPRAGRSCKETFTVFY
YESDADTATALTPAWMENPYIKVDTVAAEHLTRKRPGAEATGKVNVKTLR
LGPLSKAGFYLAFQDQGACMALLSLHLFYKKCAQLTVNLTRFPETVPREL
VVPVAGSCVVDAVPAPGPSPSLYCREDGQWAEQPVTGCSCAPGFEAAEGN
TKCRACAQGTFKPLSGEGSCQPCPANSHSNTIGSAVCQCRVGYFRARTDP
RGAPCTTPPSAPRSVVSRLNGSSLHLEWSAPLESGGREDLTYALRCRECR
PGGSCAPCGGDLTFDPGPRDLVEPWVVVRGLRPDFTYTFEVTALNGVSSL
ATGPVPFEPVNVTTDREVPPAVSDIRVTRSSPSSLSLAWAVPRAPSGAWL
DYEVKYHEKGAEGPSSVRFLKTSENRAELRGLKRGASYLVQVRARSEAGY
GPFGQEHHSQTQLDESEGWREQ

Biochemical Assays

A. Binding Assay

10 μl of Ni-NTA-Agarose were incubated in microcentrifuge tubes with 5011 of indicated amount of B4ECv3 diluted in binding buffer BB (20 mM Tris-HCl, 0.15 M NaCl, 0.1% bovine serum albumin pH 8) After incubation for 30 min on shaking platform, Ni-NTA beads were washed twice with 1.4 ml of BB, followed by application of 50 μl of B2-AP in the final concentration of 50 nM. Binding was performed for 30 min on shaking platform, and then tubes were centrifuged and washed one time with 1.4 ml of BB. Amount of precipitated AP was measured colorimetrically after application of PNPP.

B. Inhibition Assay

Inhibition in solution. Different amounts of B4ECv3 diluted in 50 μl of BB were pre-incubated with 50 μl of 5 nM B2EC-AP reagent (protein fusion of EphrinB2 ectodomain with placental alkaline phosphatase). After incubation for 1 h, unbound B2EC-AP was precipitated with 5,000 HEK293 cells expressing membrane-associated full-length EphB4 for 20 min. Binding reaction was stopped by dilution with 1.2 ml of BB, followed by centrifugation for 10 min. Supernatants were discarded and alkaline phosphatase activities associated with collected cells were measured by adding para-nitrophenyl phosphate (PNPP) substrate.

C. B4EC-FC Binding Assay

Protein A-agarose based assay. 10 μl of Protein A-agarose were incubated in Eppendorf tubes with 5011 of indicated amount of B4EC-FC diluted in binding buffer BB (20 mM Tris-HCl, 0.15 M NaCl, 0.1% BSA pH 8). After incubation for 30 min on shaking platform, Protein A agarose beads were washed twice with 1.4 ml of BB, followed by application of 50 μl of B2ECAP reagent at the final concentration of 50 nM. Binding was performed for 30 min on shaking platform, and then tubes were centrifuged and washed once with 1.4 ml of BB. Colorimetric reaction of precipitated AP was measured after application of PNPP (FIG. 6).

Nitrocellulose based assay. B4EC-FC was serially diluted in 20 mM Tris-HCl, 0.15 M NaCl, 50 μg/ml BSA, pH 8. 2 μl of each fraction were applied onto nitrocellulose strip and spots were dried out for 3 min. Nitrocellulose strip was blocked with 5% non-fat milk for 30 min, followed by incubation with 5 nM B2EC-AP reagent. After 45 min incubation for binding, nitrocellulose washed twice with 20 mM Tris-HCl, 0.15 M NaCl, 50 μg/ml BSA, pH 8 and color was developed by application of alkaline phosphatase substrate Sigma Fast (Sigma).

D. B4EC-FC Inhibition Assay

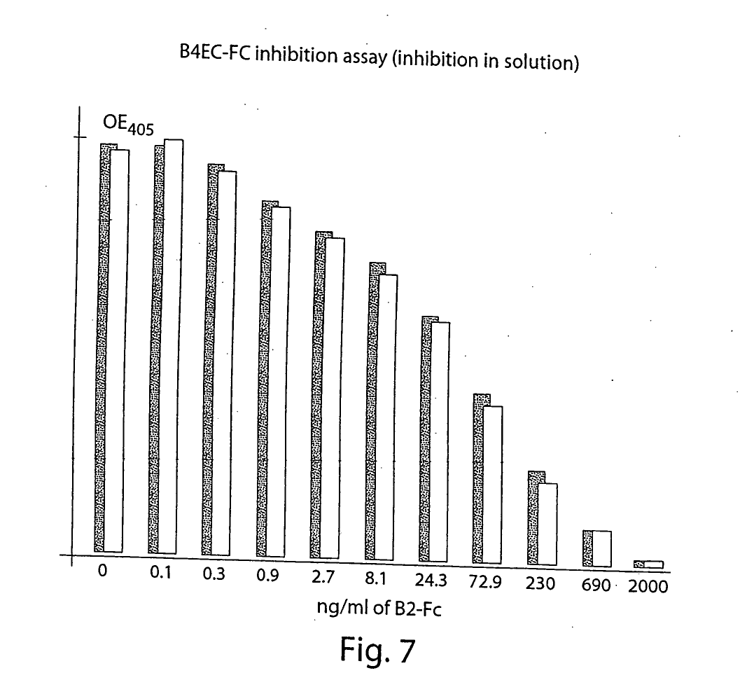

Inhibition in solution. See above, for B4ECv3. The results were shown in FIG. 7.

E. B2EC-FC Binding Assay

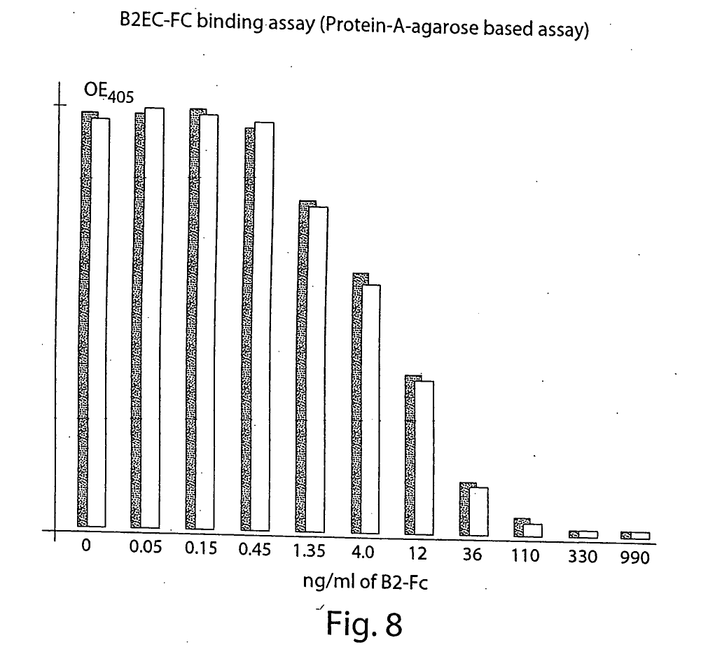

Protein-A-agarose based assay. See above, for B4EC-FC. The results were shown in FIG. 8.

Nitrocellulose based assay. See above, for B4EC-FC.

### Example 2

### Inhibition of EphrinB2 Gene Expression by EphrinB2 Antisense Probes and RNAi Probes

KS SLK, a cell line expressing endogenous high level of EphrinB2. Cell viability was tested using fixed dose of each oligonucleotide (5 μM). Gene expression downregulation was done using cell line 293 engineered to stably express full-length EphrinB2. KS SLK expressing EphrinB2 were also used to test the viability in response to RNAi probes tested at the fixed dose of 50 nM. Protein expression levels were measured using 293 cells stably expressing full-length EphrinB2, in cell lysates after 24 hr treatment with fixed 50 nM of RNAi probes.

The results on EphrinB2 antisense probes were summarized below in Table 1. The results on EphrinB2 RNAi probes were summarized below in Table 2.

TABLE 1
EphrinB2 antisense ODNs.

| | | | |
|---|---|---|---|
| | | Percent | Inhibition |
| | Coding | reduction in | of EphrinB2 |
| Sequence (SEQ ID NO.) | region | viability | Expression |

| | | | | |
|---|---|---|---|---|
| Ephrin AS-51 | TCA GAC CTT GTA GTA AAT GT | (983-1002) | 35 | ++ |
| | (SEQ ID NO.21) | | | |
| Ephrin AS-50 | TCG CCG GGC TCT GCG GGG GC | (963-982) | 50 | +++ |
| | (SEQ ID NO.22) | | | |
| Ephrin AS-49 | ATC TCC TGG ACG ATG TAC AC | (943-962) | 45 | ++ |
| | (SEQ ID NO.23) | | | |
| Ephrin AS-48 | CGG GTG CCC GTA GTC CCC GC | (923-942) | 35 | ++ |
| | (SEQ ID NO.24) | | | |
| Ephrin AS-47 | TGA CCT TCT CGT AGT GAG GG | (903-922) | 40 | +++ |
| | (SEQ ID NO.25) | | | |
| Ephrin AS-46 | CAG AAG ACG CTG TCC GCA GT | (883-902) | 40 | ++ |
| | (SEQ ID NO.26) | | | |
| Ephrin AS-45 | CCT TAG CGG GAT GAT AAT GT | (863-882) | 35 | ++ |
| | (SEQ ID NO.27) | | | |
| Ephrin AS-44 | CAC TGG GCT CTG AGC CGT TG | (843-862) | 60 | +++ |
| | (SEQ ID NO.28) | | | |
| Ephrin AS-43 | TTG TTG CCG CTG CGC TTG GG | (823-842) | 40 | ++ |
| | (SEQ ID NO.29) | | | |
| Ephrin AS-42 | TGT GGC CAG TGT GCT GAG CG | (803-822) | 40 | ++ |
| | (SEQ ID NO.30) | | | |
| Ephrin AS-41 | ACA GCG TGG TCG TGT GCT GC | (783-802) | 70 | +++ |
| | (SEQ ID NO.31) | | | |
| Ephrin AS-40 | GGC GAG TGC TTC CTG TGT CT | (763-782) | 80 | ++++ |
| | (SEQ ID NO.32) | | | |
| Ephrin AS-39 | CCT CCG GTA CTT CAG CAA GA | (743-762) | 50 | +++ |
| | (SEQ ID NO.33) | | | |
| Ephrin AS-38 | GGA CCA CCA GCG TGA TGA TG | (723-742) | 60 | +++ |
| | (SEQ ID NO.34) | | | |
| Ephrin AS-37 | ATG ACG ATG AAG ATG ATG CA | (703-722) | 70 | +++ |
| | (SEQ ID NO.35) | | | |
| Ephrin AS-36 | TCC TGA AGC AAT CCC TGC AA | (683-702) | 60 | +++ |
| | (SEQ ID NO.36) | | | |
| Ephrin AS-35 | ATA AGG CCA CTT CGG AAC CG | (663-682) | 45 | ++ |
| | (SEQ ID NO.37) | | | |
| Ephrin AS-34 | AGG ATG TTG TTC CCC GAA TG | (643-662) | 50 | +++ |
| | (SEQ ID NO.38) | | | |
| Ephrin AS-33 | TCC GGC GCT GTT GCC GTC TG | (623-642) | 75 | +++ |
| | (SEQ ID NO.39) | | | |
| Ephrin AS-32 | TGC TAG AAC CTG GAT TTG GT | (603-622) | 60 | +++ |
| | (SEQ ID NO.40) | | | |
| Ephrin AS-31 | TTT ACA AAG GGA CTT GTT GT | (583-602) | 66 | +++ |
| | (SEQ ID NO.41) | | | |
| Ephrin AS-30 | CGA ACT TCT TCC ATT TGT AC | (563-582) | 50 | ++ |
| | (SEQ ID NO.42) | | | |
| Ephrin AS-29 | CAG CTT CTA GTT CTG GAC GT | (543-562) | 50 | +++ |
| | (SEQ ID NO.43) | | | |
| Ephrin AS-28 | CTT GTT GGA TCT TTA TTC CT | (523-542) | 70 | +++ |
| | (SEQ ID NO.44) | | | |
| Ephrin AS-27 | GGT TGA TCC AGC AGA ACT TG | (503-522) | 65 | +++ |
| | (SEQ ID NO.45) | | | |
| Ephrin AS-26 | CAT CTT GTC CAA CTT TCA TG | (483-502) | 75 | +++ |
| | (SEQ ID NO.46) | | | |
| Ephrin AS-25 | AGG ATC TTC ATG GCT CTT GT | (463-482) | 60 | +++ |
| | (SEQ ID NO.47) | | | |
| Ephrin AS-24 | CTG GCA CAC CCC TCC CTC CT | (443-462) | 45 | ++ |
| | (SEQ ID NO.48) | | | |
| Ephrin AS-23 | GGT TAT CCA GGC CCT CCA AA | (423-442) | 50 | +++ |
| | (SEQ ID NO.49) | | | |
| Ephrin AS-22 | GAC CCA TTT GAT GTA GAT AT | (403-422) | 50 | +++ |
| | (SEQ ID NO.50) | | | |
| Ephrin AS-21 | AAT GTA ATA ATC TTT GTT CT | (383-402) | 60 | +++ |
| | (SEQ ID NO.51) | | | |
| Ephrin AS-20 | TCT GAA ATT CTA GAC CCC AG | (363-382) | 60 | +++ |
| | (SEQ ID NO.52) | | | |
| Ephrin AS-19 | AGG TTA GGG CTG AAT TCT TG | (343-362) | 75 | +++ |
| | (SEQ ID NO.53) | | | |
| Ephrin AS-18 | AAA CTT GAT GGT GAA TTT GA | (323-342) | 60 | +++ |
| | (SEQ ID NO.54) | | | |
| Ephrin AS-17 | TAT CTT GGT CTG GTT TGG CA | (303-322) | 50 | ++ |
| | (SEQ ID NO.55) | | | |
| Ephrin AS-16 | CAG TTG AGG AGA GGG GTA TT | (283-302) | 40 | ++ |
| | (SEQ ID NO.56) | | | |
| Ephrin AS-15 | TTC CTT CTT AAT AGT GCA TC | (263-282) | 66 | +++ |
| | (SEQ ID NO.57) | | | |
| Ephrin AS-14 | TGT CTG CTT GGT CTT TAT CA | (243-262) | 70 | ++++ |
| | (SEQ ID NO.58) | | | |
| Ephrin AS-13 | ACC ATA TAA ACT TTA TAA TA | (223-242) | 50 | +++ |
| | (SEQ ID NO.59) | | | |
| Ephrin AS-12 | TTC ATA CTG GCC AAC AGT TT | (203-222) | 50 | +++ |
| | (SEQ ID NO.60) | | | |
| Ephrin AS-11 | TAG AGT CCA CTT TGG GGC AA | (183-202) | 70 | ++++ |
| | (SEQ ID NO.61) | | | |
| Ephrin AS-10 | ATA ATA TCC AAT TTG TCT CC | (163-182) | 70 | ++++ |
| | (SEQ ID NO.62) | | | |
| Ephrin AS-9 | TAT CTG TGG GTA TAG TAC CA | (143-162) | 80 | ++++ |
| | (SEQ ID NO.63) | | | |
| Ephrin AS-8 | GTC CTT GTC CAG GTA GAA AT | (123-142) | 60 | +++ |
| | (SEQ ID NO.64) | | | |
| Ephrin AS-7 | TTG GAG TTC GAG GAA TTC CA | (103-122) | 80 | ++++ |
| | (SEQ ID NO.65) | | | |
| Ephrin AS-6 | ATA GAT AGG CTC TAA AAC TA | (83-102) | 70 | +++ |
| | (SEQ ID NO.66) | | | |
| Ephrin AS-5 | TCG ATT TGG AAA TCG CAG TT | (63-82) | 50 | +++ |
| | (SEQ ID NO.67) | | | |
| Ephrin AS-4 | CTG CAT AAA ACC ATC AAA AC | (43-62) | 80 | ++++ |
| | (SEQ ID NO.68) | | | |
| Ephrin AS-3 | ACC CCA GCA GTA CTT CCA CA | (23-42) | 85 | ++++ |
| | (SEQ ID NO.69) | | | |
| Ephrin AS-2 | CGG AGT CCC TTC TCA CAG CC | (3-22) | 70 | +++ |
| | (SEQ ID NO.70) | | | |
| Ephrin AS-1 | GAG TCC CTT CTC ACA GCC AT | (1-20) | 80 | ++++ |
| | (SEQ ID NO.71) | | | |

TABLE 2
EphrinB2 RNAi probes.

| | | | |
|---|---|---|---|
| RNAi Sequence (read “t” as | Percent | Inhibition | |
| “u”) and homology with | reduction in | of EphrinB2 | RNAi |
| other human genes. (SEQ ID NO:) | viability | Expression | no. |

| | | | | | |
|---|---|---|---|---|---|
| 89 | aactgcgatttccaaatcgat | 109 | 80 | ++++ | 1 |
| | (SEQ ID NO:72) | | | | |
| 141 | aactccaaatttctacctgga | 161 | 70 | ++++ | 2 |
| | (SEQ ID NO:73) | | | | |
| 148 | aatttctacctggacaaggac | 168 | 75 | +++ | 3 |
| | (SEQ ID NO:74) | | | | |
| 147 | aaatttctacctggacaagga | 167 | 60 | +++ | 4 |
| | (SEQ ID NO:75) | | | | |
| 163 | aaggactggtactatacccac | 183 | 40 | ++ | 5 |
| | (SEQ ID NO:76) | | | | |
| 217 | aagtggactctaaaactgttg | 237 | 80 | ++++ | 6 |
| | (SEQ ID NO:77) | | | | |
| 229 | aaactgttggccagtatgaat | 249 | 50 | +++ | 7 |
| | (SEQ ID NO:78) | | | | |
| 228 | aaaactgttggccagtatgaa | 248 | 80 | ++++ | 8 |
| | (SEQ ID NO:79) | | | | |
| 274 | aagaccaagcagacagatgca | 294 | 80 | ++++ | 11 |
| | (SEQ ID NO:80) | | | | |
| 273 | aaagaccaagcagacagatgc | 293 | 60 | +++ | 12 |
| | (SEQ ID NO:81) | | | | |
| 363 | aagtttcaagaattcagccct | 383 | 66 | +++ | 13 |
| | (SEQ ID NO:82) | | | | |
| 370 | aagaattcagccctaacctct | 390 | 50 | +++ | 14 |
| | (SEQ ID NO:83) | | | | |
| 373 | aattcagccctaacctctggg | 393 | 50 | +++ | 15 |
| | (SEQ ID NO:84) | | | | |
| 324 | aactgtgccaaaccagaccaa | 344 | 90 | ++++ | 16 |
| | (SEQ ID NO:85) | | | | |
| 440 | aaatgggtctttggagggcct | 460 | 80 | ++++ | 17 |
| | (SEQ ID NO:86) | | | | |
| 501 | aagatcctcatgaaagttgga | 521 | 50 | +++ | 18 |
| | (SEQ ID NO:87) | | | | |
| 513 | aaagttggacaagatgcaagt | 533 | 50 | +++ | 19 |
| | (SEQ ID NO:88) | | | | |
| 491 | aagagccatgaagatcctcat | 511 | 50 | +++ | 20 |
| | (SEQ ID NO:89) | | | | |
| 514 | aagttggacaagatgcaagtt | 534 | 66 | +++ | 21 |
| | (SEQ ID NO:90) | | | | |
| 523 | aagatgcaagttctgctggat | 543 | 66 | +++ | 22 |
| | (SEQ ID NO:91) | | | | |
| 530 | aagttctgctggatcaaccag | 550 | 50 | +++ | 23 |
| | (SEQ ID NO:92) | | | | |
| 545 | aaccaggaataaagatccaac | 565 | 35 | ++ | 24 |
| | (SEQ ID NO:93) | | | | |
| 555 | aaagatccaacaagacgtcca | 575 | 40 | ++ | 25 |
| | (SEQ ID NO:94) | | | | |
| 556 | aagatccaacaagacgtccag | 576 | 60 | +++ | 26 |
| | (SEQ ID NO:95) | | | | |
| 563 | aacaagacgtccagaactaga | 583 | 60 | +++ | 27 |
| | (SEQ ID NO:96) | | | | |
| 566 | aagacgtccagaactagaagc | 586 | 70 | +++ | 28 |
| | (SEQ ID NO:97) | | | | |
| 593 | aaatggaagaagttcgacaac | 613 | 75 | ++++ | 29 |
| | (SEQ ID NO:98) | | | | |
| 577 | aactagaagctggtacaaatg | 597 | 66 | +++ | 30 |
| | (SEQ ID NO:99) | | | | |
| 594 | aatggaagaagttcgacaaca | 614 | 35 | ++ | 31 |
| | (SEQ ID NO:100) | | | | |
| 583 | aagctggtacaaatggaagaa | 603 | 50 | +++ | 32 |
| | (SEQ ID NO:101) | | | | |
| 611 | aacaagtccctttgtaaaacc | 631 | 70 | ++++ | 33 |
| | (SEQ ID NO:102) | | | | |
| 599 | aagaagttcgacaacaagtcc | 619 | 70 | ++++ | 34 |
| | (SEQ ID NO:103) | | | | |
| 602 | aagttcgacaacaagtccctt | 622 | 80 | ++++ | 35 |
| | (SEQ ID NO:104) | | | | |
| 626 | aaaaccaaatccaggttctag | 646 | 50 | +++ | 36 |
| | (SEQ ID NO:105) | | | | |
| 627 | aaaccaaatccaggttctagc | 647 | 25 | + | 37 |
| | (SEQ ID NO:106) | | | | |
| 628 | aaccaaatccaggttctagca | 648 | 30 | ++ | 38 |
| | (SEQ ID NO:107) | | | | |
| 632 | aaatccaggttctagcacaga | 652 | 60 | +++ | 39 |
| | (SEQ ID NO:108) | | | | |
| 633 | aatccaggttctagcacagac | 653 | 40 | ++ | 40 |
| | (SEQ ID NO:109) | | | | |
| 678 | aacaacatcctcggttccgaa | 698 | 30 | ++ | 41 |
| | (SEQ ID NO:110) | | | | |
| 681 | aacatcctcggttccgaagtg | 701 | 20 | + | 42 |
| | (SEQ ID NO:111) | | | | |
| 697 | aagtggccttatttgcaggga | 717 | 30 | ++ | 43 |
| | (SEQ ID NO:112) | | | | |

Additional EphrinB2 RNAi probes described in the
specification

| | |
|---|---|
| GCAGACAGAUGCACUAUUAUU | ephrin |
| (SEQ ID NO:113) | B2 264 |
| CUGCGAUUUCCAAAUCGAUUU | ephrin |
| (SEQ ID NO:114) | B2 63 |
| GGACUGGUACUAUACCCACUU | ephrin |
| (SEQ ID NO:115) | B2 137 |

### Example 3

### HSA-EphB4 Ectodomain Fusion and PEG-Modified EphB4 Ectodomain

A. Generation of HSA-EphB4 Ectodomain Fusion

Human serum albumin fragment in XbaI-NotI form was PCR-amplified out for creating a fusion with GCF2, and TA-cloned into pEF6. In the next step, the resulting vector was cut with Xba I (partial digestion) and the HSA fragment (1.8 kb) was cloned into Xba I site of pEF6-GCF2-Xba to create fusion expression vector. The resulting vector had a point mutation C to T leading to Thr to Ile substitution in position 4 of the mature protein. It was called pEF6-GCF2-HSAmut. In the next cloning step, the mutation was removed by substituting wild type KpnI fragment from pEF6-GCF2-IF (containing piece of the vector and N-terminal part of GCF2) for the mutated one, this final vector was called pEF6-GCF2. The DNA sequence of pEF6-GCF2 was confirmed.

The predicted sequence of the HSA-EphB4 precursor protein was below (SEQ ID NO: 18):

MELRVLLCWASLAAALEETLLNTKLETADLKWVTFPQVDGQWEELSGLDE
EQHSVRTYEVCDVQRAPGQAHWLRTGWVPRRGAVHVYATLRFTMLECLSL
PRAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFEAAEGNTKCRACAQGTFKPLSGEGSCQPCPANSHSNTIGSA
VCQCRVGYFRARTDPRGAPCTTPPSAPRSVVSRLNGSSLHLEWSAPLESG
GREDLTYALRCRECRPGGSCAPCGGDLTFDPGPRDLVEPWVVVRGLRPDF
TYTFEVTALNGVSSLATGPVPFEPVNVTTDREVPPAVSDIRVTRSSPSSL
SLAWAVPPAPSGAVLDYEVKYHEKGAEGPSSVRFLKTSENRAELRGLKRG
ASYLVQVRARSEAGYGPFGQEHHSQTQLDESEGWREQSRDAHKSEVAHRF
KDLGEENFKALVLIAFAQYLQQCPFEDHVKLVNEVTEFAKTCVADESAEN
CDKSLHTLFGDKLCTVATLRETYGEMADCCAKQEPERNECFLQHKDDNPN
LPRLVRPEVDVMCTAFHDNEETFLKKYLYEIARRHPYFYAPELLFFAKRY
KAAFTECCQAADKAACLLPKLDELRDEGKASSAKQRLKCASLQKFGERAF
KAWAVARLSQRFPKAEFAEVSKLVTDLTKVHTECCHGDLLECADDRADLA
KYICENQDSISSKLKECCEKPLLEKSHCIAEVENDEMPADLPSLAADFVE
SKDVCKNYAEAKDVFLGMFLYEYARRHPDYSVVLLLRLAKTYETTLEKCC
AAADPHECYAKVFDEFKPLVEEPQNLIKQNCELFKQLGEYKFQNALLVRY
TKKVPQVSTPTLVEVSRNLGKVGSKCCKHPEAKRMPCAEDYLSVVLNQLC
VLHEKTPVSDRVTKCCTESLVNRRPCFSALEVDETYVPKEFNAETFTFHA
DICTLSEKERQIKKQTALVELVKHKPKATKEQLKAVMDDFAAFVEKCCKA
DDKETCFAEEGKKLVAASQAALGL

The predicted sequence of the mature form of the HSA-EphB4 protein was as follows (SEQ ID NO: 19):

LEETLLNTKLETADLKWVTFPQVDGQWEELSGLDEEQHSVRTYEVCDVQR
APGQAHWLRTGWVPRRGAVHVYATLRFTMLECLSLPRAGRSCKETFTVFY
YESDADTATALTPAWMENPYIKVDTVAAEHLTRKRPGAEATGKVNVKTLR
LGPLSKAGFYLAFQDQGACMALLSLHLFYKKCAQLTVNLTRFPETVPREL
VVPVAGSCVVDAVPAPGPSPSLYCREDGQWAEQPVTGCSCAPGFEAAEGN
TKCPACAQGTFKPLSGEGSCQPCPANSHSNTIGSAVCQCRVGYFRARTDP
RGAPCTTPPSAPRSVVSRLNGSSLHLEWSAPLESGGREDLTYALRCRECR
PGGSCAPCGGDLTFDPGPRDLVEPWVVVRGLRPDFTYTFEVTALNGVSSL
ATGPVPFEPVNVTTDREVPPAVSDIRVTRSSPSSLSLAWAVPRAPSGAVL
DYEVKYHEKGAEGPSSVRFLKTSENRAELRGLKRGASYLVQVRARSEAGY
GPFGQEHHSQTQLDESEGWREQSRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDPADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFKQLGEYKFQNALLVRYTKKVPQVSTPTLVEV
SRNLGKVGSKCCKHPEAKRNPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL

The nucleic acid sequence of the pEF6-GCF2 plasmid was below (SEQ ID NO: 20):

aatattattgaagcatttatcagggttattgtctcatgagcggatacata
tttgaatgtatttagaaaaataaacaaataggggttccgcgcacatttcc
ccgaaaagtgccacctgacgtcgacggatcgggagatctcccgatcccct
atggtcgactctcagtacaatctgctctgatgccgcatagttaagccagt
atctgctccctgcttgtgtgttggaggtcgctgagtagtgcgcgagcaaa
atttaagctacaacaaggcaaggcttgaccgacaattgcatgaagaatct
gcttagggttaggcgttttgcgctgcttcgcgatgtacgggccagatata
cgcgttgacattgattattgactaggcttttgcaaaaagctttgcaaaga
tggataaagttttaaacagagaggaatctttgcagctaatggaccttcta
ggtcttgaaaggagtgcctcgtgaggctccggtgcccgtcagtgggcaga
gcgcacatcgcccacagtccccgagaagttggggggaggggtcggcaatt
gaaccggtgcctagagaaggtggcgcggggtaaactgggaaagtgatgtc
gtgtactggctccgcctttttcccgagggtgggggagaaccgtatataag
tgcagtagtcgccgtgaacgttctttttcgcaacgggtttgccgccagaa
cacaggtaagtgccgtgtgtggttcccgcgggcctggcctctttacgggt
tatggcccttgcgtgccttgaattacttccacctggctgcagtacgtgat
tcttgatcccgagcttcgggttggaagtgggtgggagagttcgaggcctt
gcgcttaaggagccccttcgcctcgtgcttgagttgaggcctggcctggg
cgctggggccgccgcgtgcgaatctggtggcaccttcgcgcctgtctcgc
tgctttcgataagtctctagccatttaaaatttttgatgacctgctgcga
cgctttttttctggcaagatagtcttgtaaatgcgggccaagatctgcac
actggtatttcggtttttggggccgcgggcggcgacggggcccgtgcgtc
ccagcgcacatgttcggcgaggcggggcctgcgagcgcggccaccgagaa
tcggacgggggtagtctcaagctggccggcctgctctggtgcctggcctc
gcgccgccgtgtatcgccccgccctgggcggcaaggctggcccggtcggc
accagttgcgtgagcggaaagatggccgcttcccggccctgctgcaggga
gctcaaaatggaggacgcggcgctcgggagagcgggcgggtgagtcaccc
acacaaaggaaaagggcctttccgtcctcagccgtcgcttcatgtgactc
cacggagtaccgggcgccgtccaggcacctcgattagttctcgagctttt
ggagtacgtcgtctttaggttggggggaggggttttatgcgatggagttt
ccccacactgagtgggtggagactgaagttaggccagcttggcacttgat
gtaattctccttggaatttgccctttttgagtttggatcttggttcattc
tcaagcctcagacagtggttcaaagtttttttcttccatttcaggtgtcg
tgaggaattagcttggtactaatacgactcactatagggagacccaagct
ggctaggtaagcttggtaccgagctcggatccactagtccagtgtggtgg
aattgcccttCAAGCTTGCCGCCACCATGGAGCTCCGGGTGCTGCTCTGC
TGGGCTTCGTTGGCCGCAGCTTTGGAAGAGACCCTGCTGAACACAAAATT
GGAAACTGCTGATCTGAAGTGGGTGACATTCCCTCAGGTGGACGGGCAGT
GGGAGGAACTGAGCGGCCTGGATGAGGAACAGCACAGCGTGCGCACCTAC
GAAGTGTGTGACGTGCAGCGTGCCCCGGGCCAGGCCCACTGGCTTCGCAC
AGGTTGGGTCCCACGGCGGGGCGCCGTCCACGTGTACGCCACGCTGCGCT
TCACCATGCTCGAGTGCCTGTCCCTGCCTCGGGCTGGGCGCTCCTGCAAG
GAGACCTTCACCGTCTTCTACTATGAGAGCGATGCGGACACGGCCACGGC
CCTCACGCCAGCCTGGATGGAGAACCCCTACATCAAGGTGGACACGGTGG
CCGCGGAGCATCTCACCCGGAAGCGCCCTGGGGCCGAGGCCACCGGGAAG
GTGAATGTCAAGACGCTGCGCCTGGGACCGCTCAGCAAGGCTGGCTTCTA
CCTGGCCTTCCAGGACCAGGGTGCCTGCATGGCCCTGCTATCCCTGCACC
TCTTCTACAAAAAGTGCGCCCAGCTGACTGTGAACCTGACTCGATTCCCG
GAGACTGTGCCTCGGGAGCTGGTTGTGCCCGTGGCCGGTAGCTGCGTGGT
GGATGCCGTCCCCGCCCCTGGCCCCAGCCCCAGCCTCTACTGCCGTGAGG
ATGGCCAGTGGGCCGAACAGCCGGTCACGGGCTGCAGCTGTGCTCCGGGG
TTCGAGGCAGCTGAGGGGAACACCAAGTGCCGAGCCTGTGCCCAGGGCAC
CTTCAAGCCCCTGTCAGGAGAAGGGTCCTGCCAGCCATGCCCAGCCAATA
GCCACTCTAACACCATTGGATCAGCCGTCTGCCAGTGCCGCGTCGGGTAC
TTCCGGGCACGCACAGACCCCCGGGGTGCACCCTGCACCACCCCTCCTTC
GGCTCCGCGGAGCGTGGTTTCCCGCCTGAACGGCTCCTCCCTGCACCTGG
AATGGAGTGCCCCCCTGGAGTCTGGTGGCCGAGAGGACCTCACCTACGCC
CTCCGCTGCCGGGAGTGTCGACCCGGAGGCTCCTGTGCGCCCTGCGGGGG
AGACCTGACTTTTGACCCCGGCCCCCGGGACCTGGTGGAGCCCTGGGTGG
TGGTTCGAGGGCTACGTCCTGACTTCACCTATACCTTTGAGGTCACTGCA
TTGAACGGGGTATCCTCCTTAGCCACGGGGCCCGTCCCATTTGAGCCTGT
CAATGTCACCACTGACCGAGAGGTACCTCCTGCAGTGTCTGACATCCGGG
TGACGCGGTCCTCACCCAGCAGCTTGAGCCTGGCCTGGGCTGTTCCCCGG
GCACCCAGTGGGGCTGTGCTGGACTACGAGGTCAAATACCATGAGAAGGG
CGCCGAGGGTCCCAGCAGCGTGCGGTTCCTGAAGACGTCAGAAAACCGGG
CAGAGCTGCGGGGGCTGAAGCGGGGAGCCAGCTACCTGGTGCAGGTACGG
GCGCGCTCTGAGGCCGGCTACGGGCCCTTCGGCCAGGAACATCACAGCCA
GACCCAACTGGATGAGAGCGAGGGCTGGCGGGAGCAGtctagaGATGCAC
ACAAGAGTGAGGTTGCTCATCGGTTTAAAGATTTGGGAGAAGAAAATTTC
AAAGCCTTGGTGTTGATTGCCTTTGCTCAGTATCTTCAGCAGTGTCCATT
TGAAGATCATGTAAAATTAGTGAATGAAGTAACTGAATTTGCAAAAACAT
GTGTAGCTGATGAGTCAGCTGAAAATTGTGACAAATCACTTCATACCCTT
TTTGGAGACAAATTATGCACAGTTGCAACTCTTCGTGAAACCTATGGTGA
AATGGCTGACTGCTGTGCAAAACAAGAACCTGAGAGAAATGAATGCTTCT
TGCAACACAAAGATGACAACCCAAACCTCCCCCGATTGGTGAGACCAGAG
GTTGATGTGATGTGCACTGCTTTTCATGACAATGAAGAGACATTTTTGAA
AAAATACTTATATGAAATTGCCAGAAGACATCCTTACTTTTATGCCCCGG
AACTCCTTTTCTTTGCTAAAAGGTATAAAGCTGCTTTTACAGAATGTTGC
CAAGCTGCTGATAAAGCTGCCTGCCTGTTGCCAAAGCTCGATGAACTTCG
GGATGAAGGGAAGGCTTCGTCTGCCAAACAGAGACTCAAATGTGCCAGTC
TCCAAAAATTTGGAGAAAGAGCTTTCAAAGCATGGGCAGTGGCTCGCCTG
AGCCAGAGATTTCCCAAAGCTGAGTTTGCAGAAGTTTCCAAGTTAGTGAC
AGATCTTACCAAAGTCCACACGGAATGCTGCCATGGAGATCTGCTTGAAT
GTGCTGATGACAGGGCGGACCTTGCCAAGTATATCTGTGAAAATCAGGAT
TCGATCTCCAGTAAACTGAAGGAATGCTGTGAAAAACCTCTGTTGGAAAA
ATCCCACTGCATTGCCGAAGTGGAAAATGATGAGATGCCTGCTGACTTGC
CTTCATTAGCTGCTGATTTTGTTGAAAGTAAGGATGTTTGCAAAAACTAT
GCTGAGGCAAAGGATGTCTTCCTGGGCATGTTTTTGTATGAATATGCAAG
AAGGCATCCTGATTACTCTGTCGTGCTGCTGCTGAGACTTGCCAAGACAT
ATGAAACCACTCTAGAGAAGTGCTGTGCCGCTGCAGATCCTCATGAATGC
TATGCCAAAGTGTTCGATGAATTTAAACCTCTTGTGGAAGAGCCTCAGAA
TTTAATCAAACAAAACTGTGAGCTTTTTAAGCAGCTTGGAGAGTACAAAT
TCCAGAATGCGCTATTAGTTCGTTACACCAAGAAAGTACCCCAAGTGTCA
ACTCCAACTCTTGTAGAGGTCTCAAGAAACCTAGGAAAAGTGGGCAGCAA
ATGTTGTAAACATCCTGAAGCAAAAAGAATGCCCTGTGCAGAAGACTATC
TATCCGTGGTCCTGAACCAGTTATGTGTGTTGCATGAGAAAACGCCAGTA
AGTGACAGAGTCACAAAATGCTGCACAGAGTCCTTGGTGAACAGGCGACC
ATGCTTTTCAGCTCTGGAAGTCGATGAAACATACGTTCCCAAAGAGTTTA
ATGCTGAAACATTCACCTTCCATGCAGATATATGCACACTTTCTGAGAAG
GAGAGACAAATCAAGAAACAAACTGCACTTGTTGAGCTTGTGAAACACAA
GCCCAAGGCAACAAAAGAGCAACTGAAAGCTGTTATGGATGATTTCGCAG
CTTTTGTAGAGAAGTGCTGCAAGGCTGACGATAAGGAGACCTGCTTTGCC
GAGGAGGGTAAAAAACTTGTTGCTGCAAGTCAAGCTGCCTTAGGCTTATA
Atagcggccgcttaagggcaattctgcagatatccagcacagtggcggcc
gctcgagtctagagggcccgcggttcgaaggtaagcctatccctaaccct
ctcctcggtctcgattctacgcgtaccggtcatcatcaccatcaccattg
agtttaaacccgctgatcagcctcgactgtgccttctagttgccagccat
ctgttgtttgcccctcccccgtgccttccttgaccctggaaggtgccact
cccactgtcctttcctaataaaatgaggaaattgcatcgcattgtctgag
taggtgtcattctattctggggggtggggtggggcaggacagcaaggggg
aggattgggaagacaatagcaggcatgctggggatgcggtgggctctatg
gcttctgaggcggaaagaaccagctggggctctagggggtatccccacgc
gccctgtagcggcgcattaagcgcggcgggtgtggtggttacgcgcagcg
tgaccgctacacttgccagcgccctagcgcccgctcctttcgctttcttc
ccttcctttctcgccacgttcgccggctttccccgtcaagctctaaatcg
gggcatccctttagggttccgatttagtgctttacggcacctcgacccca
aaaaacttgattagggtgatggttcacgtagtgggccatcgccctgatag
acggtttttcgccctttgacgttggagtccacgttctttaatagtggact
cttgttccaaactggaacaacactcaaccctatctcggtctattcttttg
atttataagggattttggggatttcggcctattggttaaaaaatgagctg
atttaacaaaaatttaacgcgaattaattctgtggaatgtgtgtcagtta
gggtgtggaaagtccccaggctccccaggcaggcagaagtatgcaaagca
tgcatctcaattagtcagcaaccaggtgtggaaagtccccaggctcccca
gcaggcagaagtatgcaaagcatgcatctcaattagtcagcaaccatagt
cccgcccctaactccgcccatcccgcccctaactccgcccagttccgccc
attctccgccccatggctgactaattttttttatttatgcagaggccgag
gccgcctctgcctctgagctattccagaagtagtgaggaggcttttttgg
aggcctaggcttttgcaaaaagctcccgggagcttgtatatccattttcg
gatctgatcagcacgtgttgacaattaatcatcggcatagtatatcggca
tagtataatacgacaaggtgaggaactaaaccatggccaagcctttgtct
caagaagaatccaccctcattgaaagagcaacggctacaatcaacagcat
ccccatctctgaagactacagcgtcgccagcgcagctctctctagcgacg
gccgcatcttcactggtgtcaatgtatatcattttactgggggaccttgt
gcagaactcgtggtgctgggcactgctgctgctgcggcagctggcaacct
gacttgtatcgtcgcgatcggaaatgagaacaggggcatcttgagcccct
gcggacggtgtcgacaggtgcttctcgatctgcatcctgggatcaaagcg
atagtgaaggacagtgatggacagccgacggcagttgggattcgtgaatt
gctgccctctggttatgtgtgggagggctaagcacttcgtggccgaggag
caggactgacacgtgctacgagatttcgattccaccgccgccttctatga
aaggttgggcttcggaatcgttttccgggacgccggctggatgatcctcc
agcgcggggatctcatgctggagttcttcgcccaccccaacttgtttatt
gcagcttataatggttacaaataaagcaatagcatcacaaatttcacaaa
taaagcatttttttcactgcattctagttgtggtttgtccaaactcatca
atgtatcttatcatgtctgtataccgtcgacctctagctagagcttggcg
taatcatggtcatagctgtttcctgtgtgaaattgttatccgctcacaat
tccacacaacatacgagccggaagcataaagtgtaaagcctggggtgcct
aatgagtgagctaactcacattaattgcgttgcgctcactgcccgctttc
cagtcgggaaacctgtcgtgccagctgcattaatgaatcggccaacgcgc
ggggagaggcggtttgcgtattgggcgctcttccgcttcctcgctcactg
actcgctgcgctcggtcgttcggctgcggcgagcggtatcagctcactca
aaggcggtaatacggttatccacagaatcaggggataacgcaggaaagaa
catgtgagcaaaaggccagcaaaaggccaggaaccgtaaaaaggccgcgt
tgctggcgtttttccataggctccgcccccctgacgagcatcacaaaaat
cgacgctcaagtcagaggtggcgaaacccgacaggactataaagatacca
ggcgtttccccctggaagctccctcgtgcgctctcctgttccgaccctgc
cgcttaccggatacctgtccgcctttctcccttcgggaagcgtggcgctt
tctcaatgctcacgctgtaggtatctcagttcggtgtaggtcgttcgctc
caagctgggctgtgtgcacgaaccccccgttcagcccgaccgctgcgcct
tatccggtaactatcgtcttgagtccaacccggtaagacacgacttatcg
ccactggcagcagccactggtaacaggattagcagagcgaggtatgtagg
cggtgctacagagttcttgaagtggtggcctaactacggctacactagaa
ggacagtatttggtatctgcgctctgctgaagccagttaccttcggaaaa
agagttggtagctcttgatccggcaaacaaaccaccgctggtagcggtgg
tttttttgtttgcaagcagcagattacgcgcagaaaaaaaggatctcaag
aagatcctttgatcttttctacggggtctgacgctcagtggaacgaaaac
tcacgttaagggattttggtcatgagattatcaaaaaggatcttcaccta
gatccttttaaattaaaaatgaagttttaaatcaatctaaagtatatatg
agtaaacttggtctgacagttaccaatgcttaatcagtgaggcacctatc
tcagcgatctgtctatttcgttcatccatagttgcctgactccccgtcgt
gtagataactacgatacgggagggcttaccatctggccccagtgctgcaa
tgataccgcgagacccacgctcaccggctccagatttatcagcaataaac
cagccagccggaagggccgagcgcagaagtggtcctgcaactttatccgc
ctccatccagtctattaattgttgccgggaagctagagtaagtagttcgc
cagttaatagtttgcgcaacgttgttgccattgctacaggcatcgtggtg
tcacgctcgtcgtttggtatggcttcattcagctccggttcccaacgatc
aaggcgagttacatgatcccccatgttgtgcaaaaaagcggttagctcct
tcggtcctccgatcgttgtcagaagtaagttggccgcagtgttatcactc
atggttatggcagcactgcataattctcttactgtcatgccatccgtaag
atgcttttctgtgactggtgagtactcaaccaagtcattctgagaatagt
gtatgcggcgaccgagttgctcttgcccggcgtcaatacgggataatacc
gcgccacatagcagaactttaaaagtgctcatcattggaaaacgttcttc
ggggcgaaaactctcaaggatcttaccgctgttgagatccagttcgatgt
aacccactcgtgcacccaactgatcttcagcatcttttactttcaccagc
gtttctgggtgagcaaaaacaggaaggcaaaatgccgcaaaaaagggaat
aagggcgacacggaaatgttgaatactcatactcttcctttttc

B. Cell Culture and Transfections:

The human embryonic kidney cell line, 293T cells, was maintained in DMEM with 10% dialyzed fetal calf serum and 1% penicillin/streptomycin/neomycin antibiotics. Cells were maintained at 37° C. in a humidified atmosphere of 5% CO₂/95% air.

Transfections of plasmids encoding EphB4 ectodomain, fragments thereof, and EphB4-HSA fusions were performed using Lipofectamine 2000 reagent (Invitrogen) according to suggested protocol. One day before transfections, 293T cells were seeded at a high density to reach 80% confluence at the time of transfection. Plasmid DNA and Lipofectamine reagent at 1:3 ratio were diluted in Opti-MEM I reduced serum medium (Invitrogen) for 5 min and mixed together to form DNA-Lipofectamine complex. For each 10 cm culture dish, 10 μg of plasmid DNA was used. After 20 min, the above complex was added directly to cells in culture medium. After 16 hours of transfection, medium was aspirated, washed once with serum free DMEM and replaced with serum free DMEM. Secreted proteins were harvested after 48 hours by collecting conditional medium. Conditional medium was clarified by centrifugation at 10,000 g for 20 min and filtered through 0.2μ filter and used for purification.

C. Chromatographic Separation of EphB4 Ectodomain and EphB4 Ectodomain-HSA Fusion Protein

The EphB4 ectodomain fused to HSA was purified as follows: 700 ml of media was harvested from transiently transfected 293 cells grown in serum free media and concentrated up to final volume of 120 ml. Membrane: (Omega, 76 mm), 50 kDa C/O. After concentration, pH of the sample was adjusted by adding 6 ml of 1M NaAc, pH 5.5. Then sample was dialyzed against starting buffer (SB): 20 mM NaAc, 20 mM NaCl, pH 5.5 for O/N. 5 ml of SP-Sepharose was equilibrated with SB and sample was loaded. Washing: 100 ml of SB. Elution by NaCl: 12 ml/fraction and increment of 20 mM. Most of the EphrinB2 binding activity eluted in the 100 mM and 120 mM fractions.

Fractions, active in EphrinB2 binding assay (See SP chromatography, fractions # 100-120 mM) were used in second step of purification on Q-column. Pulled fractions were dialyzed against starting buffer #2 (SB2): 20 mM Tris-HCl, 20 mM NaCl, pH 8 for O/N and loaded onto 2 ml of Q-Sepharose. After washing with 20 ml of SB2, absorbed protein was eluted by NaCl: 3 ml/fraction with a concentration increment of 25 mM. Obtained fractions were analyzed by PAGE and in Ephrin-B2 binding assay. The 200 mM and 225 mM fractions were found to contain the most protein and the most B2 binding activity.

Soluble EphB4 ectodomain protein was purified as follows: 300 ml of conditional medium (see: Cell culture and transfections) were concentrated up to final volume of 100 ml, using ultrafiltration membrane with 30 kDa C/O. After concentration, pH of the sample was adjusted by adding 5 ml of 1 M Na-Acetate, pH 5.5. Then sample was dialyzed against starting buffer (StB): 20 mM Na-Acetate, 20 mM NaCl, pH 5.5 for O/N. 5 ml of SP-Sepharose was equilibrated with StB and sample was loaded. After washing the column with 20 ml of StB, absorbed proteins were eluted by linear gradient of concentration of NaCl (20-250 mM and total elution volume of 20 column's volumes). Purity of the proteins was analyzed by PAGE.

D. Biotinylation of sB4 and sB4-HSA Fusion Protein.

Both soluble EphB4 ectodomain protein (sB4) and EphB4 ectodomain fused to HSA (HSA-sB4) were biotin labeled through carbohydrate chains using sodium meta-periodate as an oxidant and EZ-Link Biotin Hydrazide (PIERCE, Cat. # 21339) according to manufacture's protocol. The in vitro stability of the biotinylated sB4 protein was tested by incubating 2.0×10⁻⁹ with 40 μL of mouse serum at 37° C. for 0, 0.5, 1, 2 and 3 days. Two μL of magnetic beads and B2-AP was added for an extra hour at room temperature. After washing twice with buffer, pnPP was added for 1 hour. Biotinylated sB4 protein was found to very stable over three days, with less than 10% of the B2 binding activity being lost over that time.

E. Ephrin-B2 Binding Properties of B4-HSA

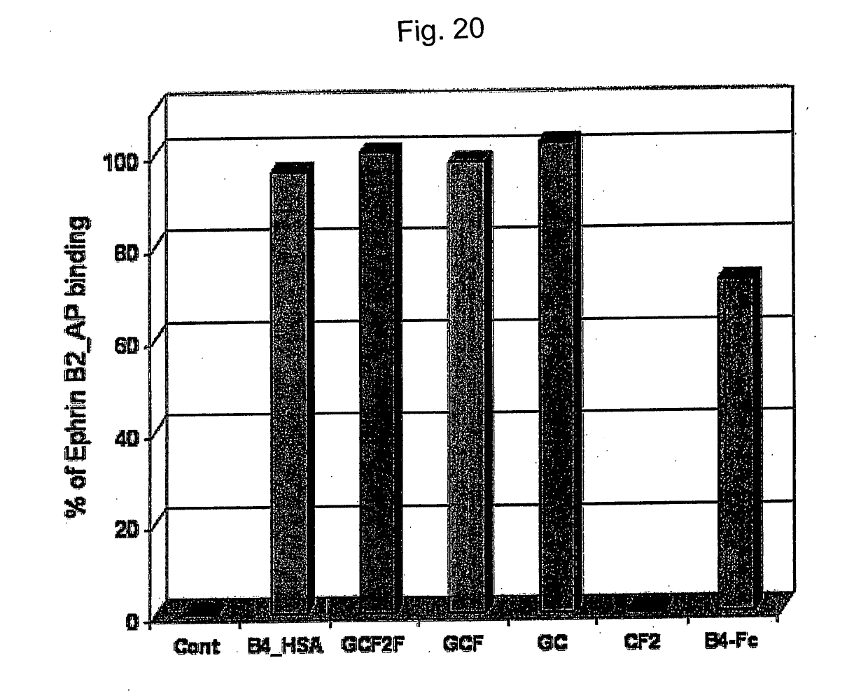

To test whether the B4-HSA fusion property retained the ability of the EphB4 extracellular domain to bind to EphrinB2, the ability of the purified B4-HSA fusion was compared to that of GCF2F, GCF2, GC, CF and B4-Fc fusion, which comprises the extracellular domain of B4 fused to hIgG1 Fc as described in Example 1. Biotinylated or His-tag protein samples were inoculated with the corresponding affinity magnetic beads and B2-AP for an hour at room temperature, before addition of PnPP. Results of binding assays are shown on FIG. 20. B4-HSA was found to retain most of its binding activity towards EphrinB2. Surprisingly, the B4-HSA protein was superior to the B4-Fc fusion in binding to EphrinB2.

An EphB4 ectodomain fusion to the C-terminus of HSA was also generated, and found to retain the ability to bind to EphrinB2 and was found to have enhanced stability in vivo over the EphB4 ectodomain.

F. Stability of B4-HSA vs. sB4 in Mice

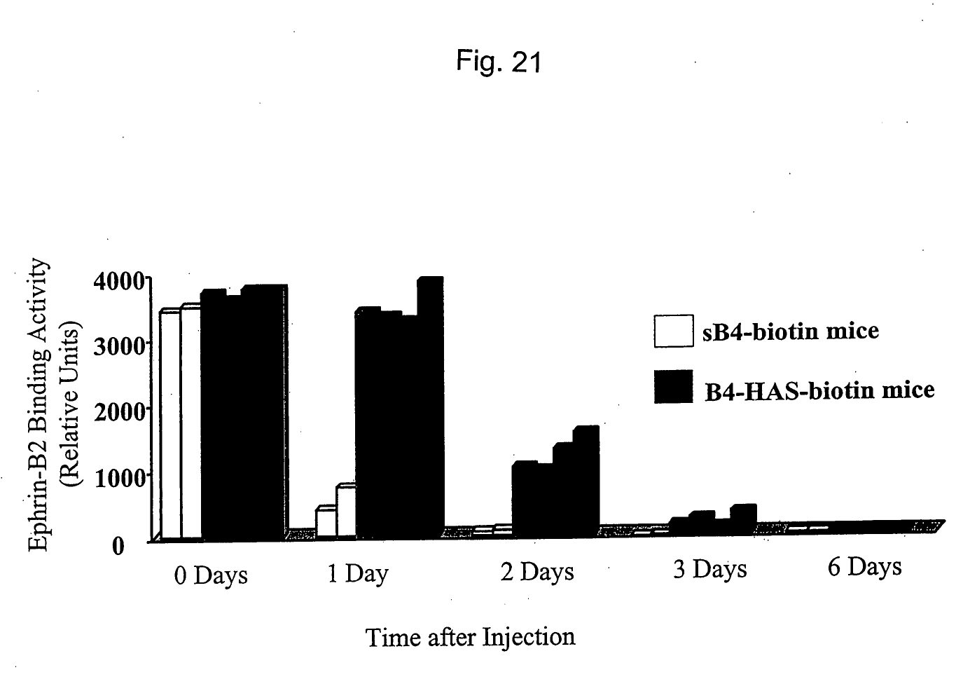

The stability of the purified biotinylated sB4 and sB4-HSA were assayed in vivo. Each of the proteins were intravenously injected into the tail of mice in the amount of 0.5 nmoles per mouse. Blood from the eye of each mouse was taken in time frames of 15 min (0 days), 1, 2, 3 and 6 days. 10 ml of obtained serum was used in binding assay with Ephrin-B2-Alkaline Phosphatase fusion protein and Streptavidin-coated magnetic beads as a solid phase. The stability of the two proteins is shown on FIG. 21. sB4-HSA was found to have superior stability relative to sB4. For example, one day after injection, the levels of sB4-HSA in the blood of the mice were 5-fold greater than those of sB4.

G. PEGylation of Biotinylated sB4

Prior to PEGylation, biotinylated sB4 protein generated as described above was concentrated up to final concentration of 2 mg/ml using a 30 kDa MWCO ultra membrane. Sample was dialyzed O/N against coupling buffer: 30 mM phosphate, 75 mM NaCl, pH 8.00. Coupling to PEG was performed at 4° C. for 18 hours in 10 fold molar excess of reactive linear PEG unless otherwise indicated. The reactive PEG used was PEG-succinimidyl propionate, having a molecular weight of about 20 kDa. Coupling to PEG may be similarly performed using branches PEGs, such as of 10 kDa, 20 kDa or 40 kDa. Other linear PEG molecules of 10 or 40 kDa may also be used.

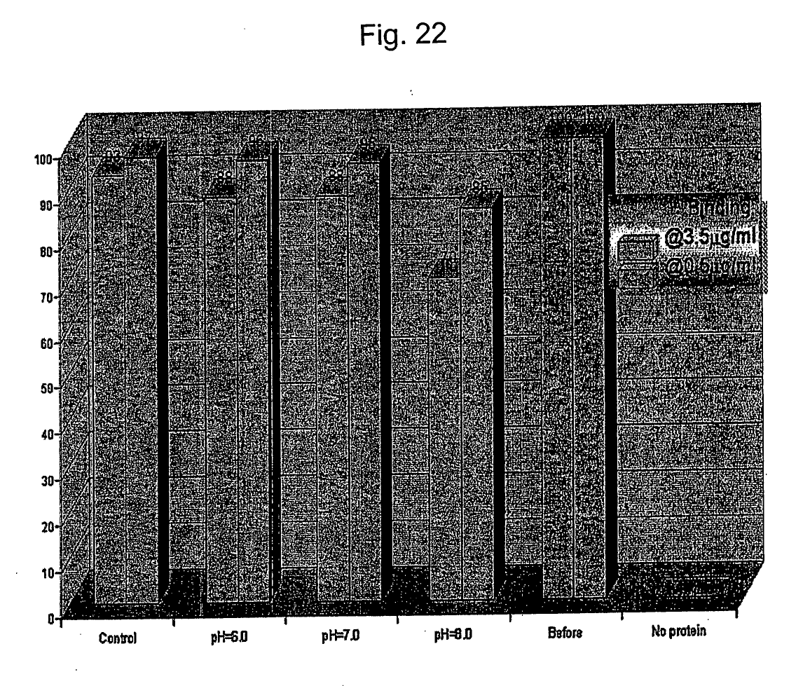

After PEGylation, the protein sample containing EphB4 ectodomain was dialyzed against StB O/N. Three ml of SP-Sepharose was equilibrated with StB and sample was loaded. Washing and elution of absorbed proteins was performed as above (see: Purification of soluble EphB4 ectodomain and its fusion to HSA) with just one modification: total elution volume was 40 volumes of column. FIG. 22 shows chromatographic separation of PEG derivatives of EphB4 protein on SP-Sepharose columns. Purity of the PEG-modified EphB4 protein was analyzed by SDS-PAGE.

Double modified (PEGylated Biotinylated)ₛB4 was used on ion-exchange chromatography to separate non-PEGylated, mono-PEGylated and poly-PEGylated proteins from each other. Pegylated sample was dialyzed O/N against 20 mM Na-acetate, 20 mM NaCl, pH 5.5 and loaded onto 2 ml of SP-Sepharose. After washing with 10 ml of buffer, absorbed proteins were separated by gradual elution of NaCl: 3 ml/fraction and increment of 25 mM NaCl. Obtained fractions were analyzed by PAGE and in Ephrin-B2 binding assay.

H. Effect of PEGylation Conditions on sB4 Binding to EphrinB2

The effects of pegylating biotinylated sB4 under different pH conditions was determined. sB4 was pegylated at pH 6, 7 or 8, and the pegylated products were tested for binding to EphrinB2 as shown in FIG. 22. Ephrin2B binding activity was retained when PEGylation was performed at pH 6 and pH 7, but was partially lost at pH 8.

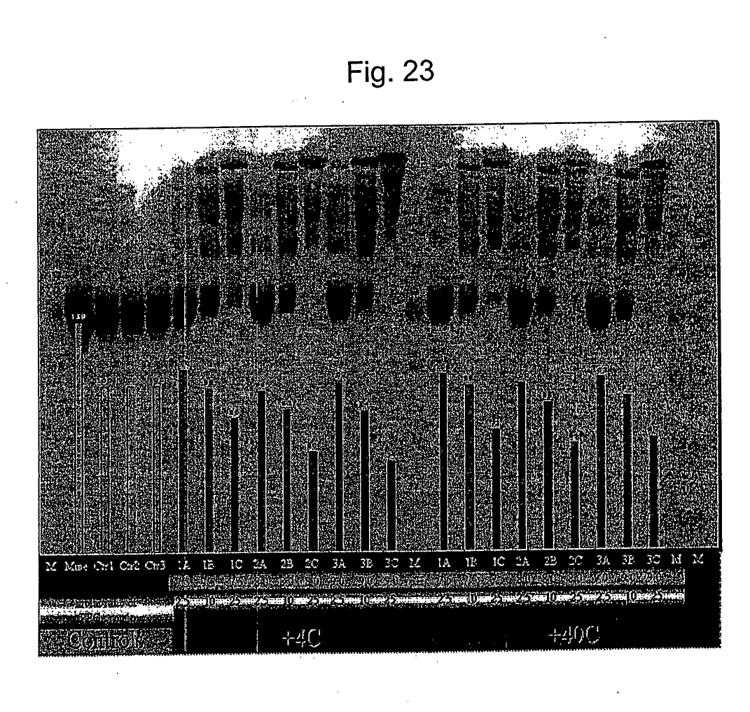

Additional combinations of parameters were tested, including temperature, pH and molar ratio of pegylation agent to sB4 protein, and the ability of the products of the pegylation reaction to bind to Ephrin-B2. The results of the optimization experiment are shown in FIG. 23. These results confirm the gradual decrease in B2 binding activity at basic pH.

I. Purification of Pegylated sB4 Species

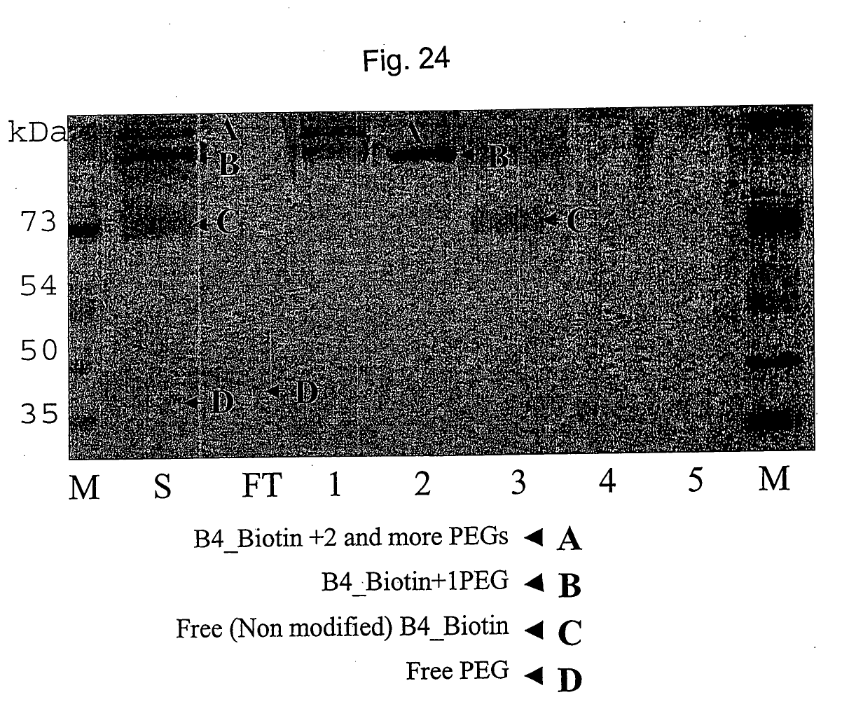

Biotinylated sB4 protein was concentrated up to final concentration of 2 mg/ml using a 30 kDa MWCO ultra membrane. Sample was dialyzed O/N against coupling buffer: 30 mM phosphate, 75 mM NaCl, pH 8.00. Coupling to PEG was performed at 4° C. for 18 hours in 10 fold molar excess of reactive PEG. Double modified (PEGylated Biotinylated) sB4 was used on ion-exchange chromatography to separate non-PEGylated, mono-PEGylated and poly-PEGylated proteins from each other. Sample was dialyzed for O/N against 20 mM Na-Acetate, 20 mM NaCl, pH 5.5 and loaded onto 2 ml of SP-Sepharose. After washing with 10 ml of buffer, absorbed proteins were separated by gradual elution of NaCl: 3 ml/fraction and increment of 25 mM NaCl. Obtained fractions were analyzed by PAGE as shown in FIG. 24. Fractions 1, 2 and 3 were found to correspond to polypegylated, monopegylated and unpegylated biotinylated sB4.

J. In Vitro Properties of PEGylated EphB4 Derivatives

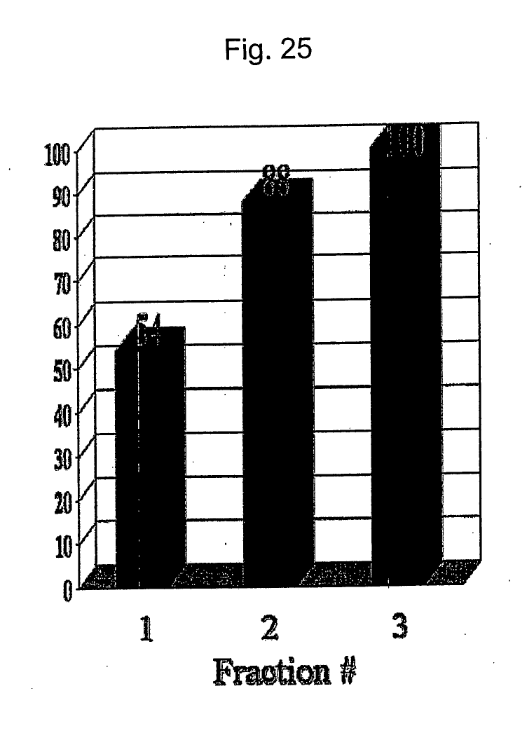

Fractions 1, 2 and 3 of biotinylated and PEGylated sB4 from the SP column purification, corresponding to polypegylated, monopegylated and unpegylated biotinylated sB4, were tested for their ability to bind EphrinB2 using the standard assay. Results of this experiment are shown on FIG. 25. The order of binding activity was found to be Unpegylated>monopegylated>polypegylated.

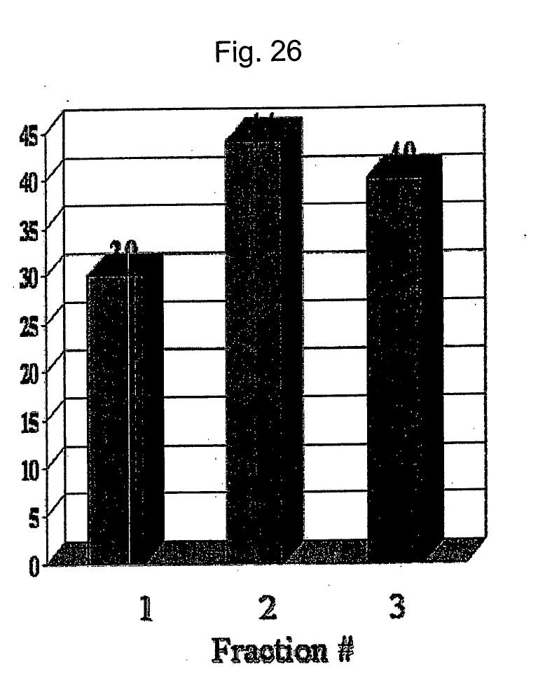

The fractions were also tested for their stability in vitro. The fractions were tested for retention of EphrinB2 binding activity after incubation in mouse serum at 37° C. for three days. The results of this experiment are shown in FIG. 26. The order of in vitro stability was found to be monopegylated>unpegylated>polypegylated.

K. In Vivo Stability Analysis of PEGylated Derivatives of EphB4 Ectodomain in Mice

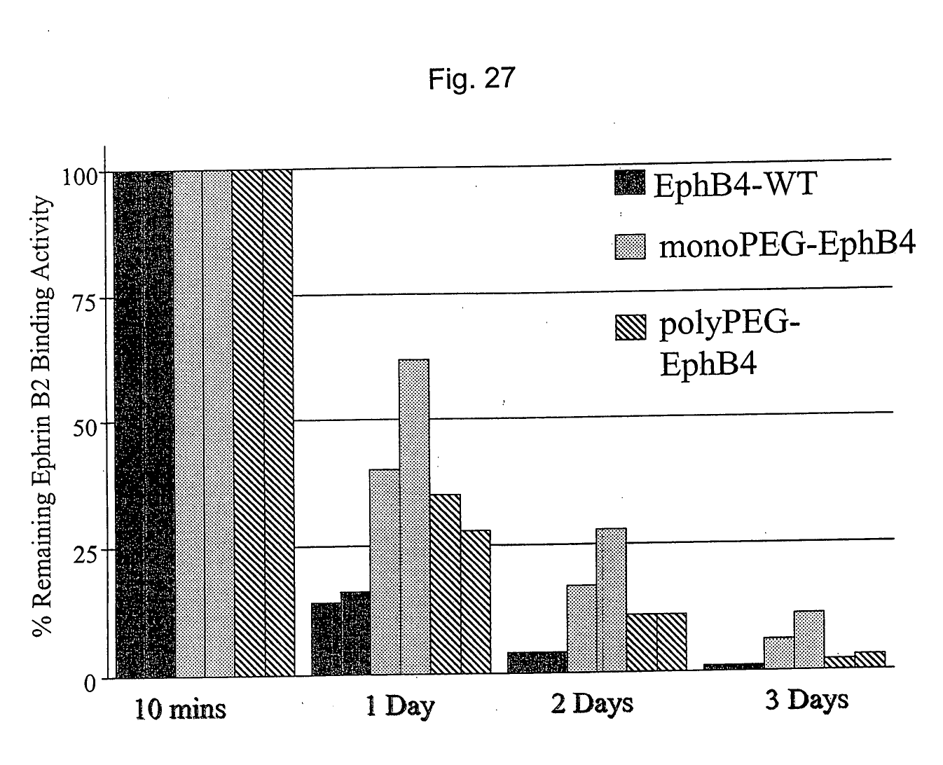

Fractions 1, 2 and 3 of biotinylated and PEGylated sB4 from the SP column purification, corresponding to polypegylated, monopegylated and unpegylated biotinylated sB4, were introduced by intravenous injection into mice in the amount of 0.5 nMoles/mouse. Blood from each mouse was taken in time frame of 10 min, 1, 2 and 3 days. 10 ml of obtained serum was used in binding assay with Ephrin-B2-Alkaline Phosphatase fusion protein and Streptavidin-coated magnetic beads as a solid phase. Signals, obtained at 10 min were taken as 100%. The two mice for each protein were of a different strain. Results are shown in FIG. 27. Pegylation was found to increase the stability of EphB4 in vivo relative to unpegylated EphB4.

### Example 3

### Inhibition the Fusion of NIPA and Hendra virus to the Endothelia Cells

Cell fusion assays were used for identification of agents that block viral entry in the target cells. The results correlate with inhibition of viral infectivity of target cells. Target cells express EphrinB2 or related receptors which can function as the receptor for viruses such as Nipah and Hendra.

Fusion between HeV and NiV F and G envelope glycoprotein-expressing cells (effector cells) and target cells was measured by two assays. The first one was a reporter gene assay, in which the cytoplasm of one cell population contained vaccinia virus-encoded T7 RNA polymerase and the cytoplasm of the other contained the Escherichia coli lacZ gene linked to the T7 promoter (β-galactosidase) was synthesized only in fused cells (Bossart and Broder. 2004. Methods Mol. Biol 269:309-332; Nussbaum, et al. 1994. J. Virol. 68:5411-5422). The second one was a syncytium assay. Typically, the expression of HeV and NiV F and G was performed in a HeV and NiV fusion and an infection-negative HeLa cell line derivative (HeLa-USU). Cytogenetic analysis confirmed that the HeLa-USU cell line resistant to NiV and HeV mediated membrane fusion, and live virus infection was derived from the ATCC(CCL-2) HeLa cell line (data not shown). Vaccinia virus-encoded proteins (Bossart, et al. 2001. Virology 290:121-135) were produced by infecting cells at an MOI of 10 and incubating infected cells at 31° C. overnight. Cell fusion reactions were conducted with the various cell mixtures in 96-well plates at 37° C. Typically, the ratio of envelope glycoprotein-expressing cells to target cells was 1:1 (2×10⁵ total cells per well; 0.2-ml total volume). Cytosine arabinoside (40 μg/ml) was added to the fusion reaction mixture to reduce nonspecific β-Gal production. For quantitative analyses, Nonidet P-40 was added (0.5% final) at 2.5 h, and aliquots of the lysates were assayed for β-Gal at ambient temperature with the substrate chlorophenol red-D-galactopyranoside (Roche Diagnostics Corp., Indianapolis, Ind.). For inhibition by antibodies, serial antibody dilutions were made and added to effector cell populations 30 min prior to the addition of target cell populations. All assays were performed in duplicate, and fusion data were calculated and expressed as rates of β-Gal activity (change in OD at 570 nm per minute×1,000) (Nussbaum, et al. 1994. J. Virol. 68:5411-5422). They were normalized with respect to cell fusion in the absence of antibodies and plotted as function of the antibody concentration. Representative results were summarized in Tables 1 and 2 below.

TABLE 1
Percentage fusion in presence of 10 μg/ml of each unknown protein.

| Protein No. | Protein Name | % fusion | St. dev. |
|---|---|---|---|
| | Positive Control | 100.00 | 2.05 |
| No. 1 | Inactivated | 83.33 | 1.10 |
| | Human EphrinB2-Ec | | |
| No. 2 | human EphrinB2EC-Human | 54.76 | 1.07 |
| | serum albumin fusion protein | | |
| No. 3 | Human EphrinB2-Fc | 15.24 | 0.43 |
| No. 4 | Murine EphrinA2-EC-Fc | 116.67 | 1.17 |
| No. 5 | Murine EphrinB1-EC-Fc | 140.48 | 1.17 |
| No. 6 | Bovine Serum Albumin | 126.19 | 1.57 |
| No. 7 | Murine EphrinB2-Ec-Fc | 8.33 | 0.33 |

TABLE 2
Percentage fusion in presence of each unknown protein,
EphrinB2/Fc chimera, IgG M102.4, and NiV FC2 peptide
as controls for complete inhibition.

| | 10 μg/ml | | 1.0 μg/ml | | 0.1 μg/ml | |
|---|---|---|---|---|---|---|
| | % | | % | | % | |
| Inhibitor | fusion | St. dev. | fusion | St. dev. | fusion | St. dev. |
| No. 1 | 77.33 | 8.93 | 104.00 | 2.53 | 102.67 | 11.20 |
| No. 2 | 64.00 | 3.47 | 114.67 | 4.00 | 85.33 | 4.67 |
| No. 3 | 30.67 | 4.27 | 96.00 | 11.20 | 84.00 | 5.73 |
| No. 4 | 112.00 | 10.00 | 104.00 | 2.40 | 81.33 | 5.07 |
| No. 5 | 146.67 | 12.27 | 133.33 | 8.93 | 109.33 | 2.53 |
| No. 6 | 124.00 | 16.00 | 112.00 | 3.33 | 112.00 | 2.13 |
| No. 7 | 2.93 | 1.13 | 1.87 | 1.27 | 70.67 | 4.53 |

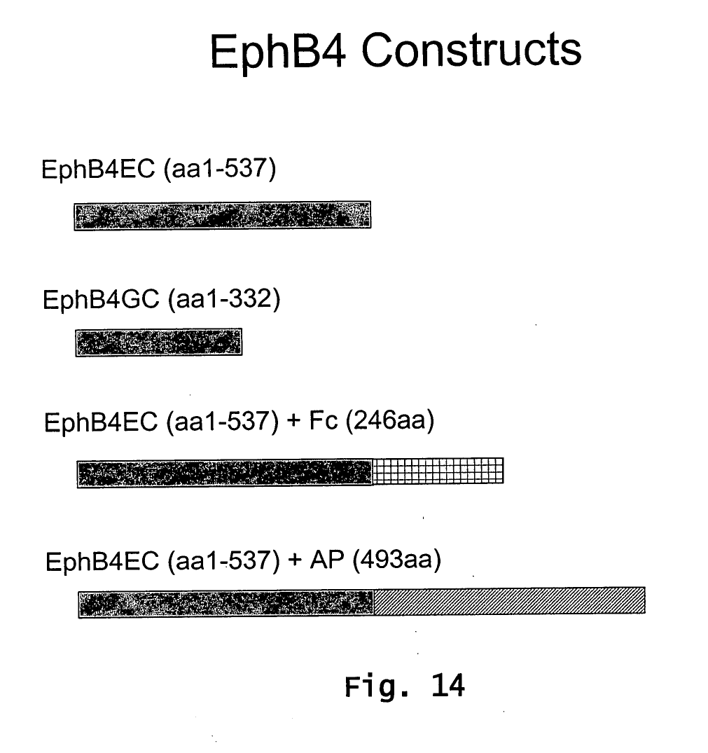

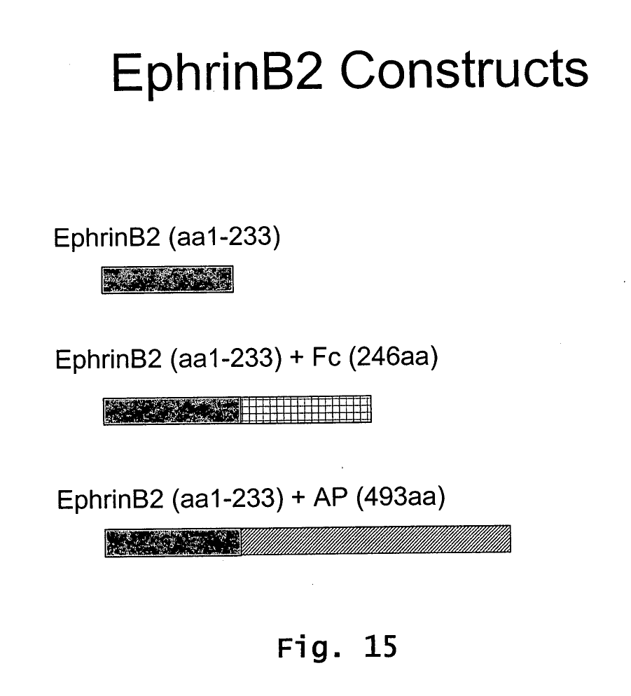

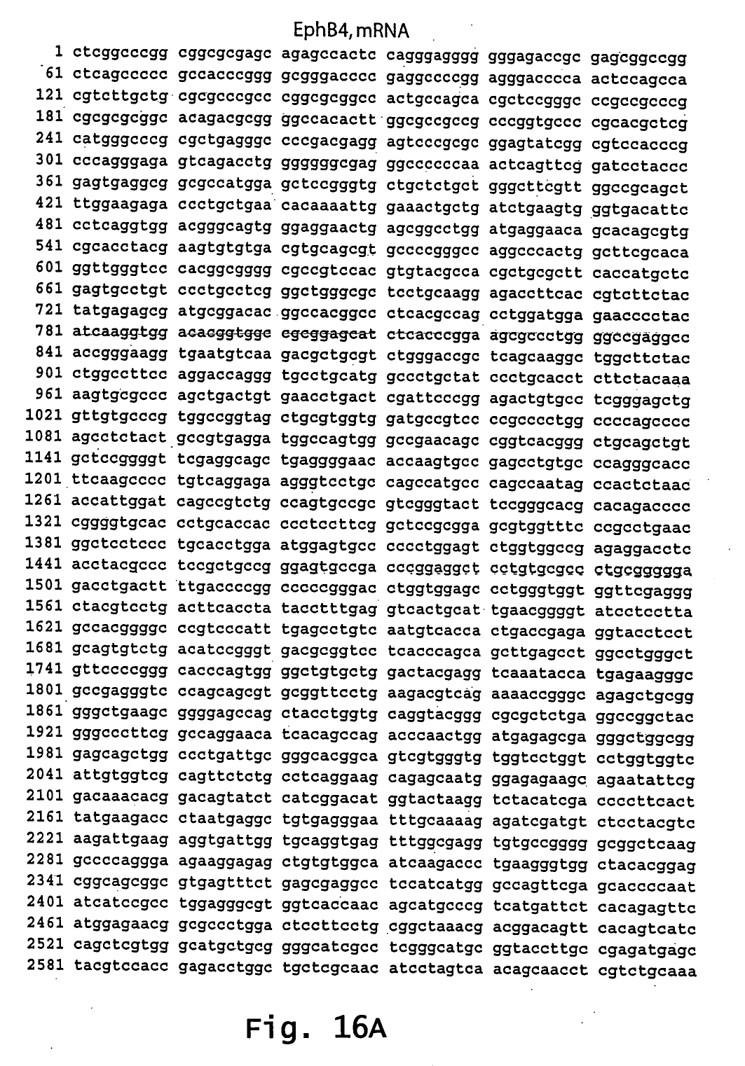

FIGS. 14 and 15 show further examples of EphB4 and EphrinB2 protein constructs that were tested in these assays, including EphB4EC (SEQ ID NO: 16), EphB4-GC (SEQ ID NO: 12), EphB4EC-Fc fusion from pIG-Fc vector (SEQ ID NO: 4), EphrinB2EC (SEQ ID NO: 3), EphrinB2EC-Fc fusion from pCXFc vector (SEQ ID NO: 5), and two AP fusions below:

EphB4EC-AP fusion from pAPTag2 vector (SEQ ID NO: 6):

MELRVLLCWASLAAALEETLLNTKLETADLKWVTFPQVDGQWEELSGLDE
EQHSVRTYEVCEVQRAPGQAHWLRTGWVPRRGAVHVYATLRFTMLECLSL
PPAGRSCKETFTVFYYESDADTATALTPAWMENPYIKVDTVAAEHLTRKR
PGAEATGKVNVKTLRLGPLSKAGFYLAFQDQGACMALLSLHLFYKKCAQL
TVNLTRFPETVPRELVVPVAGSCVVDAVPAPGPSPSLYCREDGQWAEQPV
TGCSCAPGFEAAEGNTKCRACAQGTFKPLSGEGSCQPCPANSHSNTIGSA
VCQCRVGYFRARTDPRGAPCTTPPSAPRSVVSRLNGSSLHLEWSAPLESG
GREDLTYALRCRECRPGGSCAPCGGDLTFDPGPRDLVEPWVVVRGLRPDF
TYTFEVTALNGVSSLATGPVPFEPVNVTTDREVPPAVSDIRVTRSSPSSL
SLAWAVPRAPSGAWLDYEVKYHEKGAEGPSSVRFLKTSENRAELRGLKRG
ASYLVQVRARSEAGYGPFGQEHHSQTQLDESEGWREQGSSGIIPVEEENP
DFWNREAAEALGAAKKLQPAQTAAKNLIIFLGDGMGVSTVTAARILKGQK
KDKLGPEIPLAMDRFPYVALSKTYNVDKHVPDSGATATAYLCGVKGNFQT
IGLSAAARFNQCNTTRGNEVISVMNRAKKAGKSVGVVTTTRVQHASPAGT
YAHTVNRNWYSDADVPASARQEGCQDIATQLISNMDIDVILGGGRKYMFP
MGTPDPEYPDDYSQGGTRLDGKNLVQEWLAKRQGARYVWNRTELMQASLD
PSVTHLMGLFEPGDMKYEIHRDSTLDPSLMEMTEAALRLLSRNPRGFFLF
VEGGRIDHGHHESRAYPALTETIMFDDAIERAGQLTSEEDTLSLVTADHS
HVFSFGGYPLRGSSIFGLAPGKARDRKAYTVLLYGNGPGYVLKDGARPDV
TESESGSPEYRQQSAVPLDEETHAGEDVAVFARGPQAHLVHGVQEQTFIA
HVMAFAACLEPYTACDLAPPAGTTDAAHPG

EphrinB2EC-AP from pAPTag2 (SEQ ID NO: 7):

MAVRRDSVWKYCWGVLMVLCRTAISKSIVLEPIYWNSSNSKFLPGQGLVL
YPQIGDKLDIICPKVDSKTVGQYEYYKVYMVDKDQADRCTIKKENTPLLN
CAKPDQDIKFTIKFQEFSPNLWGLEFQKNKDYYIISTSNGSLEGLDNQEG
GVCQTRAMKILMKVGQDASSAGSTRNKDPTRRPELEAGTNGRSSTTSPFV
KPNPGSSTDGNSAGHSGNNILGSEGSSGIIPVEEENPDFWNREAAEALGA
AKKLQPAQTAAKNLIIFLGDGMGVSTVTAARILKGQKKDKLGPEIPLAMD
RFPYVALSKTYNVDKHVPDSGATATAYLCGVKGNFQTIGLSAAARFNQCN
TTRGNEVISVMNRAKKAGKSVGVVTTTRVQHASPAGTYAHTVNRNWYSDA
DVPASARQEGCQDIATQLISNMDIDVILGGGRKYMFPMGTPDPEYPDDYS
QGGTRLDGKNLVQEWLAKRQGARYVWNRTELMQASLDPSVTHLMGLFEPG
DMKYEIHRDSTLDPSLMEMTEAALRLLSRNPRGFFLFVEGGRIDHGHHES
RAYRALTETIMFDDAIERAGQLTSEEDTLSLVTADHSHVFSFGGYPLRGS
SIFGLAPGKARDRKAYTVLLYGNGPGYVLKDGARPDVTESESGSPEYRQQ
SAVPLDEETHAGEDVAVFARGPQAHLVHGVQEQTFIAHVMAFAACLEPYT
ACDLAPPAGTTDAAHPG

In sum, the experiments demonstrated the neutralizing activity of EphrinB2 proteins to viral entry to target cells using a cell fusion model. Specifically, EphrinB2 proteins inhibited the fusion of NIPA and Hendra virus to the endothelial cells. Among the EphrinB2 proteins, dimeric forms of EphrinB3 proteins (e.g, Fc fusions) exhibited higher neutralizing activity against the viral entry than the monomeric forms of EphrinB2 proteins. Further, EphB4 proteins were also active in inhibiting viral entry to endothelial cells, although they were less active than EphrinB2 proteins (data not shown).

Incorporation By Reference

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for treating or preventing infection by an EphrinB2-binding virus of the family Paramyxoviridae, the method comprising administering to a subject in need thereof an effect amount of an agent selected from the group consisting of:
(a) a soluble polypeptide comprising an amino acid sequence of an extracellular domain of an EphB4 protein, wherein the EphB4 polypeptide is a monomer and binds specifically to an EphrinB2 polypeptide;
(b) a soluble polypeptide comprising an amino acid sequence of an extracellular domain of an EphrinB2 protein, wherein the soluble EphrinB2 polypeptide is a monomer and binds with high affinity to an EphB4 polypeptide;
(c) an antibody which binds to an extracellular domain of an EphB4 protein and inhibits an activity of the EphB4 protein;
(d) an antibody which binds to an extracellular domain of an EphrinB2 protein and inhibits an activity of the EphrinB2;
(e) a nucleic acid compound comprising at least a portion that hybridizes to an EphB4 transcript under physiological conditions and decreases the expression of EphB4 in a cell; and
(f) a nucleic acid compound comprising at least a portion that hybridizes to an EphrinB2 transcript under physiological conditions and decreases the expression of EphrinB2 in a cell.

2. The method of claim 1, wherein the soluble polypeptide of (a) or (b) comprises a modification that increases serum half-life.

3. The method of claim 2, wherein said modification comprises a polyethylene glycol group.

4. The method of claim 1, wherein the soluble polypeptide of (a) or (b) is a fusion protein.

5. The method of claim 4, wherein the polypeptide comprises an albumin protein or fragments thereof.

6. The method of claim 5, wherein said albumin protein is selected from the group consisting of a human serum albumin (HSA) and bovine serum albumin (BSA).

7. The method of claim 1, wherein the virus is a Henipavirus.

8. The method of claim 7, wherein the Henipavirus is Nipah or Hendra virus.

9. A method for inhibiting fusion between a virus of the family Paramyxoviridae and a target cell, comprising contacting the cell with an effect amount of an agent selected from the group consisting of:
(a) a soluble polypeptide comprising an amino acid sequence of an extracellular domain of an EphB4 protein, wherein the EphB4 polypeptide is a monomer and binds specifically to an EphrinB2 polypeptide;
(b) a soluble polypeptide comprising an amino acid sequence of an extracellular domain of an EphrinB2 protein, wherein the soluble EphrinB2 polypeptide is a monomer and binds with high affinity to an EphB4 polypeptide;
(c) an antibody which binds to an extracellular domain of an EphB4 protein and inhibits an activity of the EphB4 protein;
(d) an antibody which binds to an extracellular domain of an EphrinB2 protein and inhibits an activity of the EphrinB2;
(e) a nucleic acid compound comprising at least a portion that hybridizes to an EphB4 transcript under physiological conditions and decreases the expression of EphB4 in a cell; and
(f) a nucleic acid compound comprising at least a portion that hybridizes to an EphrinB2 transcript under physiological conditions and decreases the expression of EphrinB2 in a cell.

10. The method of claim 9, wherein the virus is a Henipavirus.

11. The method of claim 10, wherein the Henipavirus is Nipah or Hendra virus.

12. The method of claim 9, wherein the target cell is an endothelial cell.


SEQUENCE LISTING (115 sequences)

SEQ ID NO: 1 - protein, 570 aa, Unknown
  Recombinant B4ECv3 protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGSKRAILQIEGKPIPNPLLGLDSTRTGHHHHHH

SEQ ID NO: 2 - protein, 555 aa, Unknown
  Recombinant B4ECv3NT protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGSKRAILQISSTVAAARV

SEQ ID NO: 3 - protein, 233 aa, Unknown
  Recombinant B2EC protein
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVGSHHHHHH

SEQ ID NO: 4 - protein, 771 aa, Unknown
  Recombinant B4ECv3-FC protein
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQDPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 459 aa, Unknown
  Recombinant B2EC-FC protein
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVDPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 1030 aa, Unknown [sequence omitted]

SEQ ID NO: 7 - protein, 717 aa, Unknown [sequence omitted]

SEQ ID NO: 8 - DNA, 4235 bp, Homo sapiens
ctcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggt

SEQ ID NO: 9 - DNA, 4335 bp, Homo sapiens
gcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaaaaaaaaaaaaaaa

SEQ ID NO: 10 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 11 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 12 - protein, 332 aa, Unknown
  Recombinant B4EC-GC
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAHHHHHH

SEQ ID NO: 13 - protein, 431 aa, Unknown
  Recombinant GCF
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVHHHHHH

SEQ ID NO: 14 - protein, 570 aa, Unknown; identical to SEQ ID NO: 1
  Recombinant FL-hB4EC

SEQ ID NO: 15 - protein, 401 aa, Unknown
  Recombinant B4-CF2
MELRVLLCWASLAAALEETLLNTKLETQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQGGRSSLEGPRFEGKPIPNPLLGLDSTRTGHHHHHH

SEQ ID NO: 16 - protein, 537 aa, Unknown
  Recombinant B4-GCF2F
MELRVLLCWASLAAALEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQ

SEQ ID NO: 17 - protein, 522 aa, Unknown
  Recombinant processed B4-GCF2F
LEETLLNTKLETADLKWVTFPQVDGQWEELSGLDEEQHSVRTYEVCEVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAWLDYEVKYHEKGAEGPSSVRFLKTSENRAELRGLKRGASYLVQVRARSEAGYGPFGQEHHSQTQLDESEGWREQ

SEQ ID NO: 18 - protein, 1124 aa, Unknown
  Recombinant HSA-EphB4 precursor protein
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQSRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 1109 aa, Unknown
  Recombinant HSA-EphB4 mature protein
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQSRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 20 - DNA, 9244 bp, Artificial Sequence
  PEF6-GCF2 plasmid sequence
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattgcccttcaagcttgccgccaccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgcctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgtcgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagtctagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttaagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataatagcggccgcttaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgtcgacaggtgcttctcgatctgcatcctgggatcaaagcgatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 21 - DNA, 20 bp, Unknown
  Oligonucleotide
tcagaccttgtagtaaatgt

SEQ ID NO: 22 - DNA, 20 bp, Unknown
  Oligonucleotide
tcgccgggctctgcgggggc

SEQ ID NO: 23 - DNA, 20 bp, Unknown
  Oligonucleotide
atctcctggacgatgtacac

SEQ ID NO: 24 - DNA, 20 bp, Unknown
  Oligonucleotide
cgggtgcccgtagtccccgc

SEQ ID NO: 25 - DNA, 20 bp, Unknown
  Oligonucleotide
tgaccttctcgtagtgaggg

SEQ ID NO: 26 - DNA, 20 bp, Unknown
  Oligonucleotide
cagaagacgctgtccgcagt

SEQ ID NO: 27 - DNA, 20 bp, Unknown
  Oligonucleotide
ccttagcgggatgataatgt

SEQ ID NO: 28 - DNA, 20 bp, Unknown
  Oligonucleotide
cactgggctctgagccgttg

SEQ ID NO: 29 - DNA, 20 bp, Unknown
  Oligonucleotide
ttgttgccgctgcgcttggg

SEQ ID NO: 30 - DNA, 20 bp, Unknown
  Oligonucleotide
tgtggccagtgtgctgagcg

SEQ ID NO: 31 - DNA, 20 bp, Unknown
  Oligonucleotide
acagcgtggtcgtgtgctgc

SEQ ID NO: 32 - DNA, 20 bp, Unknown
  Oligonucleotide
ggcgagtgcttcctgtgtct

SEQ ID NO: 33 - DNA, 20 bp, Unknown
  Oligonucleotide
cctccggtacttcagcaaga

SEQ ID NO: 34 - DNA, 20 bp, Unknown
  Oligonucleotide
ggaccaccagcgtgatgatg

SEQ ID NO: 35 - DNA, 20 bp, Unknown
  Oligonucleotide
atgacgatgaagatgatgca

SEQ ID NO: 36 - DNA, 20 bp, Unknown
  Oligonucleotide
tcctgaagcaatccctgcaa

SEQ ID NO: 37 - DNA, 20 bp, Unknown
  Oligonucleotide
ataaggccacttcggaaccg

SEQ ID NO: 38 - DNA, 20 bp, Unknown
  Oligonucleotide
aggatgttgttccccgaatg

SEQ ID NO: 39 - DNA, 20 bp, Unknown
  Oligonucleotide
tccggcgctgttgccgtctg

SEQ ID NO: 40 - DNA, 20 bp, Unknown
  Oligonucleotide
tgctagaacctggatttggt

SEQ ID NO: 41 - DNA, 20 bp, Unknown
  Oligonucleotide
tttacaaagggacttgttgt

SEQ ID NO: 42 - DNA, 20 bp, Unknown
  Oligonucleotide
cgaacttcttccatttgtac

SEQ ID NO: 43 - DNA, 20 bp, Unknown
  Oligonucleotide
cagcttctagttctggacgt

SEQ ID NO: 44 - DNA, 20 bp, Unknown
  Oligonucleotide
cttgttggatctttattcct

SEQ ID NO: 45 - DNA, 20 bp, Unknown
  Oligonucleotide
ggttgatccagcagaacttg

SEQ ID NO: 46 - DNA, 20 bp, Unknown
  Oligonucleotide
catcttgtccaactttcatg

SEQ ID NO: 47 - DNA, 20 bp, Unknown
  Oligonucleotide
aggatcttcatggctcttgt

SEQ ID NO: 48 - DNA, 20 bp, Unknown
  Oligonucleotide
ctggcacacccctccctcct

SEQ ID NO: 49 - DNA, 20 bp, Unknown
  Oligonucleotide
ggttatccaggccctccaaa

SEQ ID NO: 50 - DNA, 20 bp, Unknown
  Oligonucleotide
gacccatttgatgtagatat

SEQ ID NO: 51 - DNA, 20 bp, Unknown
  Oligonucleotide
aatgtaataatctttgttct

SEQ ID NO: 52 - DNA, 20 bp, Unknown
  Oligonucleotide
tctgaaattctagaccccag

SEQ ID NO: 53 - DNA, 20 bp, Unknown
  Oligonucleotide
aggttagggctgaattcttg

SEQ ID NO: 54 - DNA, 20 bp, Unknown
  Oligonucleotide
aaacttgatggtgaatttga

SEQ ID NO: 55 - DNA, 20 bp, Unknown
  Oligonucleotide
tatcttggtctggtttggca

SEQ ID NO: 56 - DNA, 20 bp, Unknown
  Oligonucleotide
cagttgaggagaggggtatt

SEQ ID NO: 57 - DNA, 20 bp, Unknown
  Oligonucleotide
ttccttcttaatagtgcatc

SEQ ID NO: 58 - DNA, 20 bp, Unknown
  Oligonucleotide
tgtctgcttggtctttatca

SEQ ID NO: 59 - DNA, 20 bp, Unknown
  Oligonucleotide
accatataaactttataata

SEQ ID NO: 60 - DNA, 20 bp, Unknown
  Oligonucleotide
ttcatactggccaacagttt

SEQ ID NO: 61 - DNA, 20 bp, Unknown
  Oligonucleotide
tagagtccactttggggcaa

SEQ ID NO: 62 - DNA, 20 bp, Unknown
  Oligonucleotide
ataatatccaatttgtctcc

SEQ ID NO: 63 - DNA, 20 bp, Unknown
  Oligonucleotide
tatctgtgggtatagtacca

SEQ ID NO: 64 - DNA, 20 bp, Unknown
  Oligonucleotide
gtccttgtccaggtagaaat

SEQ ID NO: 65 - DNA, 20 bp, Unknown
  Oligonucleotide
ttggagttcgaggaattcca

SEQ ID NO: 66 - DNA, 20 bp, Unknown
  Oligonucleotide
atagataggctctaaaacta

SEQ ID NO: 67 - DNA, 20 bp, Unknown
  Oligonucleotide
tcgatttggaaatcgcagtt

SEQ ID NO: 68 - DNA, 20 bp, Unknown
  Oligonucleotide
ctgcataaaaccatcaaaac

SEQ ID NO: 69 - DNA, 20 bp, Unknown
  Oligonucleotide
accccagcagtacttccaca

SEQ ID NO: 70 - DNA, 20 bp, Unknown
  Oligonucleotide
cggagtcccttctcacagcc

SEQ ID NO: 71 - DNA, 20 bp, Unknown
  Oligonucleotide
gagtcccttctcacagccat

SEQ ID NO: 72 - DNA, 21 bp, Unknown
  Oligonucleotide
aactgcgatttccaaatcgat

SEQ ID NO: 73 - DNA, 21 bp, Unknown
  Oligonucleotide
aactccaaatttctacctgga

SEQ ID NO: 74 - DNA, 21 bp, Unknown
  Oligonucleotide
aatttctacctggacaaggac

SEQ ID NO: 75 - DNA, 21 bp, Unknown
  Oligonucleotide
aaatttctacctggacaagga

SEQ ID NO: 76 - DNA, 21 bp, Unknown
  Oligonucleotide
aaggactggtactatacccac

SEQ ID NO: 77 - DNA, 21 bp, Unknown
  Oligonucleotide
aagtggactctaaaactgttg

SEQ ID NO: 78 - DNA, 21 bp, Unknown
  Oligonucleotide
aaactgttggccagtatgaat

SEQ ID NO: 79 - DNA, 21 bp, Unknown
  Oligonucleotide
aaaactgttggccagtatgaa

SEQ ID NO: 80 - DNA, 21 bp, Unknown
  Oligonucleotide
aagaccaagcagacagatgca

SEQ ID NO: 81 - DNA, 21 bp, Unknown
  Oligonucleotide
aaagaccaagcagacagatgc

SEQ ID NO: 82 - DNA, 21 bp, Unknown
  Oligonucleotide
aagtttcaagaattcagccct

SEQ ID NO: 83 - DNA, 21 bp, Unknown
  Oligonucleotide
aagaattcagccctaacctct

SEQ ID NO: 84 - DNA, 21 bp, Unknown
  Oligonucleotide
aattcagccctaacctctggg

SEQ ID NO: 85 - DNA, 21 bp, Unknown
  Oligonucleotide
aactgtgccaaaccagaccaa

SEQ ID NO: 86 - DNA, 21 bp, Unknown
  Oligonucleotide
aaatgggtctttggagggcct

SEQ ID NO: 87 - DNA, 21 bp, Unknown
  Oligonucleotide
aagatcctcatgaaagttgga

SEQ ID NO: 88 - DNA, 21 bp, Unknown
  Oligonucleotide
aaagttggacaagatgcaagt

SEQ ID NO: 89 - DNA, 21 bp, Unknown
  Oligonucleotide
aagagccatgaagatcctcat

SEQ ID NO: 90 - DNA, 21 bp, Unknown
  Oligonucleotide
aagttggacaagatgcaagtt

SEQ ID NO: 91 - DNA, 21 bp, Unknown
  Oligonucleotide
aagatgcaagttctgctggat

SEQ ID NO: 92 - DNA, 21 bp, Unknown
  Oligonucleotide
aagttctgctggatcaaccag

SEQ ID NO: 93 - DNA, 21 bp, Unknown
  Oligonucleotide
aaccaggaataaagatccaac

SEQ ID NO: 94 - DNA, 21 bp, Unknown
  Oligonucleotide
aaagatccaacaagacgtcca

SEQ ID NO: 95 - DNA, 21 bp, Unknown
  Oligonucleotide
aagatccaacaagacgtccag

SEQ ID NO: 96 - DNA, 21 bp, Unknown
  Oligonucleotide
aacaagacgtccagaactaga

SEQ ID NO: 97 - DNA, 21 bp, Unknown
  Oligonucleotide
aagacgtccagaactagaagc

SEQ ID NO: 98 - DNA, 21 bp, Unknown
  Oligonucleotide
aaatggaagaagttcgacaac

SEQ ID NO: 99 - DNA, 21 bp, Unknown
  Oligonucleotide
aactagaagctggtacaaatg

SEQ ID NO: 100 - DNA, 21 bp, Unknown
  Oligonucleotide
aatggaagaagttcgacaaca

SEQ ID NO: 101 - DNA, 21 bp, Unknown
  Oligonucleotide
aagctggtacaaatggaagaa

SEQ ID NO: 102 - DNA, 21 bp, Unknown
  Oligonucleotide
aacaagtccctttgtaaaacc

SEQ ID NO: 103 - DNA, 21 bp, Unknown
  Oligonucleotide
aagaagttcgacaacaagtcc

SEQ ID NO: 104 - DNA, 21 bp, Unknown
  Oligonucleotide
aagttcgacaacaagtccctt

SEQ ID NO: 105 - DNA, 21 bp, Unknown
  Oligonucleotide
aaaaccaaatccaggttctag

SEQ ID NO: 106 - DNA, 21 bp, Unknown
  Oligonucleotide
aaaccaaatccaggttctagc

SEQ ID NO: 107 - DNA, 21 bp, Unknown
  Oligonucleotide
aaccaaatccaggttctagca

SEQ ID NO: 108 - DNA, 21 bp, Unknown
  Oligonucleotide
aaatccaggttctagcacaga

SEQ ID NO: 109 - DNA, 21 bp, Unknown
  Oligonucleotide
aatccaggttctagcacagac

SEQ ID NO: 110 - DNA, 21 bp, Unknown
  Oligonucleotide
aacaacatcctcggttccgaa

SEQ ID NO: 111 - DNA, 21 bp, Unknown
  Oligonucleotide
aacatcctcggttccgaagtg

SEQ ID NO: 112 - DNA, 21 bp, Unknown
  Oligonucleotide
aagtggccttatttgcaggga

SEQ ID NO: 113 - RNA, 21 nt, Unknown
  Oligonucleotide
gcagacagaugcacuauuauu

SEQ ID NO: 114 - RNA, 21 nt, Unknown
  Oligonucleotide
cugcgauuuccaaaucgauuu

SEQ ID NO: 115 - RNA, 21 nt, Unknown
  Oligonucleotide
ggacugguacuauacccacuu